(19) United States
(12) Patent Application Publication
(10) Pub. No.: US 2023/0405015 A1
(43) Pub. Date: Dec. 21, 2023

(54) SUBSTITUTED 3,6-DIHYDRO-2H-1,3,4-OXADIAZIN-2-ONES FOR THE TREATMENT OF SARCOMA

(71) Applicants: Bayer Aktiengesellschaft, Leverkusen (DE); The Broad Institute, Inc., Cambridge, MA (US); Dana-Farber Cancer Institute, Inc., Boston, MA (US)

(72) Inventors: Martin LANGE, Berlin (DE); Stefan KAULFUSS, Berlin (DE); Charlotte Christine KOPITZ, Falkensee (DE); Heidi GREULICH, Cambridge, MA (US); Xiaoyun WU, Cambridge, MA (US); Matthew MEYERSON, Boston, MA (US)

(73) Assignees: Bayer Aktiengesellschaft, Leverkusen (DE); The Broad Institute, Inc., Cambridge, MA (US); Dana-Farber Cancer Institute, Inc., Boston, MA (US)

(21) Appl. No.: 18/241,186

(22) Filed: Aug. 31, 2023

Related U.S. Application Data
(63) Continuation of application No. PCT/EP2022/055211, filed on Mar. 2, 2022.
(60) Provisional application No. 63/156,193, filed on Mar. 3, 2021.

(51) Int. Cl. A61K 31/5395; A61P 35/00

(52) U.S. Cl. CPC A61K 31/5395; A61P 35/00

(57) ABSTRACT

The present invention provides a method of treatment of sarcoma comprising administering to a patient in need thereof a compound of general formula (I),

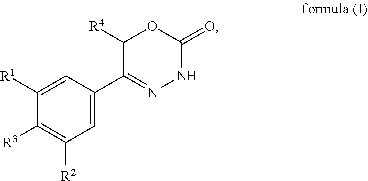

in which R¹, R², R³, and R⁴, are as defined herein, alone or in pharmaceutical compositions or combinations comprising said compounds as a sole agent or in combination with other active ingredients.

### CROSS-REFERENCE TO RELATED APPLICATIONS

This application is a continuation under 35 U.S.C. § 111(a) of PCT International Patent Application No. PCT/EP2022/055211, filed Mar. 2, 2022, designating the United States and published in English, which claims priority to and benefit of U.S. Provisional Patent Application No. 63/156,193, filed Mar. 3, 2021, the entire contents of each of which are incorporated by reference herein.

The present invention provides compounds for the treatment of sarcoma, particularly the use of compounds of general formula (I) as described and defined herein, and the use of their pharmaceutical compositions for the treatment of sarcoma, as a sole agent or in combination with other active ingredients.

### BACKGROUND

Cancer kills over 550,000 people in the United States and over 8 million people world-wide each year. New agents, including small molecules, molecules that impact tissue-specific growth requirements, and immunomodulatory agents, have been shown to benefit a subset of patients whose cancers have unique genomic mutations or other characteristics. Unfortunately, many cancer patients are still left without effective therapeutic options. Sarcomas are a group of cancers for which chemotherapy has been shown to be effective in some cases, leading to increased survival. Nevertheless, chemotherapy has not improved the prognosis of aggressive and metastatic sarcoma patients and there is a high medical need for improved and targeted therapies.

One approach to identify new anti-cancer agents is phenotypic screening to discover novel small molecules displaying strong selectivity between cancer cell lines, followed by predictive chemogenomics to identify the cell features associated with drug response. In the 1990s, Weinstein and colleagues demonstrated that the cytotoxic profile of a compound can be used to identify cellular characteristics, such as gene-expression profiles and DNA copy number, which correlate with drug sensitivity. The ability to identify the features of cancer cell lines that mediate their response to small molecules has strongly increased in recent years with automated high-throughput chemosensitivity testing of large panels of cell lines coupled with comprehensive genomic and phenotypic characterization of the cell lines. Phenotypic observations of small molecule sensitivity can be linked to expression patterns or somatic alterations, as in the case of trastuzumab-sensitive HER2-amplified breast cancer or erlotinib-sensitive EGFR-mutant lung cancer.

Phenotypic screening identified some of the compounds known in the literature to be PDE3 inhibitors to be useful for the treatment of certain cancers. Co-expression of PDE3A and/or PDE3B and Schlafen 12 (SLFN12) polynucleotides or polypeptides are typically required for cells to be sensitive. Those PDE3A/B inhibitors which cause drug sensitivity have been found to stabilize the formation of a complex between PDE3A or PDE3B and SLFN12 and are sometimes even weak PDE3A/PDE3B inhibitors. PDE3A/PDE3B inhibitors which do not cause cell sensitivity typically do not stabilize the PDE3A- or PDE3B-SLFN12 complex.

Sarcomas are cancerous tumors of the connective tissue including blood vessels, bones, cartilage, deep skin tissues, fat, fibrous tissues, lymph vessels, muscles, and nerves. Sarcomas are difficult to diagnose and are not common tumors which start growing in epithelial cells but sarcomas start in mesenchymal cells which build soft tissue and bones. They occur in adults as well as in children. For children even about 15% of all cancer diagnosis are sarcomas. Therefore there seems to be an urgent need to find new therapies.

### SUMMARY

It has now been found that the compounds of formula (I), especially (6S)-5-[4′-fluoro-2-(trifluoromethyl)biphenyl-4-yl]-6-methyl-3,6-dihydro-2H-1,3,4-oxadiazin-2-one, are useful for the treatment of sarcoma.

In accordance with a first aspect, the present invention provides compounds of general formula (I):

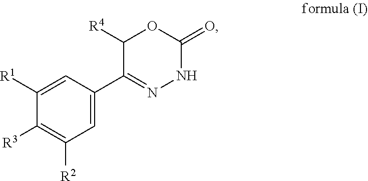

where
R¹ is selected from a hydrogen atom, a halogen atom, a cyano group, a C₁-C₃-alkyl group, a C₁-C₃-haloalkyl group, and a C₁-C₃-haloalkoxy group;
R² is selected from a hydrogen atom and a halogen atom;
R³ is selected from,
   a C₁-C₆-alkyl group which is optionally substituted with one or two substituents and each substituent is independently selected from a hydroxy group, a C₁-C₄-alkoxy group and a 3- to 7-membered heterocycloalkyl group;
   a C₂-C₆-alkenyl group which is optionally substituted with an C₁-C₄-alkoxy group;
   a C₃-C₉-cycloalkyl group, which is optionally substituted with a hydroxy group;
   a C₅-C₉-cycloalkenyl group, which is optionally substituted with a hydroxy group;
   a 3- to 9-membered heterocycloalkyl group, comprising one, two or three heteroatoms which are independently selected from —O—, —S—, —S(O)—, S(O)₂, and —NR⁹—,
      and said heterocycloalkyl group optionally further comprising a bridging group selected from —O—, —NR⁹—, —CH₂—, —CH₂—CH₂—, —O—CH₂—, —CH₂—O—, —NR⁹—CH₂—, and —CH₂—NR⁹—;
      and said heterocycloalkyl group is optionally substituted with one, two or three substituents and each substituent is independently selected from
      a halogen atom;
      a oxo (═O) group;
      a cyano group;
      a hydroxy group;
      a C₁-C₃-alkyl group which is optionally further substituted with a hydroxy group;
      a C₁-C₃-haloalkyl group;
      a C₁-C₃-alkoxy group;
      a C₁-C₃-haloalkoxy group;
      a C(O)NR⁵R⁶ group
      and a NR⁵R⁶ group;
   a 5- to 9-membered heterocycloalkyl group which is partially unsaturated and optionally substituted with one, two or three substituents and each substituent is independently selected from an oxo group (═O), a C₁-C₃-alkyl group, a —C(O)R⁵R⁶ group and a halogen atom;
   an aryl group which is optionally substituted with one, two, three or four substituents and each substituent is independently selected from a halogen atom, a hydroxy group, a cyano group, a C₁-C₃-alkyl group, a C₁-C₃-haloalkyl group, a C₁-C₃-alkoxy group, a C₁-C₃-haloalkoxy group, and a NR⁵R⁶ group;
   a mono- or bicyclic heteroaryl group which is optionally substituted with one, two or three substituents and each substituent is independently selected from a halogen atom, a C₁-C₃-alkyl group, a cyano group, a C₁-C₃-haloalkyl group, a C₁-C₃-hydroxalkyl group, a C₁-C₃-alkoxy group, a hydroxy group, and a NR⁵R⁶ group, with the proviso that said monocyclic heteroaryl group is not 4-pyridyl;
   and a NR⁷R⁸ group;
R⁴ is selected from a hydrogen atom, and a C₁-C₃-alkyl group;
R⁵/R⁶ are independently selected from a hydrogen atom, a C₁-C₆-alkyl group, a —C₁-C₅-alkylene-O—C₁-C₅-alkyl group, a —C₁-C₅-alkylene-S—C₁-C₅-alkyl group, C₃-C₆-cycloalkyl group, and a C₃-C₅-heterocycloalkyl group;
R⁷/R⁸ are independently selected from a hydrogen atom, with the proviso that R⁷=R⁸=hydrogen is excluded,
   a C₁-C₆-alkyl group,
   which is optionally substituted with one, two, three or four substituents and said substituent is independently selected from
      a halogen atom,
      a cyano group,
      a hydroxy group,
      a C(O)NR⁵R⁶ group,
      a NR⁵R⁶ group,
      a C₁-C₃-alkoxy group,
      a C₃-C₇-cycloalkyl group which is optionally substituted with one or two substituents and said substituents are independently selected from a C₁-C₃-alkyl group, a oxo (═O) group, a hydroxy group, and a C₁-C₃-hydroxyalkyl group;
      a 3- to 7-membered heterocycloalkyl group which itself is optionally substituted with, a C₁-C₃-alkyl group or an oxo (═O) group;
      a heteroaryl group, which itself is optionally substituted with a C₁-C₃-alkyl group;
   a —C₁-C₅-alkylene-O—C₁-C₅-alkyl group;
   a —C₁-C₅-alkylene-S—C₁-C₅-alkyl group;
   a —C₁-C₅-alkylene-NR⁵—C₁-C₅-alkyl group;
   a C₃-C₆-cycloalkyl group which is optionally substituted with a hydroxy group; and a 3- to 6-membered heterocycloalkyl group, which is optionally substituted with one or two substituents, said substituent independently selected from C₁-C₃-alkyl group and a hydroxy group;
R⁹ is a hydrogen atom or a C₁-C₃-alkyl group or a bond;
   or a stereoisomer, a tautomer, a hydrate, a solvate, or a salt thereof, or a mixture of same for use in the treatment of sarcoma. The compounds of general formula (I) may be used, for example, for the treatment, prophylaxis, or control of sarcomas in a subject in need thereof as described herein.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Structures drawn include all permissible rotations about bonds.

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom.

Commonly, it is possible for the number of optional substituents, when present, to be 1, 2, 3, in particular 1, or 2.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

Should a composite substituent be composed of more than one parts, e.g. (C₁-C₄-alkyl)-O—(C₁-C₄-alkyl)-, a hyphen at the beginning or at the end of such a composite substituent indicates the point of attachment of said composite substituent to the rest of the molecule. Should the composite substituent be substituted said substituent may be bound at any suitable carbon atom of the composite substituent.

Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “alkylene” derives from the term “alkyl” as being a bivalent constituent named by addition of “ene” to the term “alkyl” e.g. “methyl” becomes “methylene” meaning a “—CH₂—” constituent whereby the open bonds of branched constituents are located at the respective ends of the longest chain.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl, more particularly trifluoromethyl or difluoromethyl.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₂-C₆-alkenyl” means a linear or branched, monovalent hydrocarbon group, which contains one or two double bonds, and which has 2, 3, 4, 5 or 6 carbon atoms, particularly 2 or 4 carbon atoms (“C₂-C₄-alkenyl”), it being understood that in the case in which said alkenyl group contains more than one double bond, then it is possible for said double bonds to be isolated from, or conjugated with, each other. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group. Particularly, said group is vinyl or allyl, propenyl-, isopropenyl-, butenyl-, or isobutenyl group.

The term “C₂-C₆-alkynyl” means a linear or branched, monovalent hydrocarbon group which contains one triple bond, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methyl-pent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group. Particularly, said alkynyl group is ethynyl, prop-1-ynyl or prop-2-ynyl.

The term “C₃-C₉-cycloalkyl” means a saturated, monovalent, mono- or bicyclic hydrocarbon ring which contains 3, 4, 5, 6, 7 or 8 carbon atoms. Said C₃-C₈-cycloalkyl group is for example, a monocyclic hydrocarbon ring, e.g. a cyclopropyl, cyclobutyl, cyclopentyl, cyclohexyl, cycloheptyl or cyclooctyl group, it also includes fused-, bridged- and spiro-cycloalkyl ring systems as e.g. a bicyclic hydrocarbon ring, e.g. a bicyclo[4.2.0]octyl, bicyclo[2.2.1]heptyl or octahydropentalenyl as well as spirocycoalkyl systems as defined below.

The term “spirocycloalkyl” means a saturated, monovalent bicyclic hydrocarbon group in which the two rings share one common ring carbon atom, and wherein said bicyclic hydrocarbon group contains 5, 6, 7, 8, or 9 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl, spiro[2.4]heptyl, spiro[2.5]octyl, spiro[2.6]nonyl, spiro[3.3]heptyl, spiro[3.4]octyl, spiro[3.5]nonyl, spiro[3.6]decyl, spiro[4.4]nonyl, spiro[4.5]decyl, spiro[4.6]undecyl or spiro[5.5]undecyl.

The term “C₅-C₆-cycloalkenyl” means a resulting a cyclopentenyl group, a cyclohexenyl group, a cyclopentadienyl group a cyclohexadienyl group

The term “C₄-C₉-cycloalkenyl” means a monovalent, mono- or bicyclic hydrocarbon ring which contains 4, 5, 6, 7, 8 or 9 carbon atoms and one double bond. Particularly, said ring contains 4, 5 or 6 carbon atoms (“C₄-C₆-cycloalkenyl”). Said C₄-C₈-cycloalkenyl group is for example, a monocyclic hydrocarbon ring, e.g. a cyclobutenyl, cyclopentenyl, cyclohexenyl, cycloheptenyl or cyclooctenyl group, or a bridged ring system also a bicyclic hydrocarbon ring, e.g. a bicyclo[2.2.1]hept-2-enyl or bicyclo[2.2.2]oct-2-enyl, bicyclo[3.1.0]hex-2-enyl.

The terms “3- to 9-membered heterocycloalkyl” and “3- to 6-membered heterocycloalkyl” mean a saturated heterocycle with 3, 4, 5, 6, 7, 8 or 9 ring atoms respectively, 3, 4, 5 or 6 ring atoms in total, which contains one or two identical or different ring heteroatoms selected from the series N, O, and S, said heterocycloalkyl group being attached to the rest of the molecule via any one of the carbon atoms or heteroatoms. These also include bicyclic ring systems which are either fused- or bridged- or spiro-systems as defined below. It also includes compounds of formula (I) having potentially a NR⁷R⁸ group where the N-atom belongs to a ring which is being formed by connection of R⁷ and R⁸ forming a non-aromatic ring including the N-atom to which they are connected. The term “heterocycloalkane”, as used herein, refers to a compound consisting of a heterocycloalkyl group as defined herein, and a hydrogen atom to which said heterocycloalkyl group is bonded with its one valency.

Said heterocycloalkyl group, without being limited thereto, can be a 3- or 4-membered ring, such as azacyclopropyl, oxacyclopropyl, azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, or a fused system like azabicyclo[3.1.0]hexan-3-yl for example.

Particularly, “4- to 6-membered heterocycloalkyl” means a 4- to 6-membered heterocycloalkyl as defined supra containing one ring nitrogen atom or an oxygen atom or a sulfur atom and if it contains a nitrogen atom it may optionally contains one further ring heteroatom from the series: N, O, S. More particularly, “5- or 6-membered heterocycloalkyl” means a monocyclic, saturated heterocycle with 5 or 6 ring atoms in total, containing one ring nitrogen atom and optionally one further ring heteroatom from the series: N, O. Said heterocycloalkyl group is being attached to the rest of the molecule via any carbon atom or where applicable via any nitrogen atom. Both of them may include bicyclic ring systems as mentioned above. The “5- or 6-membered heterocycloalkyl” group can be particularly substituted with one or two fluorine atoms or a methyl group.

The term a “partially unsaturated 3- to 9-membered heterocycloalkyl” means a monocyclic, unsaturated, non-aromatic heterocycle with 5, 6, 7, 8 or 9 ring atoms in total, which contains one or two double bonds and one or two identical or different ring heteroatoms from the series: N, O, S; it being possible for said partially unsaturated heterocycloalkyl group to be attached to the rest of the molecule via any one of the carbon atoms or, if present, a nitrogen atom. The term “partially unsaturated heterocycloalkane”, as used herein, refers to a compound consisting of a partially unsaturated heterocycloalkyl group as defined herein, and a hydrogen atom to which said partially unsaturated heterocycloalkyl group is bonded with its one valency.

Said partially unsaturated heterocycloalkyl group is, for example, 4H-pyranyl, 2H-pyranyl, 3,6-dihydro-2H-pyran-4-yl, 5,6-dihydro-2H-pyran-3-yl, tetrahydropyridinyl, e.g. 1,2,3,6-tetrahydropyridin-4-yl, dihydropyridinyl, e.g. 1,6-dihydropyridinyl, 6-oxo-1,6-dihydropyridin-3-yl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl or 4H-[1,4]thiazinyl.

The term “fused heterocycloalkyl” means a bicyclic, saturated heterocycle with 6, 7, 8, or 9 ring atoms in total, or respectively 5, 6 or 7 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl group is, for example, 3-azabicyclo[3.1.0]hexan-3-yl, 3-azabicyclo[3.2.0]heptan-3-yl, azabicyclo[3.3.0]octyl, azabicyclo[4.3.0]nonyl, diazabicyclo[4.3.0]nonyl, oxazabicyclo[4.3.0]nonyl, or thiazabicyclo[4.3.0]nonyl.

The term “bridged heterocycloalkyl” means a bicyclic, saturated heterocycle with 7, 8 or 9 ring atoms in total, or respectively 7 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms or, if present, a nitrogen atom. Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo[3.2.1]octyl, oxazabicyclo[3.2.1]octyl, thiazabicyclo[3.2.1]octyl, azabicyclo[3.3.1]nonyl, diazabicyclo[3.3.1]nonyl, oxazabicyclo[3.3.1]nonyl, thiazabicyclo[3.3.1]-nonyl, azabicyclo[4.2.1]nonyl, diazabicyclo[4.2.1]nonyl, oxazabicyclo[4.2.1]nonyl, thiazabicyclo[4.2.1]nonyl.

The term “heterospirocycloalkyl” means a bicyclic, saturated heterocycle with 6, 7, 8, or 9 ring atoms in total, in which the two rings share one common ring carbon atom, which “heterospirocycloalkyl” contains one or two identical or different ring heteroatoms from the series: N, O, S; it being possible for said heterospirocycloalkyl group to be attached to the rest of the molecule via any one of the carbon atoms, except the spiro carbon atom, or, if present, a nitrogen atom. Said heterospirocycloalkyl group is, for example, azaspiro[2.3]hexyl, azaspiro[3.3]heptyl, oxaazaspiro[3.3]heptyl, thiaazaspiro[3.3]heptyl, 2lambda<sup>6</sup>-thia-6-azaspiro[3.3]heptane-2,2-dione, oxaspiro[3.3]heptyl, oxazaspiro[5.3]nonyl, oxazaspiro[4.3]octyl, diazaspiro[3.3]heptyl, thiazaspiro[3.3]heptyl, thiazaspiro[4.3]octyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “aryl” refers to an aromatic monocyclic, bicyclic (2 fused rings), tricyclic (3 fused rings), or polycyclic (two or more fused rings) hydrocarbon ring system having 6 to 20 (e.g. 6 to 10 ring carbon atoms). Nonlimiting examples of aryl groups include phenyl, or naphthyl (e.g., 1-napthyl, 2-napthyl, etc.). Particularly the aryl group is a phenyl group which is optionally substituted with one or two substituents independently selected from fluorine, difluoromethyl and trifluoromethyl.

The term “heteroaryl” means a monovalent, monocyclic or bicyclic aromatic ring having 5, 6, 8, 9 or 10, ring atoms (a “5- to 10-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or a heteroatom to the rest of the molecule. The term “heteroarene”, as used herein, refers to a compound consisting of a heteroaryl group as defined herein, and a hydrogen atom to which said heteroaryl group is bonded with its one valency.

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

Particularly the heteroaryl group is a 2H-pyrrol-1-yl group, a 1H-pyrrazol-4-yl group, a 1H-pyrrazol-5-yl group, which is optionally substituted with one or two methyl groups, a 1,2-thiazol-4-yl group, a 1,3-thiazol-5-yl group, a pyridin-3-yl, a pyridin-4-yl and a pyridin 5-yl group each group being optionally substituted with one or two substituents and each substituent is independently selected from a halogen atom, a methyl group, a trifluoromethyl group, a methoxy group and a NH₂ group, a 1H-indol-6-yl group, a 1H-indazol-6-yl group, and a 1H-benzimidazol-6-yl group, each group being optionally substituted with one or two substituents and each substituent is independently selected from a halogen atom, a methyl group, a trifluoromethyl group, a methoxy group and a NH₂ group.

Particularly, the heteroaryl group is a:
2H-pyrrol-1-yl group, which is optionally substituted with one or two substituents and each substituent is independently selected from a hydrogen atom, a cyano group and a methyl group,
   a 1H-pyrrazol-4-yl group, which is optionally substituted with one or two methyl groups,
   a 1H-pyrrazol-5-yl group, which is optionally substituted with one or two methyl groups,
   a 1,2-thiazol-4-yl group which is optionally substituted with one or two methyl groups,
   a 1,3-thiazol-5-yl group which is optionally substituted with one or two methyl groups,
   a pyridin-3-yl, and a pyridin 5-yl group each group being optionally substituted with one or two substituents and each substituent is independently selected from a halogen atom, a methyl group, a trifluoromethyl group, a methoxy group and a NH₂ group,
   a 1H-indol-6-yl group, a 1H-indazol-6-yl group, and a 1H-benzimidazol-6-yl group.

More particularly the heteroaryl group is a pyridinyl group which is optionally substituted with an amino group, or a pyrazolyl group which is optionally substituted with a difluoromethyl group or a trifluoromethyl group.

Even more particularly the heteroaryl group is
a 1H-pyrazol-4-yl or a 1H-pyrazol-1-yl group which are optionally substituted with a difluoromethyl group or a trifluoromethyl group

The term “C₁-C₆”, as used in the present text, e.g. in the context of the definition of “C₁-C₆-alkyl”, “C₁-C₆-haloalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

Ring heteroatoms may be substituted to the extent permitted by valency (and, where appropriate, aromaticity), for example, to include —S(O)—, —S(O)₂—, and —N(R)— as heteroatoms in the ring system (where, for example, R may be a hydrogen atom or a C₁-C₃-alkyl group).

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄-C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Controlling” a disease such as sarcoma may refer to any reduction in the rate of the progression of the disease.

“Detect” refers to identifying the presence, absence or amount of the analyte to be detected. In particular embodiments, the analyte is a PDE3A or SLFN12 polypeptide.

By “disease” is meant any condition or disease that damages or interferes with the normal function of a cell, tissue, or organ. Examples of diseases include sarcomas (e.g., malignant fibrous histiocytoma, lymphosarcoma, osteosarcoma, rhabdomyosarcoma, soft tissue sarcoma, and synovial sarcoma).

By “effective amount” is meant the amount of a compound described herein that inhibits the growth or proliferation of a sarcoma relative to the growth or proliferation of the sarcoma in an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butylphenyl)sulfonyl]oxy and [(4-methoxyphenyl)sulfonyl]oxy.

Unless specifically stated or obvious from context, as used herein, the term “or” is understood to be inclusive. Unless specifically stated or obvious from context, as used herein, the terms “a”, “an”, and “the” are understood to be singular or plural.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The term “prodrugs” or “prodrug” designates compounds which themselves can be biologically active or inactive, but are converted (for example metabolically or hydrolytically) into compounds of formula (I) during their residence time in the body. Derivatives of the compound 6 and the salts thereof which are converted into compound 6 or a salt thereof in a biological system (bioprecursors or pro-drugs) are covered by the invention. Said biological system may be, for example, a mammalian organism, particularly a human subject. The bioprecursor is, for example, converted into a compound of formula (I) or a salt thereof by metabolic processes.

The term “pharmaceutically acceptable salt(s)” of the compounds of formula (I) include those derived from pharmaceutically acceptable inorganic and organic acids and bases. For example, see S. M. Berge, et al. “Pharmaceutical Salts,” J. Pharm. Sci. 1977, 66, 1-19.

As used herein, the term “pharmaceutically acceptable salt” refers to a salt formed by the addition of a pharmaceutically acceptable acid or base to a compound disclosed herein.

As used herein, the phrase “pharmaceutically acceptable” refers to a substance that is acceptable for use in pharmaceutical applications from a toxicological perspective and does not adversely interact with the active ingredient.

As used herein, “sarcoma or sarcomas” include, but are not limited to, sarcoma of the soft tissue and bone, more particularly osteosarcoma, malignant fibrous histiocytoma, soft tissue sarcoma, synovial sarcoma, lymphosarcoma, and rhabdomyosarcoma. In some embodiments, a sarcoma is not a soft tissue sarcoma, osteosarcoma, malignant fibrous histiocytoma, lymphosarcoma, or rhabdomyosarcoma, in some embodiments sarcoma does not include rabdomyosarcoma or lymphosarcoma.

By “subject” is meant a mammal, including, but not limited to, a human or non-human mammal, such as a bovine, equine, canine, ovine, rodent, or feline. A subject in need thereof is typically a subject for whom it is desirable to treat a disease, disorder, or condition as described herein (e.g., sarcoma). For example, a subject in need thereof may seek or be in need of treatment, require treatment, be receiving treatment, may be receiving treatment in the future, or a human or animal that is under care by a trained professional for a particular disease, disorder, or condition.

Unless specifically stated or obvious from context, as used herein, if a range is provided, the upper and lower limit are always meant to be included. Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reference” is meant a standard or control condition which can be a healthy cell or an average expression in a representative panel of tumor cells or tumor cell lines. For example, the growth or proliferation of a sarcoma treated with a compound described herein is compared to the growth or proliferation of an untreated sarcoma, which acts as a reference.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Preferred isomers are those which produce the more desirable biological activity. These separated, pure or partially purified isomers or racemic mixtures of the compounds for use in this invention are also included within the scope of the present invention. The purification and the separation of such materials can be accomplished by standard techniques known in the art.

Thus as one embodiment of the invention for the use of compounds of formula (I) for the treatment of sarcoma, the configuration of the alkyl group in R⁴ specifically for R⁴=C₁-C₃-alkyl, more particularly R⁴=methyl is S-configuration as indicated in formula (Ia)

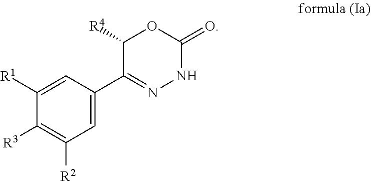

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

Further, it is possible for the compounds for use in the present invention may exist as tautomers. For example, any compound of the present invention which contains an imidazopyridine moiety as a heteroaryl group for example can exist as a 1H tautomer, or a 3H tautomer, or even a mixture in any amount of the two tautomers, namely

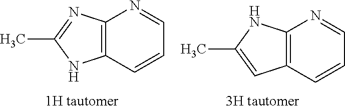

For the use of the compounds of Formula (I) all possible tautomers as single tautomers, or as any mixture of said tautomers, in any ratio can be used.

Useful forms of the compounds for use in the present invention for the treatment of sarcoma may be such as metabolites, hydrates, solvates, prodrugs, salts, in particular pharmaceutically acceptable salts, and/or co-precipitates.

They can also exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta-etc. solvates or hydrates, respectively, are possible. The present invention includes all such hydrates or solvates.

Furthermore, the compounds for use in the present invention includes all possible crystalline forms, or polymorphs, of the compounds of formula (I), either as single polymorph, or as a mixture of more than one polymorph, in any ratio.

### BRIEF DESCRIPTION OF THE DRAWINGS

FIG. 1:

In vivo anti-tumor efficacy of (6S)-5-[4′-fluoro-2-(trifluoromethyl)biphenyl-4-yl]-6-methyl-3,6-dihydro-2H-1,3,4-oxadiazin-2-one (“the Compound”) in a sarcoma patient-derived xenograft model. Tumor growth of the sarcoma patient derived xenograft model SA3831 subcutaneously on immune compromised mice treated with the Compound or vehicle. The Compound demonstrated significant anti-tumor efficacy with a T (Treated)/C (vehicle control) ratio of 0.01. The T/C ratio is based on the final tumor sizes and the statistical assessment has been done by an unpaired t-test (ns=P>0.05, *=P≤0.05, **=P≤0.01, ***=P≤0.001).

FIG. 2:

In vivo anti-tumor efficacy of (6S)-5-[4′-fluoro-2-(trifluoromethyl)biphenyl-4-yl]-6-methyl-3,6-dihydro-2H-1,3,4-oxadiazin-2-one in a sarcoma patient-derived xenograft model. Tumor growth of the sarcoma patient derived xenograft model SA4058 subcutaneously on immune compromised mice treated with the Compound or vehicle. The Compound demonstrated significant anti-tumor efficacy with a T (Treated)/C (vehicle control) ratio of 0.12. The T/C ratio is based on the final tumor sizes and the statistical assessment has been done by an unpaired t-test (ns=P>0.05, *=P≤0.05, **=P≤0.01, ***=P≤0.001).

FIG. 3:

In vivo anti-tumor efficacy of (6S)-5-[4′-fluoro-2-(trifluoromethyl)biphenyl-4-yl]-6-methyl-3,6-dihydro-2H-1,3,4-oxadiazin-2-one in a sarcoma patient-derived xenograft model. Tumor growth of the sarcoma patient derived xenograft model SA16044 subcutaneously on immune compromised mice treated with the Compound or vehicle. The Compound demonstrated significant anti-tumor efficacy with a T (Treated)/C (vehicle control) ratio of 0.35. T/C ratio is based on the final tumor sizes and the statistical assessment has been done by an unpaired t-test (ns=P>0.05, *=P≤0.05, **=P≤0.01, ***=P≤0.001).

FIG. 4:

In vivo anti-tumor efficacy of (6S)-5-[4′-fluoro-2-(trifluoromethyl)biphenyl-4-yl]-6-methyl-3,6-dihydro-2H-1,3,4-oxadiazin-2-one in a sarcoma patient-derived xenograft model. Tumor growth of the sarcoma patient derived xenograft model SA10199 subcutaneously on immune compromised mice treated with (6S)-5-[4′-fluoro-2-(trifluoromethyl)biphenyl-4-yl]-6-methyl-3,6-dihydro-2H-1,3,4-oxadiazin-2-one or vehicle. (6S)-5-[4′-fluoro-2-(trifluoromethyl)biphenyl-4-yl]-6-methyl-3,6-dihydro-2H-1,3,4-oxadiazin-2-one demonstrated significant anti-tumor efficacy with a T (Treated)/C (vehicle control) ratio of 0.31. The T/C ratio is based on the final tumor sizes and the statistical assessment has been done by an unpaired t-test (ns=P>0.05, *=P≤0.05, **=P≤0.01,***=P≤0.001).

FIG. 5:

In vivo anti-tumor efficacy of (6S)-5-[4′-fluoro-2-(trifluoromethyl)biphenyl-4-yl]-6-methyl-3,6-dihydro-2H-1,3,4-oxadiazin-2-one in a sarcoma patient-derived xenograft model. Tumor growth of the sarcoma patient derived xenograft model SA10245 subcutaneously on immune compromised mice treated with (6S)-5-[4′-fluoro-2-(trifluoromethyl)biphenyl-4-yl]-6-methyl-3,6-dihydro-2H-1,3,4-oxadiazin-2-one or vehicle. (6S)-5-[4′-fluoro-2-(trifluoromethyl)biphenyl-4-yl]-6-methyl-3,6-dihydro-2H-1,3,4-oxadiazin-2-one demonstrated non-significant signs of anti-tumor efficacy with a T (Treated)/C (vehicle control) ratio of 0.73. The T/C ratio is based on the final tumor sizes and the statistical assessment has been done by an unpaired t-test (ns=P>0.05, *=P≤0.05, **=P≤0.01, ***=P≤0.001).

### DETAILED DESCRIPTION

In accordance with a first aspect, the present invention includes the method of treatment of sarcoma comprising administering to a patient in need thereof a compound of general formula (I):

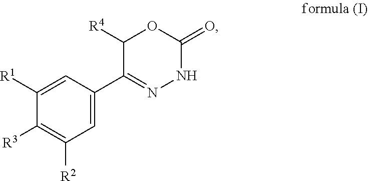

where
R¹ is selected from a hydrogen atom, a halogen atom, a cyano group, a C₁-C₃-alkyl group, a C₁-C₃-haloalkyl group, and a C₁-C₃-haloalkoxy group;
R² is selected from a hydrogen atom and a halogen atom;
R³ is selected from,
   a C₁-C₆-alkyl group which is optionally substituted with one or two substituents and each substituent is independently selected from a hydroxy group, a C₁-C₄-alkoxy group and a 3- to 7-membered heterocycloalkyl group;
   a C₂-C₆-alkenyl group which is optionally substituted with an C₁-C₄-alkoxy group;
   a C₃-C₉-cycloalkyl group, which is optionally substituted with a hydroxy group;
   a C₅-C₉-cycloalkenyl group, which is optionally substituted with a hydroxy group;
   a 3- to 9-membered heterocycloalkyl group, comprising one, two or three heteroatoms which are independently selected from —O—, —S—, —S(O)—, S(O)₂, and —NR⁹—,
      and said heterocycloalkyl group optionally further comprising a bridging group selected from —O—, —NR⁹—, —CH₂—, —CH₂—CH₂—, —O—CH₂—, —CH₂—O—, —NR⁹—CH₂—, and —CH₂—NR⁹—;
      and said heterocycloalkyl group is optionally substituted with one, two or three substituents and each substituent is independently selected from
      a halogen atom;
      a oxo (═O) group;
      a cyano group;
      a hydroxy group;
      a C₁-C₃-alkyl group which is optionally further substituted with a hydroxy group;
      a C₁-C₃-haloalkyl group;
      a C₁-C₃-alkoxy group;
      a C₁-C₃-haloalkoxy group;
      a C(O)NR⁵R⁶ group
      and a NR⁵R⁶ group;
   a 5- to 9-membered heterocycloalkyl group which is partially unsaturated and optionally substituted with one, two or three substituents and each substituent is independently selected from an oxo group (═O), a C₁-C₃-alkyl group, a —C(O)R⁵R⁶ group and a halogen atom;
   an aryl group which is optionally substituted with one, two, three or four substituents and each substituent is independently selected from a halogen atom, a hydroxy group, a cyano group, a C₁-C₃-alkyl group, a C₁-C₃-haloalkyl group, a C₁-C₃-alkoxy group, a C₁-C₃-haloalkoxy group, and a NR⁵R⁶ group;
   a mono- or bicyclic heteroaryl group which is optionally substituted with one, two or three substituents and each substituent is independently selected from a halogen atom, a C₁-C₃-alkyl group, a cyano group, a C₁-C₃-haloalkyl group, a C₁-C₃-hydroxalkyl group, a C₁-C₃-alkoxy group, a hydroxy group, and a NR⁵R⁶ group, with the proviso that said monocyclic heteroaryl group is not 4-pyridyl;
   and a NR⁷R⁸ group;
R⁴ is selected from a hydrogen atom, and a C₁-C₃-alkyl group;
R⁵/R⁶ are independently selected from a hydrogen atom, a C₁-C₆-alkyl group, a —C₁-C₅-alkylene-O—C₁-C₅-alkyl group, a —C₁-C₅-alkylene-S—C₁-C₅-alkyl group, C₃-C₆-cycloalkyl group, and a C₃-C₅-heterocycloalkyl group;
R⁷/R⁸ are independently selected from a hydrogen atom, with the proviso that R⁷=R⁸=hydrogen is excluded,
   a C₁-C₆-alkyl group,
   which is optionally substituted with one, two, three or four substituents and said substituent is independently selected from
      a halogen atom,
      a cyano group,
      a hydroxy group,
      a C(O)NR⁵R⁶ group,
      a NR⁵R⁶ group,
      a C₁-C₃-alkoxy group,
      a C₃-C₇-cycloalkyl group which is optionally substituted with one or two substituents and said substituents are independently selected from a C₁-C₃-alkyl group, a oxo (═O) group, a hydroxy group, and a C₁-C₃-hydroxyalkyl group;
      a 3- to 7-membered heterocycloalkyl group which itself is optionally substituted with, a C₁-C₃-alkyl group or an oxo (═O) group;
      a heteroaryl group, which itself is optionally substituted with a C₁-C₃-alkyl group;
   a —C₁-C₅-alkylene-O—C₁-C₅-alkyl group;
   a —C₁-C₅-alkylene-S—C₁-C₅-alkyl group;
   a —C₁-C₅-alkylene-NR⁵—C₁-C₅-alkyl group;
   a C₃-C₆-cycloalkyl group which is optionally substituted with a hydroxy group; and
   a 3- to 6-membered heterocycloalkyl group, which is optionally substituted with one or two substituents, said substituent independently selected from C₁-C₃-alkyl group and a hydroxy group;
R⁹ is a hydrogen atom or a C₁-C₃-alkyl group or a bond;
   or a stereoisomer, a tautomer, a hydrate, a solvate, or a salt thereof, or a mixture of same or the compounds mentioned above for use in the treatment of sarcoma.

In accordance with a second embodiment of the first aspect, the present invention includes the method of treatment of sarcoma comprising administering to a patient in need thereof a compound of general formula (I), supra, in which:
R¹ is selected from a hydrogen atom, a halogen atom, a cyano group, a C₁-C₃-alkyl group, a C₁-C₃-haloalkyl group, and a C₁-C₃-haloalkoxy group;
R² is selected from a hydrogen atom and a halogen atom;
R³ is selected from
   a C₁-C₆-alkyl group which is optionally substituted with a substituent which is selected from a hydroxy group, a C₁-C₄-alkoxy group, and a 3- to 7-membered heterocycloalkyl group;
   a C₂-C₆-alkenyl group which is optionally substituted with an C₁-C₄-alkoxy group;
   a C₃-C₇-cycloalkyl group, which is optionally substituted with a hydroxy group;
   a C₅-C₇-cycloalkenyl group, which is optionally substituted with a hydroxy group;
   a 3- to 7-membered-heterocycloalkyl group, comprising one, or two heteroatoms which are independently selected from —O—, S(O)₂, and —NR⁹—,
      and said heterocycloalkyl group is optionally substituted with one, or two substituents
      and each substituent is independently selected from
      a halogen atom;
      a cyano group;
      a hydroxy group;
      a C₁-C₃-alkyl group which is optionally further substituted with a hydroxy group;
      a C₁-C₃-alkoxy group;
      a C(O)NR⁵R⁶ group and
      a NR⁵R⁶ group;
   a 5- to 7-membered-heterocycloalkyl group, comprising a heteroatom which is selected from —O—, —S— and —NR⁹—, which is partially unsaturated and optionally substituted with a substituent which is selected from a C₁-C₃-alkyl group and a halogen atom;
   an aryl group which is optionally substituted with one, two, or three substituents and each substituent is independently selected from a halogen atom, a hydroxy group, a cyano group, a C₁-C₃-alkyl group, a C₁-C₃-haloalkyl group, a C₁-C₃-alkoxy group, and a NR⁵R⁶ group;
   a mono- or bicyclic heteroaryl group which is optionally substituted with a substituent which is selected from a halogen atom, a C₁-C₃-alkyl group, a cyano group, a C₁-C₃-haloalkyl group, a C₁-C₃-alkoxy group, and a NR⁵R⁶ group, with the proviso that said monocyclic heteroaryl group is not a pyridin-4-yl group;
   and a NR⁷R⁸ group;
R⁴ is selected from a hydrogen atom, and a C₁-C₃-alkyl group;
R⁵/R⁶ are independently selected from a hydrogen atom and a C₁-C₆-alkyl group;
R⁷/R⁸ are independently selected from
   a hydrogen atom, with the proviso that R⁷=R⁸=hydrogen is excluded,
   a C₁-C₆-alkyl group, which is optionally substituted with one, two, three or four substituents and said substituent is independently selected from
      a halogen atom;
      a cyano group;
      a hydroxy group;
      a C(O)NR⁵R⁶ group;
      a NR⁵R⁶ group;
      a C₁-C₃-alkoxy group;
      a C₃-C₇-cycloalkyl group which is optionally further substituted with one or two substituents and said substituents are independently selected from a C₁-C₃-alkyl group, a oxo (═O) group, a hydroxy group, and a C₁-C₃-hydroxyalkyl group;
      a 3- to 7-membered heterocycloalkyl group, comprising one, two or three heteroatoms which are independently selected from —O— and —NR⁹—, which is optionally further substituted with a C₁-C₃-alkyl group;
      a heteroaryl group, which is optionally further substituted with a C₁-C₃-alkyl group;
   a C₃-C₇-cycloalkyl group which is optionally substituted with a hydroxy group, or a C₁-C₃-alkyl group and
   a 3- to 6-membered heterocycloalkyl group, which is optionally substituted with one or two substituents, said substituent independently selected from C₁-C₃-alkyl group and a hydroxy group;
R⁹ is a hydrogen atom or a C₁-C₃-alkyl group or a bond;
   or a stereoisomer, a tautomer, a hydrate, a solvate, or a salt thereof, or a mixture of same or the compounds mentioned above for use in the treatment of sarcoma.

In accordance with a third embodiment of the first aspect, the present invention includes the method of treatment of sarcoma comprising administering to a patient in need thereof a compound of general formula (I), supra, in which:
R¹ is selected from a hydrogen atom, a halogen atom, a C₁-C₃-alkyl group, a C₁-C₃-haloalkyl group, and a C₁-C₃-haloalkoxy group;
R² is selected from a hydrogen atom and a halogen atom;
R³ is selected from,
   a C₁-C₆-alkyl group which is optionally substituted with a substituent which is selected from a hydroxy group, a C₁-C₄-alkoxy group, and a 3- to 7-membered heterocycloalkyl group;
   a C₂-C₆-alkenyl group which is optionally substituted with an C₁-C₄-alkoxy group;
   a C₃-C₇-cycloalkyl group, which is optionally substituted with a hydroxy group;
   a C₅-C₇-cycloalkenyl group;
   a 3- to 7-membered heterocycloalkyl group, comprising one, or two heteroatoms which are independently selected from —O—, and —NR⁹—,
      and said heterocycloalkyl group is optionally further substituted with one, or two substituents and each substituent is independently selected from
      a halogen atom;
      a cyano group;
      a hydroxy group;
      a C₁-C₃-alkyl group which is optionally further substituted with a hydroxy group;
      a C₁-C₃-alkoxy group and
      a C(O)NR⁵R⁶ group;
   a 5- to 7-membered-heterocycloalkyl group, comprising a heteroatom which is selected from —O—, and —NR⁹—, which is partially unsaturated and optionally substituted with a substituent which is selected from a C₁-C₃-alkyl group and a halogen atom,
   an aryl group which is optionally substituted with one, two, or three substituents and each substituent is independently selected from a halogen atom, a hydroxy group, a cyano group, a C₁-C₃-alkyl group, a C₁-C₃-haloalkyl group, and a NR⁵R⁶ group;
   a mono- or bicyclic heteroaryl group which is optionally substituted with a substituent which is selected from a halogen atom, a C₁-C₃-alkyl groupa C₁-C₃-haloalkyl group, a C₁-C₃-alkoxy group, and a NR⁵R⁶ group, with the proviso that said monocyclic heteroaryl group is not a pyridin-4-yl group;
   and a NR⁷R⁸ group;
R⁴ is selected from a hydrogen atom, and a C₁-C₃-alkyl group;
R⁵/R⁶ are independently selected from a hydrogen atom and a C₁-C₆-alkyl group;
R⁷/R⁸ are independently selected from
   a hydrogen atom, with the proviso that R⁷=R⁸=hydrogen is excluded,
   a C₁-C₆-alkyl group, which is optionally substituted with one, two, three or four substituents and said substituent is independently selected from
      a halogen atom;
      a cyano group;
      a hydroxy group;
      a NR⁵R⁶ group;
      a C₁-C₃-alkoxy group;
      a C₃-C₇-cycloalkyl group which is optionally further substituted with one or two substituents and said substituents are independently selected from a C₁-C₃-alkyl group, a oxo (═O) group, a hydroxy group, and a C₁-C₃-hydroxyalkyl group;
      a 3- to 7-membered heterocycloalkyl group, comprising one, two or three heteroatoms which are independently selected from —O— and —NR⁹—, which is optionally further substituted with a C₁-C₃-alkyl group;
      a heteroaryl group, which is optionally further substituted with a C₁-C₃-alkyl group;
   a C₃-C₇-cycloalkyl group which is optionally substituted with a hydroxy group, or a C₁-C₃-alkyl group and
   a 3- to 6-membered heterocycloalkyl group, which is optionally substituted with one or two substituents, said substituent independently selected from C₁-C₃-alkyl group and a hydroxy group;
R⁹ is a hydrogen atom or a C₁-C₃-alkyl group or a bond;
   or a stereoisomer, a tautomer, a hydrate, a solvate, or a salt thereof, or a mixture of same or the compounds mentioned above for use in the treatment of sarcoma.

In accordance with a fourth embodiment of the first aspect, the present invention includes the method of treatment of sarcoma comprising administering to a patient in need thereof a compound of general formula (I), supra, in which:
R¹ is selected from a hydrogen atom, a halogen atom, a C₁-C₃-alkyl group, a C₁-C₃-haloalkyl group, and a C₁-C₃-haloalkoxy group;
R² is selected from a hydrogen atom and a halogen atom;
R³ is selected from,
   a C₁-C₆-alkyl group which is optionally substituted with a substituent which is selected from a hydroxy group, a C₁-C₄-alkoxy group and a 3- to 7-membered heterocycloalkyl group;
   a C₂-C₆-alkenyl group which is optionally substituted with an C₁-C₄-alkoxy group;
   a C₄-C₆-cycloalkyl group, which is optionally substituted with a hydroxy group;
   a C₅-C₇-cycloalkenyl group;
   a 3- to 6-membered heterocycloalkyl group, comprising one, or two heteroatoms which are independently selected from —O—, and —NR⁹—,
      and said heterocycloalkyl group is optionally further substituted with one, or two substituents and each substituent is independently selected from
      a halogen atom;
      a cyano group;
      a hydroxy group;
      a C₁-C₃-alkyl group which is optionally further substituted with a hydroxy group;
   a 5- to 6-membered-heterocycloalkyl group, comprising a heteroatom which is selected from —O—, and —NR⁹—, which is partially unsaturated and optionally substituted with a substituent which is selected from a C₁-C₃-alkyl group and a halogen atom;
   an aryl group which is optionally substituted with one, or two, substituents and each substituent is independently selected from a halogen atom, a hydroxy group, a cyano group, a C₁-C₃-alkyl group, a C₁-C₃-haloalkyl group, and a NR⁵R⁶ group;
   a mono- or bicyclic heteroaryl group which is optionally substituted with a substituent which is selected from a halogen atom, a C₁-C₃-alkyl group a C₁-C₃-haloalkyl group, a C₁-C₃-alkoxy group, and a NR⁵R⁶ group, with the proviso that said monocyclic heteroaryl group is not a pyridin-4-yl group;
   and a NR⁷R⁸ group;
R⁴ is selected from a hydrogen atom, and a C₁-C₃-alkyl group;
R⁵/R⁶ are independently selected from a hydrogen atom and a C₁-C₆-alkyl group;
R⁷/R⁸ are independently selected from
   a hydrogen atom, with the proviso that R⁷=R⁸=hydrogen is excluded,
   a C₁-C₆-alkyl group, which is optionally substituted with one, two, three or four substituents and said substituent is independently selected from
      a halogen atom;
      a hydroxy group;
      a C₁-C₃-alkoxy group;
      a C₃-C₆-cycloalkyl group which is optionally further substituted with one or two substituents and said substituents are independently selected from a C₁-C₃-alkyl group and a C₁-C₃-hydroxyalkyl group;
      a 4- to 6-membered heterocycloalkyl group, comprising one, two or three heteroatoms which are independently selected from —O— and —NR⁹—, which is optionally further substituted with a C₁-C₃-alkyl group;
      a heteroaryl group, which is optionally further substituted with a C₁-C₃-alkyl group;
   a C₃-C₆-cycloalkyl group which is optionally substituted with a hydroxy group, or a C₁-C₃-alkyl group;
   a 3- to 6-membered heterocycloalkyl group, which is optionally substituted with one or two substituents, said substituent independently selected from C₁-C₃-alkyl group and a hydroxy group, and
R⁹ is a hydrogen atom or a C₁-C₃-alkyl group or a bond;
   or a stereoisomer, a tautomer, a hydrate, a solvate, or a salt thereof, or a mixture of same or the compounds mentioned above for use in the treatment of sarcoma.

In accordance with a fifth embodiment of the first aspect, the present invention includes the method of treatment of sarcoma comprising administering to a patient in need thereof a compound of general formula (I), supra, in which:
R¹ is selected from a hydrogen atom, a halogen atom, a C₁-C₃-alkyl group, and a C₁-C₃-haloalkyl group;
R² is selected from a hydrogen atom and a halogen atom;
R³ is selected from,
   a C₁-C₆-alkyl group which is optionally substituted with one or two substituents and each substituent is independently selected from a hydroxy group, a C₁-C₄-alkoxy group, and a 3- to 7-membered heterocycloalkyl group;
   a C₂-C₆-alkenyl group which is optionally substituted with an C₁-C₃-alkoxy group;
   a C₃-C₇-cycloalkyl group, which is optionally substituted with a hydroxy group;
   a C₅-C₆-cycloalkenyl group, which is optionally substituted with a hydroxy group;
   a 3- to 6-membered heterocycloalkyl group, comprising one, two or three heteroatoms which are independently selected from —O— and —NR⁹—,
      and said heterocycloalkyl group is optionally substituted with one, two or three substituents and each substituent is independently selected from
      a halogen atom;
      a oxo (═O) group;
      a cyano group;
      a hydroxy group;
      a C₁-C₃-alkyl group which is optionally further substituted with a hydroxy group;
   a 5- to 7-membered heterocycloalkyl group which is partially unsaturated and optionally substituted with one or two substituents and each substituent is independently selected from a C₁-C₃-alkyl group and a halogen atom;
   an aryl group which is optionally substituted with one, two, three or four substituents and each substituent is independently selected from a halogen atom, a hydroxy group, a cyano group, a C₁-C₃-alkyl group, a C₁-C₃-haloalkyl group, a C₁-C₃-alkoxy group, a C₁-C₃-haloalkoxy group, and a NR⁵R⁶ group;
   a mono- or bicyclic heteroaryl group which is optionally substituted with one or two substituents and each substituent is independently selected from a halogen atom, a C₁-C₃-alkyl group, a cyano group, a C₁-C₃-haloalkyl group, a C₁-C₃-alkoxy group, a hydroxy group, and a NR⁵R⁶ group, with the proviso that said monocyclic heteroaryl group is not a pyridin-4-yl group;
   and a NR⁷R⁸ group;
R⁴ is selected from a hydrogen atom, and a C₁-C₃-alkyl group;
R⁵/R⁶ are independently selected from a hydrogen atom and a C₁-C₆-alkyl group;
R⁷/R⁸ are independently selected from
   a hydrogen atom, with the proviso that R⁷=R⁸=hydrogen is excluded,
   a C₁-C₆-alkyl group,
   which is optionally substituted with one, two, three or four substituents and said substituent is independently selected from
      a halogen atom,
      a cyano group,
      a hydroxy group,
      a C₁-C₃-alkoxy group,
      a C₃-C₇-cycloalkyl group which is optionally substituted with one or two substituents and said substituents are independently selected from a C₁-C₃-alkyl group, a hydroxy group, and a C₁-C₃-hydroxyalkyl group;
      a 3- to 7-membered heterocycloalkyl group which itself is optionally substituted with a C₁-C₃-alkyl group;
      and a heteroaryl group, which is optionally further substituted with a C₁-C₃-alkyl group;
   a C₃-C₆-cycloalkyl group which is optionally substituted with a hydroxy group, and
   a 3- to 6-membered heterocycloalkyl group, which is optionally substituted with one or two substituents said substituent independently selected from C₁-C₃-alkyl group and a hydroxy group,
R⁹ is a hydrogen atom or a C₁-C₃-alkyl group or a bond;
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is selected from a hydrogen atom, a halogen atom, a C₁-C₃-alkyl group, and a C₁-C₃-haloalkyl group;
R² is selected from a hydrogen atom and a halogen atom;
R³ is selected from,
   a 3- to 6-membered heterocycloalkyl group, comprising one, or two heteroatoms which are independently selected from —O—, and —NR⁹—,
      and said heterocycloalkyl group is optionally further substituted with one, or two substituents and each substituent is independently selected from
      a halogen atom;
      a hydroxy group;
      and a C₁-C₃-alkyl group which is optionally further substituted with a hydroxy group;
   an aryl group which is optionally substituted with one, or two, substituents and each substituent is independently selected from a halogen atom, a hydroxy group, and a C₁-C₃-haloalkyl group;
   a mono- or bicyclic heteroaryl group which is optionally substituted with a substituent which is selected from a halogen atom, a C₁-C₃-alkyl group a C₁-C₃-haloalkyl group and a NR⁵R⁶ group, with the proviso that said monocyclic heteroaryl group is not a pyridin-4-yl group;
   and a NR⁷R⁸ group;
R⁴ is selected from a hydrogen atom, and a C₁-C₃-alkyl group;
R⁵/R⁶ are independently selected from a hydrogen atom and a C₁-C₃-alkyl group;
R⁷/R⁸ are independently selected from
   a hydrogen atom, with the proviso that R⁷=R⁸=hydrogen is excluded,
   a C₁-C₆-alkyl group, which is optionally substituted with one or two, three or four substituents and said substituent is independently selected from
      a halogen atom;
      a hydroxy group;
      a C₁-C₃-alkoxy group;
      a C₃-C₅-cycloalkyl group which is optionally further substituted with one or two substituents and said substituents are independently selected from a C₁-C₃-alkyl group and a C₁-C₃-hydroxyalkyl group;
      a 5- to 6-membered heterocycloalkyl group, comprising one, or two heteroatoms which are independently selected from —O— and —NR⁹—, which is optionally further substituted with a C₁-C₃-alkyl group,
      a heteroaryl group, which is optionally further substituted with a C₁-C₃-alkyl group;
   a C₃-C₆-cycloalkyl group which is optionally substituted with a hydroxy group, or a C₁-C₃-alkyl group;
   a 4- to 5-membered heterocycloalkyl group, which is optionally substituted with one or two substituents, said substituent independently selected from C₁-C₃-alkyl group and a hydroxy group; and
R⁹ is a hydrogen atom or a C₁-C₃-alkyl group or a bond;
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is selected from CF₃ and a fluorine atom;
R² is a hydrogen atom;
R³ is selected from
   an aryl group which is optionally substituted with a substituent which is selected from a halogen atom and a C₁-C₃-haloalkyl group,
   a monocyclic heteroaryl group substituted with a substituent which is selected from C₁-C₃-haloalkyl group and NR⁵R⁶ group;
   and a NR⁷R⁸ group;
R⁴ is selected from a hydrogen atom and a methyl group;
R⁵/R⁶ are independently selected from a hydrogen atom and a methyl group;
R⁷/R⁸ are independently selected from
   a hydrogen atom, with the proviso that R⁷=R⁸=hydrogen is excluded,
   a C₁-C₃-alkyl group, which is optionally substituted with one, two or four substituents and said substituent is independently selected from
      a halogen atom, a hydroxy group, and a methoxy group,
         or a stereoisomer, a tautomer, a hydrate, a solvate, or a salt thereof, or a mixture of same.

In accordance with a further embodiment of the first aspect, the present invention includes the method of treatment of sarcoma comprising administering to a patient in need thereof a compound of general formula (I), supra, in which:
R¹ is selected from CF₃ and a fluorine atom;
R² is a hydrogen atom;
R³ is selected from
   an aryl group which is optionally substituted with a substituent which is selected from a halogen atom and a C₁-C₃-haloalkyl group,
   a monocyclic heteroaryl group substituted with a substituent which is selected from C₁-C₃-haloalkyl group and NR⁵R⁶ group;
   and a NR⁷R⁸ group;
R⁴ is a hydrogen atom;
R⁵/R⁶ are independently selected from a hydrogen atom and a methyl group;
R⁷/R⁸ are independently selected from
   a hydrogen atom, with the proviso that R⁷=R⁸=hydrogen is excluded,
   a C₁-C₃-alkyl group, which is optionally substituted with one, two or four substituents and said substituent is independently selected from
      a halogen atom, a hydroxy group, and a methoxy group,
         or a stereoisomer, a tautomer, a hydrate, a solvate, or a salt thereof, or a mixture of same.

In accordance with a further embodiment of the first aspect, the present invention includes the method of treatment of sarcoma comprising administering to a patient in need thereof a compound of general formula (I), supra, in which:
R¹ is selected from CF₃ and a fluorine atom;
R² is a hydrogen atom;
R³ is selected from
   an aryl group which is optionally substituted with a substituent which is selected from a halogen atom and a C₁-C₃-haloalkyl group,
R⁴ is selected from a hydrogen atom;
   or a stereoisomer, a tautomer, a hydrate, a solvate, or a salt thereof, or a mixture of same.

In accordance with a further embodiment of the first aspect, the present invention includes the method of treatment of sarcoma comprising administering to a patient in need thereof a compound of general formula (I), supra, in which:
R¹ is selected from CF₃ and a fluorine atom;
R² is a hydrogen atom;
R³ is selected from
   a monocyclic heteroaryl group substituted with a substituent which is selected from C₁-C₃-haloalkyl group and NR⁵R⁶ group;
   and a NR⁷R⁸ group;
R⁴ is selected from a hydrogen atom;
R⁵/R⁶ are independently selected from a hydrogen atom and a methyl group;
R⁷/R⁸ are independently selected from
   a hydrogen atom, with the proviso that R⁷=R⁸=hydrogen is excluded,
   a C₁-C₃-alkyl group, which is optionally substituted with one, two or four substituents and said substituent is independently selected from
      a halogen atom, a hydroxy group, and a methoxy group,
         or a stereoisomer, a tautomer, a hydrate, a solvate, or a salt thereof, or a mixture of same.

In accordance with a further embodiment of the first aspect, the present invention includes the method of treatment of sarcoma comprising administering to a patient in need thereof a compound of general formula (I), supra, in which:
R¹ is selected from CF₃ and a fluorine atom;
R² is a hydrogen atom;
R³ is selected from
   an aryl group which is optionally substituted with a substituent which is selected from a halogen atom and a C₁-C₃-haloalkyl group,
   a monocyclic heteroaryl group substituted with a substituent which is selected from C₁-C₃-haloalkyl group and NR⁵R⁶ group;
   and a NR⁷R⁸ group;
R⁴ is a methyl group;
R⁵/R⁶ are independently selected from a hydrogen atom and a methyl group;
R⁷/R⁸ are independently selected from
   a hydrogen atom, with the proviso that R⁷=R⁸=hydrogen is excluded,
   a C₁-C₃-alkyl group, which is optionally substituted with one, two or four substituents and said substituent is independently selected from
      a halogen atom, a hydroxy group, and a methoxy group,
         or a stereoisomer, a tautomer, a hydrate, a solvate, or a salt thereof, or a mixture of same.

In accordance with a further embodiment of the first aspect, the present invention includes the method of treatment of sarcoma comprising administering to a patient in need thereof a compound of general formula (I), supra, in which:
R¹ is selected from CF₃ and a fluorine atom;
R² is a hydrogen atom;
R³ is selected from
   an aryl group which is optionally substituted with a substituent which is selected from a halogen atom and a C₁-C₃-haloalkyl group,
R⁴ is a methyl group;
   or a stereoisomer, a tautomer, a hydrate, a solvate, or a salt thereof, or a mixture of same.

In accordance with a further embodiment of the first aspect, the present invention includes the method of treatment of sarcoma comprising administering to a patient in need thereof a compound of general formula (I), supra, in which:
R¹ is selected from CF₃ and a fluorine atom;
R² is a hydrogen atom;
R³ is selected from
   a monocyclic heteroaryl group substituted with a substituent which is selected from C₁-C₃-haloalkyl group and NR⁵R⁶ group;
   and a NR⁷R⁸ group;
R⁴ is a methyl group;
R⁵/R⁶ are independently selected from a hydrogen atom and a methyl group;
R⁷/R⁸ are independently selected from
   a hydrogen atom, with the proviso that R⁷=R⁸=hydrogen is excluded,
   a C₁-C₃-alkyl group, which is optionally substituted with one, two or four substituents and said substituent is independently selected from
      a halogen atom, a hydroxy group, and a methoxy group,
         or a stereoisomer, a tautomer, a hydrate, a solvate, or a salt thereof, or a mixture of same.

In accordance with a further embodiment of the first aspect, the present invention includes the method of treatment of sarcoma comprising administering to a patient in need thereof a compound of general formula (I), supra, in which:
R¹ is selected from a C₁-C₃-alkyl group (e.g. CH₃ group), a C₁-C₃-haloalkyl group (e.g. CF₃ group) and a halogen (e.g., fluorine atom);
R² is a hydrogen atom;
R³ is selected from
   a C₁-C₆-alkyl group, which is optionally substituted with a C₁-C₄-alkoxy group (e.g., methoxy) group
   a 6-membered heterocycloalkyl group, comprising one, or two heteroatoms which are independently selected from —O— and —NR⁹—,
      and said heterocycloalkyl group is optionally further substituted with one, or two substituents and each substituent is independently selected from
      a halogen atom and a C₁-C₃-alkyl group,
   a phenyl group, which is substituted with one or two substituents selected from a halogen atom or a C₁-C₃-haloalkyl group,
   a monocyclic 5-membered heteroaryl group which is substituted with a substituent which is selected from a C₁-C₃-haloalkyl group;
R⁴ is selected from a hydrogen atom and a C₁-C₃-alkyl group (e.g. methyl group);
R⁹ is a bond or C₁-C₃-alkyl group (e.g., methyl),
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is selected from a C₁-C₃-alkyl group (e.g. CH₃ group), a C₁-C₃-haloalkyl group (e.g. CF₃ group) and a halogen (e.g., fluorine atom);
R² is a hydrogen atom;
R³ is selected from
   a C₁-C₆-alkyl group, which is optionally substituted with a C₁-C₄-alkoxy group (e.g., methoxy) group
   a 6-membered heterocycloalkyl group, comprising one, or two heteroatoms which are independently selected from —O— and —NR⁹—,
      and said heterocycloalkyl group is optionally further substituted with one, or two substituents and each substituent is independently selected from
      a halogen atom and a C₁-C₃-alkyl group,
   a phenyl group, which is substituted with one or two substituents selected from a halogen atom or a C₁-C₃-haloalkyl group,
   a monocyclic 5-membered heteroaryl group which is substituted with a substituent which is selected from a C₁-C₃-haloalkyl group;
R⁴ is a hydrogen atom;
R⁹ is a bond or C₁-C₃-alkyl group (e.g., methyl),
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is selected from a C₁-C₃-alkyl group (e.g. CH₃ group), a C₁-C₃-haloalkyl group (e.g. CF₃ group) and a halogen (e.g., fluorine atom);
R² is a hydrogen atom;
R³ is selected from
   a C₁-C₆-alkyl group, which is optionally substituted with a C₁-C₄-alkoxy group (e.g., methoxy) group
   a 6-membered heterocycloalkyl group, comprising one, or two heteroatoms which are independently selected from —O— and —NR⁹—,
      and said heterocycloalkyl group is optionally further substituted with one, or two substituents and each substituent is independently selected from
      a halogen atom and a C₁-C₃-alkyl group,
   a phenyl group, which is substituted with one or two substituents selected from a halogen atom or a C₁-C₃-haloalkyl group,
   a monocyclic 5-membered heteroaryl group which is substituted with a substituent which is selected from a C₁-C₃-haloalkyl group;
R⁴ is a C₁-C₃-alkyl group (e.g. methyl group);
R⁹ is a bond or C₁-C₃-alkyl group (e.g., methyl),
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is selected from a C₁-C₃-alkyl group (e.g. CH₃ group), a C₁-C₃-haloalkyl group (e.g. CF₃ group) and a halogen (e.g., fluorine atom);
R² is a hydrogen atom;
R³ is selected from
   a halogen atom,
   a C₁-C₆-alkyl group, which is optionally substituted with a C₁-C₄-alkoxy group (e.g., methoxy) group
   a 6-membered heterocycloalkyl group, comprising one, or two heteroatoms which are independently selected from —O— and —NR⁹—,
      and said heterocycloalkyl group is optionally further substituted with one, or two substituents and each substituent is independently selected from
      a halogen atom and a C₁-C₃-alkyl group,
   a phenyl group, which is substituted with one or two substituents selected from a halogen atom or a C₁-C₃-haloalkyl group,
   a monocyclic 5-membered heteroaryl group which is substituted with a substituent which is selected from a C₁-C₃-haloalkyl group;
R⁴ is selected from a hydrogen atom and a C₁-C₃-alkyl group (e.g. methyl group);
R⁹ is a C₁-C₃-alkyl group (e.g., methyl),
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is selected from a CH₃ group, a CF₃ group and a fluorine atom;
R² is a hydrogen atom;
R³ is selected from
   a fluorine atom,
   a —(CH₂)₂C(CH₃)₃ group, a —(CH₂)₃—OCH₃ group,
   a N-methyl-piperidin- group, a morpholino group, a 4,4-difluoropiperidin-1-yl group,
   a phenyl group, which is substituted with one or two substituents independently selected from a fluorine atom and a difluoromethyl group,
   a pyrazol group which is substituted with a difluoromethyl group or a trifluoromethyl group;
R⁴ is selected from a hydrogen atom and a methyl group;
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is selected from a CH₃ group, a CF₃ group and a fluorine atom;
R² is a hydrogen atom;
R³ is selected from
   a —CH₃ group, a —(CH₂)₂C(CH₃)₃ group, a —(CH₂)₃—OCH₃ group,
   a N-methyl-piperazin- group, a morpholino group, a 4,4-difluoropiperidin-1-yl group,
   a phenyl group, which is substituted with one or two substituents independently selected from a fluorine atom and a difluoromethyl group,
   a pyrazol group which is substituted with a difluoromethyl group or a trifluoromethyl group;
R⁴ is selected from a hydrogen atom and a methyl group;
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is a C₁-C₃-haloalkyl group;
R² is a hydrogen atom;
R³ is selected from
   a 6-membered heterocycloalkyl group, comprising one, or two heteroatoms which are independently selected from —NR⁹—,
   a monocyclic 5-membered heteroaryl group which is substituted with a substituent which is selected from a C₁-C₃-haloalkyl group and
   a phenyl group, which is substituted a halogen atom,
R⁴ is a methyl group;
R⁹ is a bond or a C₁-C₃-alkyl group
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is a CF₃ group;
R² is a hydrogen atom;
R³ is selected from
   a N-methyl-piperazin- group, a trifluoromethylpyrazol group and a phenyl group, which is substituted with a fluorine atom,
R⁴ is a methyl group;
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is a CF₃ group;
R² is a hydrogen atom;
R³ is selected from
   a N-methyl-piperidin- group or a phenyl group, which is substituted a fluorine atom,
R⁴ is a methyl group;
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is selected from a CH₃ group, a CF₃ group and a fluorine atom;
R² is a hydrogen atom;
R³ is selected from
   a fluorine atom,
   a —(CH₂)₂C(CH₃)₃ group, a —(CH₂)₃—OCH₃ group,
   a morpholino group, a 4,4-difluoropiperidin-1-yl group, a
   a phenyl group, which is substituted with one or two substituents independently selected from a fluorine atom and a difluoromethyl group,
   a pyrazol group which is substituted with a difluoromethyl group or a trifluoromethyl group;
R⁴ is a hydrogen atom;
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is selected from a CH₃ group, a CF₃ group and a fluorine atom;
R² is a hydrogen atom;
R³ is selected from
   a —CH₃ group, a —(CH₂)₂C(CH₃)₃ group, a —(CH₂)₃—OCH₃ group,
   a morpholino group, a 4,4-difluoropiperidin-1-yl group,
   a phenyl group, which is substituted with one or two substituents independently selected from a fluorine atom and a difluoromethyl group,
   a pyrazol group which is substituted with a difluoromethyl group or a trifluoromethyl group;
R⁴ is a hydrogen atom or a —CH₃ group;
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, in which:
R¹ is selected from a CH₃ group, a CF₃ group and a fluorine atom;
R² is a hydrogen atom;
R³ is selected from
   a CH₃ group, a —(CH₂)₂C(CH₃)₃ group, a —(CH₂)₃—OCH₃ group,
   a morpholino group, a 4,4-difluoropiperidin-1-yl group, a
   a phenyl group, which is substituted with one or two substituents independently selected from a fluorine atom and a difluoromethyl group,
   a pyrazol group which is substituted with a difluoromethyl group;
R⁴ is a hydrogen atom;
   or a stereoisomer, a tautomer, a hydrate, a solvate, or a salt thereof, or a mixture of same or the compounds mentioned above for use in the treatment of sarcoma.

In accordance with a further embodiment of the first aspect, the present invention includes the method of treatment of sarcoma comprising administering to a patient in need thereof a compound of general formula (I), supra, which is selected from the group:
5-[4-(4,4-difluoropiperidin-1-yl)-3-fluorophenyl]-3,6-dihydro-2H-1,3,4-oxadiazin-2-one,
5-[4-chloro-3-(trifluoromethyl)phenyl]-3,6-dihydro-2H-1,3,4-oxadiazin-2-one,
5-[4′-fluoro-2-(trifluoromethyl)biphenyl-4-yl]-3,6-dihydro-2H-1,3,4-oxadiazin-2-one,
5-[3-fluoro-4-(morpholin-4-yl)phenyl]-3,6-dihydro-2H-1,3,4-oxadiazin-2-one,
5-[3-fluoro-4-(4-fluoro-4-methylpiperidin-1-yl)phenyl]-3,6-dihydro-2H-1,3,4-oxadiazin-2-one,
5-[3-fluoro-4-(4-fluoropiperidin-1-yl)phenyl]-3,6-dihydro-2H-1,3,4-oxadiazin-2-one,
5-(4′-fluoro-2-methylbiphenyl-4-yl)-3,6-dihydro-2H-1,3,4-oxadiazin-2-one,
5-(3′,4′-difluoro-2-methylbiphenyl-4-yl)-3,6-dihydro-2H-1,3,4-oxadiazin-2-one,
5-(4′-fluoro-2,2′-dimethylbiphenyl-4-yl)-3,6-dihydro-2H-1,3,4-oxadiazin-2-one,
5-[4-(3,6-dihydro-2H-pyran-4-yl)-3-methylphenyl]-3,6-dihydro-2H-1,3,4-oxadiazin-2-one,
5-[3-methyl-4-(1H-pyrazol-4-yl)phenyl]-3,6-dihydro-2H-1,3,4-oxadiazin-2-one,
5-[3-methyl-4-(6-oxo-1,6-dihydropyridin-3-yl)phenyl]-3,6-dihydro-2H-1,3,4-oxadiazin-2-one,
5-[3-methyl-4-(pyridin-3-yl)phenyl]-3,6-dihydro-2H-1,3,4-oxadiazin-2-one,
5-[3-methyl-4-(pyrimidin-5-yl)phenyl]-3,6-dihydro-2H-1,3,4-oxadiazin-2-one,
5-(3′-fluoro-2-methylbiphenyl-4-yl)-3,6-dihydro-2H-1,3,4-oxadiazin-2-one,
(rac)-6-methyl-5-(3,4,5-trifluorophenyl)-3,6-dihydro-2H-1,3,4-oxadiazin-2-one,
(rac)-5-[3,5-difluoro-4-(morpholin-4-yl)phenyl]-6-methyl-3,6-dihydro-2H-1,3,4-oxadiazin-2-one,
(rac)-5-{3,5-difluoro-4-[(2S)-2-methylmorpholin-4-yl]phenyl}-6-methyl-3,6-dihydro-2H-1,3,4-oxadiazin-2-one,
(rac)-5-(4-bromophenyl)-6-methyl-3,6-dihydro-2H-1,3,4-oxadiazin-2-one
(rac)-6-methyl-5-[4-(morpholin-4-yl)phenyl]-3,6-dihydro-2H-1,3,4-oxadiazin-2-one,
5-[3,5-difluoro-4-(morpholin-4-yl)phenyl]-3,6-dihydro-2H-1,3,4-oxadiazin-2-one,
2-(morpholin-4-yl)-5-(2-oxo-3,6-dihydro-2H-1,3,4-oxadiazin-5-yl)benzonitrile,
3-chloro-2-(morpholin-4-yl)-5-(2-oxo-3,6-dihydro-2H-1,3,4-oxadiazin-5-yl)benzonitrile,
5-{4-[2,6-dimethylmorpholin-4-yl]-3-fluorophenyl}-3,6-dihydro-2H-1,3,4-oxadiazin-2-one,
(6S)-5-(3-Fluoro-4-morpholinophenyl)-6-methyl-3,6-dihydro-2H-1,3,4-oxadiazin-2-one,
(6S)-5-(3,5-Difluoro-4-morpholinophenyl)-6-methyl-3,6-dihydro-2H-1,3,4-oxadiazin-2-one,
5-[4-(3,3-difluoropyrrolidin-1-yl)-3-fluorophenyl]-3,6-dihydro-2H-1,3,4-oxadiazin-2-one,
5-(2-methylbiphenyl-4-yl)-3,6-dihydro-2H-1,3,4-oxadiazin-2-one,
5-[3-methyl-4-(2-methylpyrimidin-5-yl)phenyl]-3,6-dihydro-2H-1,3,4-oxadiazin-2-one,
5-[3-methyl-4-(1-methyl-1H-pyrazol-5-yl)phenyl]-3,6-dihydro-2H-1,3,4-oxadiazin-2-one,
5-(2,4′-difluorobiphenyl-4-yl)-3,6-dihydro-2H-1,3,4-oxadiazin-2-one,
5-[4′-chloro-2-(trifluoromethyl)biphenyl-4-yl]-3,6-dihydro-2H-1,3,4-oxadiazin-2-one,
5-[4-(6-methylpyridin-3-yl)-3-(trifluoromethyl)phenyl]-3,6-dihydro-2H-1,3,4-oxadiazin-2-one,
5-[4-(pyridin-3-yl)-3-(trifluoromethyl)phenyl]-3,6-dihydro-2H-1,3,4-oxadiazin-2-one,
5-[4′-amino-2-(trifluoromethyl)biphenyl-4-yl]-3,6-dihydro-2H-1,3,4-oxadiazin-2-one,
5-[3′-hydroxy-4′-methyl-2-(trifluoromethyl)biphenyl-4-yl]-3,6-dihydro-2H-1,3,4-oxadiazin-2-one,
5-{3-(trifluoromethyl)-4-[6-(trifluoromethyl)pyridin-3-yl]phenyl}-3,6-dihydro-2H-1,3,4-oxadiazin-2-one,
5-[4′-fluoro-3′-hydroxy-2-(trifluoromethyl)biphenyl-4-yl]-3,6-dihydro-2H-1,3,4-oxadiazin-2-one,
5-[5′-amino-2′,4′-difluoro-2-(trifluoromethyl)biphenyl-4-yl]-3,6-dihydro-2H-1,3,4-oxadiazin-2-one,
5-[4′-amino-3′-fluoro-2-(trifluoromethyl)biphenyl-4-yl]-3,6-dihydro-2H-1,3,4-oxadiazin-2-one,
5-[4-(6-aminopyridin-3-yl)-3-(trifluoromethyl)phenyl]-3,6-dihydro-2H-1,3,4-oxadiazin-2-one,
5-[3′-amino-4′-chloro-2-(trifluoromethyl)biphenyl-4-yl]-3,6-dihydro-2H-1,3,4-oxadiazin-2-one,
5-[3′-amino-4′-methyl-2-(trifluoromethyl)biphenyl-4-yl]-3,6-dihydro-2H-1,3,4-oxadiazin-2-one,
5-[3′-amino-2′-fluoro-2-(trifluoromethyl)biphenyl-4-yl]-3,6-dihydro-2H-1,3,4-oxadiazin-2-one,
5-[4′-amino-2′-fluoro-2-(trifluoromethyl)biphenyl-4-yl]-3,6-dihydro-2H-1,3,4-oxadiazin-2-one,
5-[4-(1-methyl-1H-pyrazol-4-yl)-3-(trifluoromethyl)phenyl]-3,6-dihydro-2H-1,3,4-oxadiazin-2-one,
5-[4-(3-methyl-1H-pyrazol-4-yl)-3-(trifluoromethyl)phenyl]-3,6-dihydro-2H-1,3,4-oxadiazin-2-one,
5-[4-(1H-indazol-6-yl)-3-(trifluoromethyl)phenyl]-3,6-dihydro-2H-1,3,4-oxadiazin-2-one,
5-[4-(5-fluoro-6-methylpyridin-3-yl)-3-(trifluoromethyl)phenyl]-3,6-dihydro-2H-1,3,4-oxadiazin-2-one,
5-[4-(1,2-thiazol-4-yl)-3-(trifluoromethyl)phenyl]-3,6-dihydro-2H-1,3,4-oxadiazin-2-one,
1-methyl-5-[4-(2-oxo-3,6-dihydro-2H-1,3,4-oxadiazin-5-yl)-2-(trifluoromethyl)phenyl]-1H-pyrrole-2-carbonitrile,
5-[2,4′-bis(trifluoromethyl)biphenyl-4-yl]-3,6-dihydro-2H-1,3,4-oxadiazin-2-one,
5-[4-(1,3-dimethyl-1H-pyrazol-4-yl)-3-(trifluoromethyl)phenyl]-3,6-dihydro-2H-1,3,4-oxadiazin-2-one,
5-[4-(2-methoxy-6-methylpyridin-3-yl)-3-(trifluoromethyl)phenyl]-3,6-dihydro-2H-1,3,4-oxadiazin-2-one,
5-[4-(2-methyl-1,3-thiazol-5-yl)-3-(trifluoromethyl)phenyl]-3,6-dihydro-2H-1,3,4-oxadiazin-2-one,
5-[4′-(methylamino)-2-(trifluoromethyl)biphenyl-4-yl]-3,6-dihydro-2H-1,3,4-oxadiazin-2-one,
5-[3′-amino-4′-fluoro-2-(trifluoromethyl)biphenyl-4-yl]-3,6-dihydro-2H-1,3,4-oxadiazin-2-one,
5-[4-fluoro-3-(trifluoromethyl)phenyl]-3,6-dihydro-2H-1,3,4-oxadiazin-2-one,
5-[3′,4′,5′-trifluoro-2-(trifluoromethyl)biphenyl-4-yl]-3,6-dihydro-2H-1,3,4-oxadiazin-2-one,
5-[2′,5′-difluoro-2-(trifluoromethyl)biphenyl-4-yl]-3,6-dihydro-2H-1,3,4-oxadiazin-2-one,
5-[3′-amino-4′-fluoro-2-(trifluoromethoxy)biphenyl-4-yl]-3,6-dihydro-2H-1,3,4-oxadiazin-2-one,
5-[3′,4′-difluoro-2-(trifluoromethyl)biphenyl-4-yl]-3,6-dihydro-2H-1,3,4-oxadiazin-2-one,
5-[4-(1H-indol-6-yl)-3-(trifluoromethyl)phenyl]-3,6-dihydro-2H-1,3,4-oxadiazin-2-one,
5-[4-(2-methylprop-1-en-1-yl)-3-(trifluoromethyl)phenyl]-3,6-dihydro-2H-1,3,4-oxadiazin-2-one,
5-[2′,3′-difluoro-2-(trifluoromethyl)biphenyl-4-yl]-3,6-dihydro-2H-1,3,4-oxadiazin-2-one,
5-[4-(morpholin-4-yl)-3-(trifluoromethyl)phenyl]-3,6-dihydro-2H-1,3,4-oxadiazin-2-one,
5-[4-(butylamino)-3-(trifluoromethyl)phenyl]-3,6-dihydro-2H-1,3,4-oxadiazin-2-one,
5-[4-(ethylamino)-3-(trifluoromethyl)phenyl]-3,6-dihydro-2H-1,3,4-oxadiazin-2-one,
5-[4-(1-methyl-1H-pyrazol-4-yl)-3-(trifluoromethoxy)phenyl]-3,6-dihydro-2H-1,3,4-oxadiazin-2-one,
5-[4-(propylamino)-3-(trifluoromethyl)phenyl]-3,6-dihydro-2H-1,3,4-oxadiazin-2-one,
5-[4-(6-methylpyridin-3-yl)-3-(trifluoromethoxy)phenyl]-3,6-dihydro-2H-1,3,4-oxadiazin-2-one,
5-[4′-chloro-2-(trifluoromethoxy)biphenyl-4-yl]-3,6-dihydro-2H-1,3,4-oxadiazin-2-one,
5-[4-(azetidin-1-yl)-3-(trifluoromethyl)phenyl]-3,6-dihydro-2H-1,3,4-oxadiazin-2-one,
5-[4-(1-methyl-1H-benzimidazol-6-yl)-3-(trifluoromethyl)phenyl]-3,6-dihydro-2H-1,3,4-oxadiazin-2-one,
5-[4-(pentylamino)-3-(trifluoromethyl)phenyl]-3,6-dihydro-2H-1,3,4-oxadiazin-2-one,
5-[4-(1-methyl-1H-indazol-6-yl)-3-(trifluoromethyl)phenyl]-3,6-dihydro-2H-1,3,4-oxadiazin-2-one,
5-[4′-fluoro-2-(trifluoromethoxy)biphenyl-4-yl]-3,6-dihydro-2H-1,3,4-oxadiazin-2-one,
5-[3-fluoro-4-(6-fluoropyridin-3-yl)phenyl]-3,6-dihydro-2H-1,3,4-oxadiazin-2-one,
5-[3-fluoro-4-(3-methylpyridin-4-yl)phenyl]-3,6-dihydro-2H-1,3,4-oxadiazin-2-one,
5-[3-fluoro-4-(2-methylpyridin-4-yl)phenyl]-3,6-dihydro-2H-1,3,4-oxadiazin-2-one,
5-(4′-amino-2-fluorobiphenyl-4-yl)-3,6-dihydro-2H-1,3,4-oxadiazin-2-one,
5-(2-fluoro-2′-methylbiphenyl-4-yl)-3,6-dihydro-2H-1,3,4-oxadiazin-2-one,
5-(2′-chloro-2,4′-difluorobiphenyl-4-yl)-3,6-dihydro-2H-1,3,4-oxadiazin-2-one,
5-[4-(cyclopent-1-en-1-yl)-3-fluorophenyl]-3,6-dihydro-2H-1,3,4-oxadiazin-2-one,
5-(2′-ethyl-2-fluorobiphenyl-4-yl)-3,6-dihydro-2H-1,3,4-oxadiazin-2-one,
5-[3-fluoro-4-(6-methoxypyridin-3-yl)phenyl]-3,6-dihydro-2H-1,3,4-oxadiazin-2-one,
5-(2,4′-difluoro-3′-methylbiphenyl-4-yl)-3,6-dihydro-2H-1,3,4-oxadiazin-2-one,
5-(2-fluoro-3′-methylbiphenyl-4-yl)-3,6-dihydro-2H-1,3,4-oxadiazin-2-one,
5-(2-fluoro-4′-methylbiphenyl-4-yl)-3,6-dihydro-2H-1,3,4-oxadiazin-2-one,
5-(2-fluorobiphenyl-4-yl)-3,6-dihydro-2H-1,3,4-oxadiazin-2-one,
5-[4-(2-aminopyridin-4-yl)-3-fluorophenyl]-3,6-dihydro-2H-1,3,4-oxadiazin-2-one,
5-(3′-amino-2-fluorobiphenyl-4-yl)-3,6-dihydro-2H-1,3,4-oxadiazin-2-one,
5-[4′-(difluoromethyl)-2-fluorobiphenyl-4-yl]-3,6-dihydro-2H-1,3,4-oxadiazin-2-one,
5-[3-fluoro-4-(pyridin-4-yl)phenyl]-3,6-dihydro-2H-1,3,4-oxadiazin-2-one,
5-[3-fluoro-4-(2-methylpyrimidin-5-yl)phenyl]-3,6-dihydro-2H-1,3,4-oxadiazin-2-one,
5-[3-fluoro-4-(2-methoxypyridin-4-yl)phenyl]-3,6-dihydro-2H-1,3,4-oxadiazin-2-one,
5-[3-fluoro-4-(2-methylpyridin-3-yl)phenyl]-3,6-dihydro-2H-1,3,4-oxadiazin-2-one,
5-[3-fluoro-4-(6-methylpyridin-3-yl)phenyl]-3,6-dihydro-2H-1,3,4-oxadiazin-2-one,
5-(2,2′,4′,5′-tetrafluorobiphenyl-4-yl)-3,6-dihydro-2H-1,3,4-oxadiazin-2-one,
5-(2,2′,3′,4′-tetrafluorobiphenyl-4-yl)-3,6-dihydro-2H-1,3,4-oxadiazin-2-one,
5-(2,2′,5′-trifluorobiphenyl-4-yl)-3,6-dihydro-2H-1,3,4-oxadiazin-2-one,
2′-fluoro-4′-(2-oxo-3,6-dihydro-2H-1,3,4-oxadiazin-5-yl)biphenyl-4-carbonitrile,
5-(2′-amino-2-fluorobiphenyl-4-yl)-3,6-dihydro-2H-1,3,4-oxadiazin-2-one,
5-(3′-amino-2-fluoro-4′-methylbiphenyl-4-yl)-3,6-dihydro-2H-1,3,4-oxadiazin-2-one,
5-(2-fluoro-3′-hydroxybiphenyl-4-yl)-3,6-dihydro-2H-1,3,4-oxadiazin-2-one,
5-(2-fluoro-4′-hydroxybiphenyl-4-yl)-3,6-dihydro-2H-1,3,4-oxadiazin-2-one,
5-(2-fluoro-2′-hydroxybiphenyl-4-yl)-3,6-dihydro-2H-1,3,4-oxadiazin-2-one,
5-(2,3′,4′-trifluorobiphenyl-4-yl)-3,6-dihydro-2H-1,3,4-oxadiazin-2-one,
5-[3-fluoro-4-(pyridin-3-yl)phenyl]-3,6-dihydro-2H-1,3,4-oxadiazin-2-one,
5-(2,2′,3′-trifluorobiphenyl-4-yl)-3,6-dihydro-2H-1,3,4-oxadiazin-2-one,
5-(2,3′,5′-trifluorobiphenyl-4-yl)-3,6-dihydro-2H-1,3,4-oxadiazin-2-one,
5-(2,2′,4′-trifluorobiphenyl-4-yl)-3,6-dihydro-2H-1,3,4-oxadiazin-2-one,
5-(2-fluoro-2′,4′-dimethylbiphenyl-4-yl)-3,6-dihydro-2H-1,3,4-oxadiazin-2-one,
5-(2,3′-difluoro-4′-methylbiphenyl-4-yl)-3,6-dihydro-2H-1,3,4-oxadiazin-2-one,
5-(2,2′-difluorobiphenyl-4-yl)-3,6-dihydro-2H-1,3,4-oxadiazin-2-one,
5-(2,2′,6′-trifluorobiphenyl-4-yl)-3,6-dihydro-2H-1,3,4-oxadiazin-2-one,
5-(2-fluoro-2′-methoxybiphenyl-4-yl)-3,6-dihydro-2H-1,3,4-oxadiazin-2-one,
5-(2,3′-difluorobiphenyl-4-yl)-3,6-dihydro-2H-1,3,4-oxadiazin-2-one,
5-[3-fluoro-4-(4-methylpyridin-3-yl)phenyl]-3,6-dihydro-2H-1,3,4-oxadiazin-2-one,
(rac)-5-(3-Fluoro-4-morpholinophenyl)-6-methyl-3,6-dihydro-2H-1,3,4-oxadiazin-2-one,
(6S)-6-methyl-5-[4-(morpholin-4-yl)-3-(trifluoromethyl)phenyl]-3,6-dihydro-2H-1,3,4-oxadiazin-2-one,
(6S)-5-(-[(3-chloro-4-(morpholin-4-yl)-5-(trifluoromethyl)phenyl)-)]-6-methyl-3,6-dihydro-2H-1,3,4-oxadiazin-2-one,
6S)-5-(-[(4-chloro-3-(trifluoromethyl)phenyl)-)]-6-methyl-3,6-dihydro-2H-1,3,4-oxadiazin-2-one,
(6S)-5-(-[(4-fluoro-3-(trifluoromethyl)phenyl)-)]-6-methyl-3,6-dihydro-2H-1,3,4-oxadiazin-2-one,
5-[4-chloro-3-(trifluoromethoxy)phenyl]-3,6-dihydro-2H-1,3,4-oxadiazin-2-one,
5-(4-chloro-3-methylphenyl)-3,6-dihydro-2H-1,3,4-oxadiazin-2-one,
(rac)-6-methyl-5-(4-morpholino-3-(trifluoromethyl)phenyl)-3,6-dihydro-2H-1,3,4-oxadiazin-2-one,
5-{4-[1-methyl-3-(trifluoromethyl)-1H-pyrazol-4-yl]-3-(trifluoromethyl)phenyl}-3,6-dihydro-2H-1,3,4-oxadiazin-2-one,
5-[4-(3,5-dimethyl-1H-pyrazol-4-yl)-3-(trifluoromethyl)phenyl]-3,6-dihydro-2H-1,3,4-oxadiazin-2-one,
5-[4-(3,5-dimethyl-1,2-oxazol-4-yl)-3-(trifluoromethyl)phenyl]-3,6-dihydro-2H-1,3,4-oxadiazin-2-one,
5-{3-(trifluoromethyl)-4-[3-(trifluoromethyl)-1H-pyrazol-4-yl]phenyl}-3,6-dihydro-2H-1,3,4-oxadiazin-2-one,
5-[4-(1-ethyl-1H-pyrazol-4-yl)-3-(trifluoromethyl)phenyl]-3,6-dihydro-2H-1,3,4-oxadiazin-2-one,
5-{4-[cyclopentyl(methyl)amino]-3-(trifluoromethyl)phenyl}-3,6-dihydro-2H-1,3,4-oxadiazin-2-one,
5-[4-(1-methyl-1,2,3,6-tetrahydropyridin-4-yl)-3-(trifluoromethyl)phenyl]-3,6-dihydro-2H-1,3,4-oxadiazin-2-one,
5-{4-[butyl(methyl)amino]-3-(trifluoromethyl)phenyl}-3,6-dihydro-2H-1,3,4-oxadiazin-2-one,
(6S)-5-[4′-fluoro-2-(trifluoromethyl)biphenyl-4-yl]-6-methyl-3,6-dihydro-2H-1,3,4-oxadiazin-2-one,
5-[4-(cyclopentylamino)-3-(trifluoromethyl)phenyl]-3,6-dihydro-2H-1,3,4-oxadiazin-2-one,
5-[4-(cyclopentylamino)-3-(trifluoromethyl)phenyl]-3,6-dihydro-2H-1,3,4-oxadiazin-2-one,
5-[3′-fluoro-2-(trifluoromethyl)biphenyl-4-yl]-3,6-dihydro-2H-1,3,4-oxadiazin-2-one,
5-[4-methyl-3-(trifluoromethyl)phenyl]-3,6-dihydro-2H-1,3,4-oxadiazin-2-one,
5-{4-[3-methoxyprop-1-en-1-yl]-3-(trifluoromethyl)phenyl}-3,6-dihydro-2H-1,3,4-oxadiazin-2-one,
(rac)-5-[4′-hydroxy-2-(trifluoromethyl)-2′,3′,4′,5′-tetrahydro[1,1′-biphenyl]-4-yl]-3,6-dihydro-2H-1,3,4-oxadiazin-2-one,
5-[4-(5,6-dihydro-2H-pyran-3-yl)-3-(trifluoromethyl)phenyl]-3,6-dihydro-2H-1,3,4-oxadiazin-2-one,
5-[4-(imidazo[1,2-a]pyridin-6-yl)-3-(trifluoromethyl)phenyl]-3,6-dihydro-2H-1,3,4-oxadiazin-2-one,
5-{4-[3,3-dimethylbut-1-en-1-yl]-3-(trifluoromethyl)phenyl}-3,6-dihydro-2H-1,3,4-oxadiazin-2-one,
5-{3-(trifluoromethyl)-4-[5-(trifluoromethyl)thiophen-3-yl]phenyl}-3,6-dihydro-2H-1,3,4-oxadiazin-2-one,
5-{4-[1-(difluoromethyl)-1H-pyrazol-4-yl]-3-(trifluoromethyl)phenyl}-3,6-dihydro-2H-1,3,4-oxadiazin-2-one,
5-[4-(prop-1-en-2-yl)-3-(trifluoromethyl)phenyl]-3,6-dihydro-2H-1,3,4-oxadiazin-2-one,
5-[4-(1-benzothiophen-2-yl)-3-(trifluoromethyl)phenyl]-3,6-dihydro-2H-1,3,4-oxadiazin-2-one,
5-[4-(2,5-dihydrofuran-3-yl)-3-(trifluoromethyl)phenyl]-3,6-dihydro-2H-1,3,4-oxadiazin-2-one,
5-[4-(cyclopent-1-en-1-yl)-3-(trifluoromethyl)phenyl]-3,6-dihydro-2H-1,3,4-oxadiazin-2-one,
5-[4-(1-ethyl-1H-imidazol-4-yl)-3-(trifluoromethyl)phenyl]-3,6-dihydro-2H-1,3,4-oxadiazin-2-one,
3-methyl-5-[4-(2-oxo-3,6-dihydro-2H-1,3,4-oxadiazin-5-yl)-2-(trifluoromethyl)phenyl]thiophene-2-carbonitrile,
5-{4-[1-(propan-2-yl)-1H-pyrazol-4-yl]-3-(trifluoromethyl)phenyl}-3,6-dihydro-2H-1,3,4-oxadiazin-2-one,
(rac)-5-[4-(bicyclo[2.2.1]hept-2-en-2-yl)-3-(trifluoromethyl)phenyl]-3,6-dihydro-2H-1,3,4-oxadiazin-2-one,
5-[2′-fluoro-2-(trifluoromethyl)[1,1′-biphenyl]-4-yl]-3,6-dihydro-2H-1,3,4-oxadiazin-2-one,
5-{3-(Trifluoromethyl)-4-[5-(trifluoromethyl)thiophen-2-yl]phenyl}-3,6-dihydro-2H-1,3,4-oxadiazin-2-one,
5-[4-(5-methylpyridin-2-yl)-3-(trifluoromethyl)phenyl]-3,6-dihydro-2H-1,3,4-oxadiazin-2-one,
5-[4-(5-fluoropyridin-2-yl)-3-(trifluoromethyl)phenyl]-3,6-dihydro-2H-1,3,4-oxadiazin-2-one,
5-[4-(5-chloropyridin-2-yl)-3-(trifluoromethyl)phenyl]-3,6-dihydro-2H-1,3,4-oxadiazin-2-one,
5-[4-(pyridin-2-yl)-3-(trifluoromethyl)phenyl]-3,6-dihydro-2H-1,3,4-oxadiazin-2-one,
5-[2′-(Difluoromethyl)-2-fluoro[1,1′-biphenyl]-4-yl]-3,6-dihydro-2H-1,3,4-oxadiazin-2-one,
5-(2,4′-Difluoro-2′-methyl[1,1′-biphenyl]-4-yl)-3,6-dihydro-2H-1,3,4-oxadiazin-2-one,
2′-fluoro-2-methyl-4′-(2-oxo-3,6-dihydro-2H-1,3,4-oxadiazin-5-yl)[1,1′-biphenyl]-4-carbonitrile,
5-[4-(2-Methylprop-1-en-1-yl)-3-(trifluoromethoxy)phenyl]-3,6-dihydro-2H-1,3,4-oxadiazin-2-one,
(6S)-5-[4-(2-aminopyridin-4-yl)-3-(trifluoromethyl)phenyl]-6-methyl-3,6-dihydro-2H-1,3,4-oxadiazin-2-one,
(6S)-6-methyl-5-[4-(pyridin-4-yl)-3-(trifluoromethyl)phenyl]-3,6-dihydro-2H-1,3,4-oxadiazin-2-one,
(6S)-6-methyl-5-[4-(6-methylpyridin-3-yl)-3-(trifluoromethyl)phenyl]-3,6-dihydro-2H-1,3,4-oxadiazin-2-one,
(6S)-5-[2′-fluoro-4′-methyl-2-(trifluoromethyl)[1,1′-biphenyl]-4-yl]-6-methyl-3,6-dihydro-2H-1,3,4-oxadiazin-2-one,
(6S)-6-methyl-5-[2′,4′,5′-trifluoro-2-(trifluoromethyl)[1,1′-biphenyl]-4-yl]-3,6-dihydro-2H-1,3,4-oxadiazin-2-one,
(6S)-6-methyl-5-[2′,3′,4′-trifluoro-2-(trifluoromethyl)[1,1′-biphenyl]-4-yl]-3,6-dihydro-2H-1,3,4-oxadiazin-2-one,
(6S)-5-[2′,5′-difluoro-2-(trifluoromethyl)[1,1′-biphenyl]-4-yl]-6-methyl-3,6-dihydro-2H-1,3,4-oxadiazin-2-one,
4′-[(6S)-6-methyl-2-oxo-3,6-dihydro-2H-1,3,4-oxadiazin-5-yl]-2′-(trifluoromethyl)[1,1′-biphenyl]-2-carbonitrile,
(6S)-5-[4-(1H-indol-5-yl)-3-(trifluoromethyl)phenyl]-6-methyl-3,6-dihydro-2H-1,3,4-oxadiazin-2-one,
(6S)-5-[4′-hydroxy-2-(trifluoromethyl)[1,1′-biphenyl]-4-yl]-6-methyl-3,6-dihydro-2H-1,3,4-oxadiazin-2-one,
(6S)-5-[3′-hydroxy-2-(trifluoromethyl)[1,1′-biphenyl]-4-yl]-6-methyl-3,6-dihydro-2H-1,3,4-oxadiazin-2-one,
(6S)-5-[3′-amino-2-(trifluoromethyl)[1,1′-biphenyl]-4-yl]-6-methyl-3,6-dihydro-2H-1,3,4-oxadiazin-2-one,
(6S)-5-[2′,4′-difluoro-2-(trifluoromethyl)[1,1′-biphenyl]-4-yl]-6-methyl-3,6-dihydro-2H-1,3,4-oxadiazin-2-one,
(6S)-5-[3′-fluoro-4′-methyl-2-(trifluoromethyl)[1,1′-biphenyl]-4-yl]-6-methyl-3,6-dihydro-2H-1,3,4-oxadiazin-2-one,
(6S)-5-[2′-fluoro-2-(trifluoromethyl)[1,1′-biphenyl]-4-yl]-6-methyl-3,6-dihydro-2H-1,3,4-oxadiazin-2-one,
(6S)-5-[2′-methoxy-2-(trifluoromethyl)[1,1′-biphenyl]-4-yl]-6-methyl-3,6-dihydro-2H-1,3,4-oxadiazin-2-one,
(6S)-5-[3′-fluoro-2-(trifluoromethyl)[1,1′-biphenyl]-4-yl]-6-methyl-3,6-dihydro-2H-1,3,4-oxadiazin-2-one,
(6S)-6-methyl-5-[4-(4-methylpyridin-3-yl)-3-(trifluoromethyl)phenyl]-3,6-dihydro-2H-1,3,4-oxadiazin-2-one,
(6S)-6-methyl-5-[4-(3-methylpyridin-4-yl)-3-(trifluoromethyl)phenyl]-3,6-dihydro-2H-1,3,4-oxadiazin-2-one,
(6S)-6-methyl-5-[4-(2-methylpyridin-4-yl)-3-(trifluoromethyl)phenyl]-3,6-dihydro-2H-1,3,4-oxadiazin-2-one,
(6S)-5-[4-(1H-indol-6-yl)-3-(trifluoromethyl)phenyl]-6-methyl-3,6-dihydro-2H-1,3,4-oxadiazin-2-one,
(6S)-5-[4-(6-methoxypyridin-3-yl)-3-(trifluoromethyl)phenyl]-6-methyl-3,6-dihydro-2H-1,3,4-oxadiazin-2-one,
(6S)-5-[4′-methoxy-2-(trifluoromethyl)[1,1′-biphenyl]-4-yl]-6-methyl-3,6-dihydro-2H-1,3,4-oxadiazin-2-one,
(6S)-6-methyl-5-[4′-methyl-2-(trifluoromethyl)[1,1′-biphenyl]-4-yl]-3,6-dihydro-2H-1,3,4-oxadiazin-2-one,
(6S)-5-{4-[1-(difluoromethyl)-1H-pyrazol-4-yl]-3-(trifluoromethyl)-phenyl}-6-methyl-3,6-dihydro-2H-1,3,4-oxadiazin-2-one,
5-{4-[1-(difluoromethyl)-1H-pyrazol-4-yl]-3-(trifluoromethyl)-phenyl}-6-methyl-3,6-dihydro-2H-1,3,4-oxadiazin-2-one,
5-[4′-fluoro-2-(trifluoromethyl)[1,1′-biphenyl]-4-yl]-6-methyl-3,6-dihydro-2H-1,3,4-oxadiazin-2-one,
5-{4-[1-(difluoromethyl)-1H-pyrazol-4-yl]-3-fluoro-5-(trifluoromethyl)phenyl}-3,6-dihydro-2H-1,3,4-oxadiazin-2-one,
5-{3-(difluoromethyl)-4-[1-(difluoromethyl)-1H-pyrazol-4-yl]phenyl}-3,6-dihydro-2H-1,3,4-oxadiazin-2-one,
(6S)-6-methyl-5-{4-[(morpholin-4-yl)methyl]-3-(trifluoromethyl)phenyl}-3,6-dihydro-2H-1,3,4-oxadiazin-2-one,
5-{4-[(morpholin-4-yl)methyl]-3-(trifluoromethyl)phenyl}-3,6-dihydro-2H-1,3,4-oxadiazin-2-one,
5-[2-(difluoromethyl)-4′-fluoro[1,1′-biphenyl]-4-yl]-3,6-dihydro-2H-1,3,4-oxadiazin-2-one,
5-[4′-chloro-2-(difluoromethyl)[1,1′-biphenyl]-4-yl]-3,6-dihydro-2H-1,3,4-oxadiazin-2-one,
5-[3-(difluoromethyl)-4-(6-methylpyridin-3-yl)phenyl]-3,6-dihydro-2H-1,3,4-oxadiazin-2-one,
5-[4-(cyclopent-1-en-1-yl)-3-(difluoromethyl)phenyl]-3,6-dihydro-2H-1,3,4-oxadiazin-2-one,
5-[3-(difluoromethyl)-4-(1H-pyrazol-4-yl)phenyl]-3,6-dihydro-2H-1,3,4-oxadiazin-2-one,
5-[4-(3-Hydroxy-3-methylazetidin-1-yl)-3-(trifluoromethyl)phenyl]-3,6-dihydro-2H-1,3,4-oxadiazin-2-one,
(rac)-5-[4-{[3,3,3-trifluoro-2-hydroxypropyl]amino}-3-(trifluoromethyl)phenyl]-3,6-dihydro-2H-1,3,4-oxadiazin-2-one,
5-{4-[(Oxan-4-yl)amino]-3-(trifluoromethyl)phenyl}-3,6-dihydro-2H-1,3,4-oxadiazin-2-one,
5-[4-{[(cis/trans)-3-hydroxycyclobutyl]amino}-3-(trifluoromethyl)phenyl]-3,6-dihydro-2H-1,3,4-oxadiazin-2-one,
5-{4-[(rac)-2,4-Dimethylazetidin-1-yl]-3-(trifluoromethyl)phenyl}-3,6-dihydro-2H-1,3,4-oxadiazin-2-one,
5-{4-[(cis or trans)-2,4-Dimethylazetidin-1-yl]-3-(trifluoromethyl)phenyl}-3,6-dihydro-2H-1,3,4-oxadiazin-2-one,
5-[4-{[3,3,3-Trifluoro-2(S)-hydroxypropyl]amino}-3-(trifluoromethyl)phenyl]-3,6-dihydro-2H-1,3,4-oxadiazin-2-one,
5-{4-[(2-Hydroxy-2-methylpropyl)amino]-3-(trifluoromethyl)phenyl}-3,6-dihydro-2H-1,3,4-oxadiazin-2-one,
5-[4-{[(trans)-4-Hydroxycyclohexyl]amino}-3-(trifluoromethyl)phenyl]-3,6-dihydro-2H-1,3,4-oxadiazin-2-one,
5-{4-[(Cyclopropylmethyl)amino]-3-(trifluoromethyl)phenyl}-3,6-dihydro-2H-1,3,4-oxadiazin-2-one,
5-[4-{[(3-Methyloxetan-3-yl)methyl]amino}-3-(trifluoromethyl)phenyl]-3,6-dihydro-2H-1,3,4-oxadiazin-2-one,
-{4-[(3-Methoxypropyl)amino]-3-(trifluoromethyl)phenyl}-3,6-dihydro-2H-1,3,4-oxadiazin-2-one,
5-[4-({[(rac)-Oxolan-2-yl]methyl}amino)-3-(trifluoromethyl)phenyl]-3,6-dihydro-2H-1,3,4-oxadiazin-2-one,
5-[4-{[2(R)-2-Hydroxypropyl]amino}-3-(trifluoromethyl)phenyl]-3,6-dihydro-2H-1,3,4-oxadiazin-2-one,
5-[4-{[(3R)-3-Hydroxybutyl]amino}-3-(trifluoromethyl)phenyl]-3,6-dihydro-2H-1,3,4-oxadiazin-2-one,
5-[4-{[(2S)-2-Hydroxypropyl]amino}-3-(trifluoromethyl)phenyl]-3,6-dihydro-2H-1,3,4-oxadiazin-2-one,
5-[4-{[(1-Hydroxycyclobutyl)methyl]amino}-3-(trifluoromethyl)phenyl]-3,6-dihydro-2H-1,3,4-oxadiazin-2-one,
5-{4-[(3-Methylbutyl)amino]-3-(trifluoromethyl)phenyl}-3,6-dihydro-2H-1,3,4-oxadiazin-2-one,
5-{4-[(2-Methylpropyl)amino]-3-(trifluoromethyl)phenyl}-3,6-dihydro-2H-1,3,4-oxadiazin-2-one,
5-{4-[(2-Methoxyethyl)amino]-3-(trifluoromethyl)phenyl}-3,6-dihydro-2H-1,3,4-oxadiazin-2-one,
5-{4-[Ethyl(methyl)amino]-3-(trifluoromethyl)phenyl}-3,6-dihydro-2H-1,3,4-oxadiazin-2-one,
5-[4-(tert-butylamino)-3-(trifluoromethyl)phenyl]-3,6-dihydro-2H-1,3,4-oxadiazin-2-one,
5-[4-({[(2R)-oxolan-2-yl]methyl}amino)-3-(trifluoromethyl)phenyl]-3,6-dihydro-2H-1,3,4-oxadiazin-2-one,
5-[4-{[(pyrazin-2-yl)methyl]amino}-3-(trifluoromethyl)phenyl]-3,6-dihydro-2H-1,3,4-oxadiazin-2-one,
5-[4-(4-hydroxypiperidin-1-yl)-3-(trifluoromethyl)phenyl]-3,6-dihydro-2H-1,3,4-oxadiazin-2-one,
5-[4-{[(2S)-1-hydroxybutan-2-yl]amino}-3-(trifluoromethyl)phenyl]-3,6-dihydro-2H-1,3,4-oxadiazin-2-one,
5-[4-(3-hydroxypiperidin-1-yl)-3-(trifluoromethyl)phenyl]-3,6-dihydro-2H-1,3,4-oxadiazin-2-one
(racemic mixture),
(rac)-1-[4-(2-oxo-3,6-dihydro-2H-1,3,4-oxadiazin-5-yl)-2-(trifluoromethyl)phenyl]piperidine-3-carboxamide,
5-{4-[(3-hydroxy-2,2-dimethylpropyl)amino]-3-(trifluoromethyl)phenyl}-3,6-dihydro-2H-1,3,4-oxadiazin-2-one,
5-[4-(4,4-difluoropiperidin-1-yl)-3-(trifluoromethyl)phenyl]-3,6-dihydro-2H-1,3,4-oxadiazin-2-one,
5-[4-{[(1R,2R,4R)-bicyclo[2.2.1]heptan-2-yl]amino}-3-(trifluoromethyl)phenyl]-3,6-dihydro-2H-1,3,4-oxadiazin-2-one,
5-{4-[(3S)-3-hydroxypyrrolidin-1-yl]-3-(trifluoromethyl)phenyl}-3,6-dihydro-2H-1,3,4-oxadiazin-2-one,
(rac)-5-{4-[(2-hydroxy-3-methoxypropyl)amino]-3-(trifluoromethyl)phenyl}-3,6-dihydro-2H-1,3,4-oxadiazin-2-one,
5-[4-{[(1-methyl-1H-pyrazol-5-yl)methyl]amino}-3-(trifluoromethyl)phenyl]-3,6-dihydro-2H-1,3,4-oxadiazin-2-one,
5-[4-{[(1H-pyrazol-3-yl)methyl]amino}-3-(trifluoromethyl)phenyl]-3,6-dihydro-2H-1,3,4-oxadiazin-2-one,
5-[4-{[2-(1H-pyrazol-1-yl)ethyl]amino}-3-(trifluoromethyl)phenyl]-3,6-dihydro-2H-1,3,4-oxadiazin-2-one,
1-[4-(2-oxo-3,6-dihydro-2H-1,3,4-oxadiazin-5-yl)-2-(trifluoromethyl)phenyl]piperidine-4-carbonitrile,
(rac)-5-{4-[(1-cyclopropylethyl)amino]-3-(trifluoromethyl)phenyl}-3,6-dihydro-2H-1,3,4-oxadiazin-2-one,
(rac)-5-{4-[(2-ethoxypropyl)amino]-3-(trifluoromethyl)phenyl}-3,6-dihydro-2H-1,3,4-oxadiazin-2-one
(rac)-5-{4-[(2-methoxypropyl)amino]-3-(trifluoromethyl)phenyl}-3,6-dihydro-2H-1,3,4-oxadiazin-2-one
5-[4-(3-ethoxyazetidin-1-yl)-3-(trifluoromethyl)phenyl]-3,6-dihydro-2H-1,3,4-oxadiazin-2-one,
5-[4-{[(pyrimidin-2-yl)methyl]amino}-3-(trifluoromethyl)phenyl]-3,6-dihydro-2H-1,3,4-oxadiazin-2-one,
5-[4-{[(oxolan-3-yl)methyl]amino}-3-(trifluoromethyl)phenyl]-3,6-dihydro-2H-1,3,4-oxadiazin-2-one (racemic mixture),
5-[4-{[(2S)-4-hydroxybutan-2-yl]amino}-3-(trifluoromethyl)phenyl]-3,6-dihydro-2H-1,3,4-oxadiazin-2-one,
(rac)-5-[4-{[(6-oxopiperidin-3-yl)methyl]amino}-3-(trifluoromethyl)phenyl]-3,6-dihydro-2H-1,3,4-oxadiazin-2-one,
(rac)-5-[4-{[(2,2-dimethylcyclopropyl)methyl]amino}-3-(trifluoromethyl)phenyl]-3,6-dihydro-2H-1,3,4-oxadiazin-2-one,
5-[4-({[1-(hydroxymethyl)cyclobutyl]methyl}amino)-3-(trifluoromethyl)phenyl]-3,6-dihydro-2H-1,3,4-oxadiazin-2-one,
5-{4-[(2S)-2-(hydroxymethyl)azetidin-1-yl]-3-(trifluoromethyl)phenyl}-3,6-dihydro-2H-1,3,4-oxadiazin-2-one,
3-methyl-1-[4-(2-oxo-3,6-dihydro-2H-1,3,4-oxadiazin-5-yl)-2-(trifluoromethyl)phenyl]azetidine-3-carbonitrile,
5-[4-(3-azabicyclo[3.1.0]hexan-3-yl)-3-(trifluoromethyl)phenyl]-3,6-dihydro-2H-1,3,4-oxadiazin-2-one,
5-[4-(4-ethyl-4-hydroxypiperidin-1-yl)-3-(trifluoromethyl)phenyl]-3,6-dihydro-2H-1,3,4-oxadiazin-2-one,
4-[4-(2-oxo-3,6-dihydro-2H-1,3,4-oxadiazin-5-yl)-2-(trifluoromethyl)anilino]butanenitrile,
6-[4-(2-oxo-3,6-dihydro-2H-1,3,4-oxadiazin-5-yl)-2-(trifluoro-methyl)phenyl]-2lambda⁶-thia-6-azaspiro[3.3]heptane-2,2-dione,
N²-[4-(2-oxo-3,6-dihydro-2H-1,3,4-oxadiazin-5-yl)-2-(trifluoromethyl)phenyl]glycinamide,
5-{4-[(3R)-3-hydroxypyrrolidin-1-yl]-3-(trifluoromethyl)phenyl}-3,6-dihydro-2H-1,3,4-oxadiazin-2-one,
5-{4-[(2-methoxy-2-methylpropyl)amino]-3-(trifluoromethyl)phenyl}-3,6-dihydro-2H-1,3,4-oxadiazin-2-one
5-[4-({[(2S)-oxolan-2-yl]methyl}amino)-3-(trifluoromethyl)phenyl]-3,6-dihydro-2H-1,3,4-oxadiazin-2-one,
5-{4-[(2-ethoxyethyl)amino]-3-(trifluoromethyl)phenyl}-3,6-dihydro-2H-1,3,4-oxadiazin-2-one,
5-[4-{[(1S,2R)-2-hydroxycyclopentyl]amino}-3-(trifluoromethyl)phenyl]-3,6-dihydro-2H-1,3,4-oxadiazin-2-one,
5-{4-[(oxetan-3-yl)amino]-3-(trifluoromethyl)phenyl}-3,6-dihydro-2H-1,3,4-oxadiazin-2-on,
5-{3-(difluoromethyl)-4-[1-(propan-2-yl)-1H-pyrazol-4-yl]phenyl}-3,6-dihydro-2H-1,3,4-oxadiazin-2-one,
5-[3-fluoro-4-(morpholin-4-yl)-5-(trifluoromethyl)phenyl]-3,6-dihydro-2H-1,3,4-oxadiazin-2-one,
(6S)-6-methyl-5-{3-(trifluoromethyl)-4-[3-(trifluoromethyl)-1H-pyrazol-1-yl]phenyl}-3,6-dihydro-2H-1,3,4-oxadiazin-2-one,
(6S)-6-methyl-5-{3-(trifluoromethyl)-4-[4-(trifluoromethyl)-1H-imidazol-1-yl]phenyl}-3,6-dihydro-2H-1,3,4-oxadiazin-2-one,
5-[4-(3-Methoxypropyl)-3-(trifluoromethyl)phenyl]-3,6-dihydro-2H-1,3,4-oxadiazin-2-one,
5-[4-(2-methylpropyl)-3-(trifluoromethyl)phenyl]-3,6-dihydro-2H-1,3,4-oxadiazin-2-one,
5-[4-(3,3-dimethylbutyl)-3-(trifluoromethyl)phenyl]-3,6-dihydro-2H-1,3,4-oxadiazin-2-one,
5-[4-(Propan-2-yl)-3-(trifluoromethyl)phenyl]-3,6-dihydro-2H-1,3,4-oxadiazin-2-one,
(rac)-5-{4-[oxan-3-yl]-3-(trifluoromethyl)phenyl}-3,6-dihydro-2H-1,3,4-oxadiazin-2-one,
(trans)-5-{4-[4-hydroxycyclohexyl]-3-(trifluoromethyl)phenyl}-3,6-dihydro-2H-1,3,4-oxadiazin-2-one (trans isomer),
(cis)-5-{4-[4-hydroxycyclohexyl]-3-(trifluoromethyl)phenyl}-3,6-dihydro-2H-1,3,4-oxadiazin-2-one,
5-{4-[(2-Aminoethyl)amino]-3-(trifluoromethyl)phenyl}-3,6-dihydro-2H-1,3,4-oxadiazin-2-one-salt with hydrochloric acid,
5-{4-[1-amino-3-azabicyclo[3.1.0]hexan-3-yl]-3-(trifluoromethyl)-phenyl}-3,6-dihydro-2H-1,3,4-oxadiazin-2-one-salt with hydrochloric acid,
5-[4-(methylamino)-3-(trifluoromethyl)phenyl]-3,6-dihydro-2H-1,3,4-oxadiazin-2-one,
(6S)-6-methyl-5-[4-(4-methylpiperazin-1-yl)-3-(trifluoromethyl)phenyl]-3,6-dihydro-2H-1,3,4-oxadiazin-2-one,
5-[4-(2-hydroxypropan-2-yl)-3-(trifluoromethyl)phenyl]-3,6-dihydro-2H-1,3,4-oxadiazin-2-one,
(6S)-5-[4-(3,3-difluoroazetidin-1-yl)-3-(trifluoromethyl)phenyl]-6-methyl-3,6-dihydro-2H-1,3,4-oxadiazin-2-one and
(6S)-5-[4-(3-hydroxy-3-methylazetidin-1-yl)-3-(trifluoromethyl)-phenyl]-6-methyl-3,6-dihydro-2H-1,3,4-oxadiazin-2-one
   or a stereoisomer, a tautomer, a hydrate, a solvate, or a salt thereof, or a mixture of or the compounds mentioned above for use in the treatment of sarcoma.

In accordance with a further embodiment of the first aspect, the present invention provides the method of treatment of sarcoma comprising administering to a patient in need thereof the compound of general formula (I), supra, wherein the compound is selected from the group:
5-[4-(4,4-Difluoropiperidin-1-yl)-3-fluorophenyl]-3,6-dihydro-2H-1,3,4-oxadiazin-2-one
5-[4′-Fluoro-2-(trifluoromethyl)biphenyl-4-yl]-3,6-dihydro-2H-1,3,4-oxadiazin-2-one,
5-[3-Fluoro-4-(morpholin-4-yl)phenyl]-3,6-dihydro-2H-1,3,4-oxadiazin-2-one,
5-(4′-Fluoro-2-methylbiphenyl-4-yl)-3,6-dihydro-2H-1,3,4-oxadiazin-2-one,
5-(3′,4′-Difluoro-2-methylbiphenyl-4-yl)-3,6-dihydro-2H-1,3,4-oxadiazin-2-one
5-[4′-(Difluoromethyl)-2-fluoro[1,1′-biphenyl]-4-yl]-3,6-dihydro-2H-1,3,4-oxadiazin-2-one
(6S)-5-[4′-Fluoro-2-(trifluoromethyl)biphenyl-4-yl]-6-methyl-3,6-dihydro-2H-1,3,4-oxadiazin-2-one,
5-[4-Methyl-3-(trifluoromethyl)phenyl]-3,6-dihydro-2H-1,3,4-oxadiazin-2-one,
5-{4-[1-(Difluoromethyl)-1H-pyrazol-4-yl]-3-(trifluoromethyl)phenyl}-3,6-dihydro-2H-1,3,4-oxadiazin-2-one,
(6S)-6-Methyl-5-{3-(trifluoromethyl)-4-[3-(trifluoromethyl)-1H-pyrazol-1-yl]phenyl}-3,6-dihydro-2H-1,3,4-oxadiazin-2-one,
5-[4-(3-Methoxypropyl)-3-(trifluoromethyl)phenyl]-3,6-dihydro-2H-1,3,4-oxadiazin-2-one,
5-[4-(3,3-Dimethylbutyl)-3-(trifluoromethyl)phenyl]-3,6-dihydro-2H-1,3,4-oxadiazin-2-one and
(6S)-6-Methyl-5-[4-(4-methylpiperazin-1-yl)-3-(trifluoromethyl)phenyl]-3,6-dihydro-2H-1,3,4-oxadiazin-2-one
   or a stereoisomer, a tautomer, a hydrate, a solvate, or a salt thereof, or a mixture of same or anyone of these compounds for use in the treatment of sarcoma.

In accordance with a further embodiment of the first aspect, the present invention provides the method of treatment of sarcoma comprising administering to a patient in need thereof the compound of general formula (I), supra, wherein the compound is selected from the group:
5-[4-(4,4-Difluoropiperidin-1-yl)-3-fluorophenyl]-3,6-dihydro-2H-1,3,4-oxadiazin-2-one
5-[4′-fluoro-2-(trifluoromethyl)biphenyl-4-yl]-3,6-dihydro-2H-1,3,4-oxadiazin-2-one,
5-[3-Fluoro-4-(morpholin-4-yl)phenyl]-3,6-dihydro-2H-1,3,4-oxadiazin-2-one
5-(4′-fluoro-2-methylbiphenyl-4-yl)-3,6-dihydro-2H-1,3,4-oxadiazin-2-one,
5-(3′,4′-Difluoro-2-methylbiphenyl-4-yl)-3,6-dihydro-2H-1,3,4-oxadiazin-2-one
5-[4′-(Difluoromethyl)-2-fluoro[1,1′-biphenyl]-4-yl]-3,6-dihydro-2H-1,3,4-oxadiazin-2-one
5-[4-Methyl-3-(trifluoromethyl)phenyl]-3,6-dihydro-2H-1,3,4-oxadiazin-2-one
5-{4-[1-(Difluoromethyl)-1H-pyrazol-4-yl]-3-(trifluoromethyl)phenyl}-3,6-dihydro-2H-1,3,4-oxadiazin-2-one
(6S)-6-Methyl-5-{3-(trifluoromethyl)-4-[3-(trifluoromethyl)-1H-pyrazol-1-yl]phenyl}-3,6-dihydro-2H-1,3,4-oxadiazin-2-one
5-[4-(3-Methoxypropyl)-3-(trifluoromethyl)phenyl]-3,6-dihydro-2H-1,3,4-oxadiazin-2-one
5-[4-(3,3-Dimethylbutyl)-3-(trifluoromethyl)phenyl]-3,6-dihydro-2H-1,3,4-oxadiazin-2-one
   or a stereoisomer, a tautomer, a hydrate, a solvate, or a salt thereof, or a mixture of same or anyone of these compounds for use in the treatment of sarcoma.

In accordance with a further embodiment of the first aspect, the present invention provides the method of treatment of sarcoma comprising administering to a patient in need thereof the compound of general formula (I) wherein the compound is selected from the group
5-[4′-fluoro-2-(trifluoromethyl)biphenyl-4-yl]-3,6-dihydro-2H-1,3,4-oxadiazin-2-one,
5-(4′-fluoro-2-methylbiphenyl-4-yl)-3,6-dihydro-2H-1,3,4-oxadiazin-2-one,
5-[4′-chloro-2-(trifluoromethyl)biphenyl-4-yl]-3,6-dihydro-2H-1,3,4-oxadiazin-2-one,
(6S)-5-{4-[1-(difluoromethyl)-1H-pyrazol-4-yl]-3-(trifluoromethyl)-phenyl}-6-methyl-3,6-dihydro-2H-1,3,4-oxadiazin-2-one,
5-[4-{[3,3,3-Trifluoro-2(S)-hydroxypropyl]amino}-3-(trifluoromethyl)phenyl]-3,6-dihydro-2H-1,3,4-oxadiazin-2-one,
5-{4-[(2-Hydroxy-2-methylpropyl)amino]-3-(trifluoromethyl)phenyl}-3,6-dihydro-2H-1,3,4-oxadiazin-2-one,
5-{4-[(2-Methoxyethyl)amino]-3-(trifluoromethyl)phenyl}-3,6-dihydro-2H-1,3,4-oxadiazin-2-one,
(6S)-5-[4′-fluoro-2-(trifluoromethyl)biphenyl-4-yl]-6-methyl-3,6-dihydro-2H-1,3,4-oxadiazin-2-one,
5-{4-[1-(difluoromethyl)-1H-pyrazol-4-yl]-3-(trifluoromethyl)phenyl}-3,6-dihydro-2H-1,3,4-oxadiazin-2-one,
(6S)-5-[4-(2-aminopyridin-4-yl)-3-(trifluoromethyl)phenyl]-6-methyl-3,6-dihydro-2H-1,3,4-oxadiazin-2-one and
(6S)-6-methyl-5-{3-(trifluoromethyl)-4-[3-(trifluoromethyl)-1H-pyrazol-1-yl]phenyl}-3,6-dihydro-2H-1,3,4-oxadiazin-2-one,
   or a stereoisomer, a tautomer, a hydrate, a solvate, or a salt thereof, or a mixture of same or anyone of these compounds for use in the treatment of sarcoma.

In accordance with a further embodiment of the first aspect, the present invention provides the method of treatment of sarcoma comprising administering to a patient in need thereof the compound of general formula (I) wherein the compound is selected from the group
5-[4′-fluoro-2-(trifluoromethyl)biphenyl-4-yl]-3,6-dihydro-2H-1,3,4-oxadiazin-2-one,
5-(4′-fluoro-2-methylbiphenyl-4-yl)-3,6-dihydro-2H-1,3,4-oxadiazin-2-one,
5-[4′-chloro-2-(trifluoromethyl)biphenyl-4-yl]-3,6-dihydro-2H-1,3,4-oxadiazin-2-one and
(6S)-5-[4′-fluoro-2-(trifluoromethyl)biphenyl-4-yl]-6-methyl-3,6-dihydro-2H-1,3,4-oxadiazin-2-one,
   or a stereoisomer, a tautomer, a hydrate, a solvate, or a salt thereof, or a mixture of same or anyone of these compounds for use in the treatment of sarcoma.

In accordance with a further embodiment of the first aspect, the present invention provides the method of treatment of sarcoma comprising administering to a patient in need thereof the compound of general formula (I) wherein the compound is selected from the group
(6S)-5-{4-[1-(difluoromethyl)-1H-pyrazol-4-yl]-3-(trifluoromethyl)-phenyl}-6-methyl-3,6-dihydro-2H-1,3,4-oxadiazin-2-one,
5-[4-{[3,3,3-Trifluoro-2(S)-hydroxypropyl]amino}-3-(trifluoromethyl)phenyl]-3,6-dihydro-2H-1,3,4-oxadiazin-2-one,
5-{4-[(2-Hydroxy-2-methylpropyl)amino]-3-(trifluoromethyl)phenyl}-3,6-dihydro-2H-1,3,4-oxadiazin-2-one,
5-{4-[(2-Methoxyethyl)amino]-3-(trifluoromethyl)phenyl}-3,6-dihydro-2H-1,3,4-oxadiazin-2-one,
5-{4-[1-(difluoromethyl)-1H-pyrazol-4-yl]-3-(trifluoromethyl)phenyl}-3,6-dihydro-2H-1,3,4-oxadiazin-2-one,
(6S)-5-[4-(2-aminopyridin-4-yl)-3-(trifluoromethyl)phenyl]-6-methyl-3,6-dihydro-2H-1,3,4-oxadiazin-2-one and
(6S)-6-methyl-5-{3-(trifluoromethyl)-4-[3-(trifluoromethyl)-1H-pyrazol-1-yl]phenyl}-3,6-dihydro-2H-1,3,4-oxadiazin-2-one,
   or a stereoisomer, a tautomer, a hydrate, a solvate, or a salt thereof, or a mixture of same or anyone of these compounds for use in the treatment of sarcoma.

In accordance with a further embodiment of the first aspect, the present invention provides the method of treatment of sarcoma comprising administering to a patient in need thereof the compound of general formula (I) wherein the compound is selected from the group
5-[4′-fluoro-2-(trifluoromethyl)biphenyl-4-yl]-3,6-dihydro-2H-1,3,4-oxadiazin-2-one,
5-(4′-fluoro-2-methylbiphenyl-4-yl)-3,6-dihydro-2H-1,3,4-oxadiazin-2-one,
5-[4′-chloro-2-(trifluoromethyl)biphenyl-4-yl]-3,6-dihydro-2H-1,3,4-oxadiazin-2-one,
(6S)-5-{4-[1-(difluoromethyl)-1H-pyrazol-4-yl]-3-(trifluoromethyl)-phenyl}-6-methyl-3,6-dihydro-2H-1,3,4-oxadiazin-2-one,
5-[4-{[3,3,3-Trifluoro-2(S)-hydroxypropyl]amino}-3-(trifluoromethyl)phenyl]-3,6-dihydro-2H-1,3,4-oxadiazin-2-one,
5-{4-[(2-Hydroxy-2-methylpropyl)amino]-3-(trifluoromethyl)phenyl}-3,6-dihydro-2H-1,3,4-oxadiazin-2-one and
5-{4-[(2-Methoxyethyl)amino]-3-(trifluoromethyl)phenyl}-3,6-dihydro-2H-1,3,4-oxadiazin-2-one,
   or a stereoisomer, a tautomer, a hydrate, a solvate, or a salt thereof, or a mixture of same or anyone of these compounds for use in the treatment of sarcoma.

In accordance with a further embodiment of the first aspect, the present invention provides the method of treatment of sarcoma comprising administering to a patient in need thereof the compound of general formula (I) wherein the compound is selected from the group
(6S)-5-[4′-fluoro-2-(trifluoromethyl)biphenyl-4-yl]-6-methyl-3,6-dihydro-2H-1,3,4-oxadiazin-2-one,
5-{4-[1-(difluoromethyl)-1H-pyrazol-4-yl]-3-(trifluoromethyl)phenyl}-3,6-dihydro-2H-1,3,4-oxadiazin-2-one,
(6S)-5-[4-(2-aminopyridin-4-yl)-3-(trifluoromethyl)phenyl]-6-methyl-3,6-dihydro-2H-1,3,4-oxadiazin-2-one and
(6S)-6-methyl-5-{3-(trifluoromethyl)-4-[3-(trifluoromethyl)-1H-pyrazol-1-yl]phenyl}-3,6-dihydro-2H-1,3,4-oxadiazin-2-one,
   or a stereoisomer, a tautomer, a hydrate, a solvate, or a salt thereof, or a mixture of same or anyone of these compounds for use in the treatment of sarcoma.

In accordance with a further embodiment of the first aspect, the present invention provides the method of treatment of sarcoma comprising administering to a patient in need thereof the compound of general formula (I) wherein the compound is selected from the group
(6S)-5-[4′-fluoro-2-(trifluoromethyl)biphenyl-4-yl]-6-methyl-3,6-dihydro-2H-1,3,4-oxadiazin-2-one,
5-{4-[1-(difluoromethyl)-1H-pyrazol-4-yl]-3-(trifluoromethyl)phenyl}-3,6-dihydro-2H-1,3,4-oxadiazin-2-one,
(6S)-6-methyl-5-{3-(trifluoromethyl)-4-[3-(trifluoromethyl)-1H-pyrazol-1-yl]phenyl}-3,6-dihydro-2H-1,3,4-oxadiazin-2-one,
   or a stereoisomer, a tautomer, a hydrate, a solvate, or a salt thereof, or a mixture of same or anyone of these compounds for use in the treatment of sarcoma.

In accordance with a further embodiment of the first aspect, the present invention provides the method of treatment of sarcoma comprising administering to a patient in need thereof the compound of general formula (I) wherein the compound is selected from the group
(6S)-5-[4′-fluoro-2-(trifluoromethyl)biphenyl-4-yl]-6-methyl-3,6-dihydro-2H-1,3,4-oxadiazin-2-one,
(6S)-6-methyl-5-{3-(trifluoromethyl)-4-[3-(trifluoromethyl)-1H-pyrazol-1-yl]phenyl}-3,6-dihydro-2H-1,3,4-oxadiazin-2-one,
Methyl-5-[4-(4-methylpiperazin-1-yl)-3-(trifluoromethyl)phenyl]-3,6-dihydro-2H-1,3,4-oxadiazin-2-one
   or a stereoisomer, a tautomer, a hydrate, a solvate, or a salt thereof, or a mixture of same or anyone of these compounds for use in the treatment of sarcoma.

In accordance with a further embodiment of the first aspect, the present invention provides the method of treatment of sarcoma comprising administering to a patient in need thereof the compound of general formula (I) wherein the compound is selected from the group
(6S)-5-[4′-fluoro-2-(trifluoromethyl)biphenyl-4-yl]-6-methyl-3,6-dihydro-2H-1,3,4-oxadiazin-2-one,
Methyl-5-[4-(4-methylpiperazin-1-yl)-3-(trifluoromethyl)phenyl]-3,6-dihydro-2H-1,3,4-oxadiazin-2-one
   or a stereoisomer, a tautomer, a hydrate, a solvate, or a salt thereof, or a mixture of same or anyone of these compounds for use in the treatment of sarcoma.

In accordance with a further embodiment of the first aspect, the present invention provides the method of treatment of sarcoma comprising administering to a patient in need thereof the compound of general formula (I) wherein the compound is
5-[4-(4,4-Difluoropiperidin-1-yl)-3-fluorophenyl]-3,6-dihydro-2H-1,3,4-oxadiazin-2-one
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
5-[4′-fluoro-2-(trifluoromethyl)biphenyl-4-yl]-3,6-dihydro-2H-1,3,4-oxadiazin-2-one,
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
5-[3-Fluoro-4-(morpholin-4-yl)phenyl]-3,6-dihydro-2H-1,3,4-oxadiazin-2-one
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
5-(4′-Fluoro-2-methylbiphenyl-4-yl)-3,6-dihydro-2H-1,3,4-oxadiazin-2-one
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
5-(3′,4′-Difluoro-2-methylbiphenyl-4-yl)-3,6-dihydro-2H-1,3,4-oxadiazin-2-one
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
5-[4′-(Difluoromethyl)-2-fluoro[1,1′-biphenyl]-4-yl]-3,6-dihydro-2H-1,3,4-oxadiazin-2-one
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
(6S)-5-[4′-fluoro-2-(trifluoromethyl)biphenyl-4-yl]-6-methyl-3,6-dihydro-2H-1,3,4-oxadiazin-2-one,
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
5-[4-Methyl-3-(trifluoromethyl)phenyl]-3,6-dihydro-2H-1,3,4-oxadiazin-2-one
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
5-{4-[1-(Difluoromethyl)-1H-pyrazol-4-yl]-3-(trifluoromethyl)phenyl}-3,6-dihydro-2H-1,3,4-oxadiazin-2-one
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
(6S)-6-Methyl-5-{3-(trifluoromethyl)-4-[3-(trifluoromethyl)-1H-pyrazol-1-yl]phenyl}-3,6-dihydro-2H-1,3,4-oxadiazin-2-one
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
5-[4-(3-Methoxypropyl)-3-(trifluoromethyl)phenyl]-3,6-dihydro-2H-1,3,4-oxadiazin-2-one
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
5-[4-(3,3-Dimethylbutyl)-3-(trifluoromethyl)phenyl]-3,6-dihydro-2H-1,3,4-oxadiazin-2-one
   or a stereoisomer, a tautomer, a hydrate, a solvate, or a salt thereof, or a mixture of same or the compound for use in the treatment of sarcoma.

In accordance with a further embodiment of the first aspect, the present invention provides the method of treatment of sarcoma comprising administering to a patient in need thereof the compound of general formula (I) wherein the compound is
(6S)-6-Methyl-5-[4-(4-methylpiperazin-1-yl)-3-(trifluoromethyl)phenyl]-3,6-dihydro-2H-1,3,4-oxadiazin-2-one,
   or a stereoisomer, a tautomer, a hydrate, a solvate, or a salt thereof, or a mixture of same or the compound for use in the treatment of sarcoma.

In another embodiment the invention provides for the compounds of formula (I) or any subgroup disclosed herein for the manufacture of a medicament for the treatment of sarcoma.

In a particular further embodiment of the first aspect, the present invention includes combinations of two or more of the above mentioned embodiments under the heading “further embodiments of the first aspect of the present invention” for use in the treatment of sarcoma.

The compounds of general formula (I) for use for the treatment of sarcoma can be prepared according to the disclosure of WO2019/025562, which is incorporated herein by reference in its entirety and particularly in reference to the syntheses described for Examples 1-330 therein.

Compounds of the present invention can be utilized to treat sarcomas. This method comprises administering to a mammal in need thereof, including a human, an amount of a compound of formula (I), or a pharmaceutically acceptable salt, isomer, polymorph, metabolite, hydrate, solvate or ester thereof; which is effective to treat sarcoma.

The present invention also provides methods of treating sarcoma comprising administering an effective amount of a compound of formula (I) to a patient in need thereof.

The present invention further comprises a method of treatment of sarcoma comprising the steps of
(a) analysing a tumor sample of a patient whether PDE3A or PDE3B and SLFN12 are produced,
(b) in case expression of PDE3A or PDE3B and SLFN12 is found treating said patient with a compound of formula (I) as disclosed herein.

The method of analysis can be conducted as described in the art, e.g. J. Biol. Chem. (2020) 295 (11), 3431-3446.

Cancers of the sarcoma type have been characterized in humans, but also exist in other mammals, and can be treated by administering pharmaceutical compositions of the compounds of formula (I). Sarcomas occur on various locations of the body starting from mesenchymal cells which are forming bones and starting in bone, bone marrow or cartilage or connective tissues such as e.g. blood vessels, cartilage, deep skin, fat, fibrous tissues, joints, ligaments, lymph vessels, muscles, nerves and tendons. For cancers of the sarcoma type there may exist more than 70 subtypes of sarcoma including angiosarcoma, chondrosarcoma, dermatofibrosarcoma protuberans, desmoplastic small round cell tumors, epithelioid sarcoma, Ewing sarcoma, fibrous histiocytoma, gastrointestinal stromal tumor, Kaposi's sarcoma, leiomyosarcoma, liposarcoma, malignant peripheral nerve sheath tumors, myxofibrosarcoma, osteosarcoma, rhabdomyosarcoma, soft tissue sarcoma, solitary fibrous tumor, synovial sarcoma and undifferentiated pleomorphic sarcoma, malignant fibrous histiocytoma, lymphosarcoma, osteosarcoma, rhabdomyosarcoma, soft tissue sarcoma, and synovial sarcoma.

The term “treating” or “treatment” as used in the present text is used conventionally, e.g., the management or care of a subject for the purpose of combating, alleviating, reducing, relieving, improving the condition of sarcoma and if most successful let disappear the sarcoma completely. If treatment or prophylaxis is mentioned, treatment is preferred.

In accordance with a further aspect, the present invention provides for compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of sarcoma, especially in the treatment of sarcoma and more specifically in the treatment of sarcoma of the osteosarcoma, synovial sarcoma, soft tissue sarcoma, sarcoma (represented by the cell line SA10199, representing a recurrent malignant solitary fibrous tumor, see experimental section) malignant fibrous histiocytoma.

In accordance with a further aspect, the present invention provides for compounds of general formula (I), as described supra, or stereoisomers, tautomers, N-oxides, hydrates, solvates, and salts thereof, particularly pharmaceutically acceptable salts thereof, or mixtures of same, for use in the treatment of sarcoma, including osteosarcoma, synovial sarcoma, soft tissue sarcoma, sarcoma (represented by the cell line SA10199, representing a recurrent malignant solitary fibrous tumor, see experimental section) or fibrous histiocytoma.

In accordance with a further aspect, the present invention includes the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of sarcoma, especially in the treatment of sarcoma and more specifically in the treatment of osteosarcoma, synovial sarcoma, soft tissue sarcoma, sarcoma (represented by the cell line SA10199, representing a recurrent malignant solitary fibrous tumor, see experimental section) malignant fibrous histiocytoma.

In accordance with a further aspect, the present invention includes the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of sarcoma, especially in the treatment of osteosarcoma, synovial sarcoma, soft tissue sarcoma, sarcoma (represented by the cell line SA10199, representing a recurrent malignant solitary fibrous tumor, see experimental section) malignant fibrous histiocytoma.

In accordance with a further aspect, the present invention includes the use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of sarcoma, especially for the treatment of osteosarcoma, synovial sarcoma, soft tissue sarcoma, sarcoma (represented by the cell line SA10199, representing a recurrent malignant solitary fibrous tumor, see experimental section) malignant fibrous histiocytoma.

In accordance with a further aspect, the present invention includes a method of treatment or prophylaxis of sarcoma, especially a method of treatment of sarcoma and more specifically a method of treatment of osteosarcoma, synovial sarcoma, soft tissue sarcoma, sarcoma (represented by the cell line SA10199, representing recurrent malignant solitary fibrous tumor, see experimental section) malignant fibrous histiocytoma using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention includes pharmaceutical compositions, in particular a medicament, comprising a compound of general formula (I), as described supra, or a stereoisomer, a tautomer, a hydrate, a solvate, a salt thereof, particularly a pharmaceutically acceptable salt, or a mixture of same, and one or more excipients), in particular one or more pharmaceutically acceptable excipient(s) for use in the treatment of sarcoma, especially in the treatment of sarcoma and more specifically in the treatment of osteosarcoma, synovial sarcoma, soft tissue sarcoma, sarcoma (represented by the cell line SA10199, representing a recurrent malignant solitary fibrous tumor, see experimental section) malignant fibrous histiocytoma. Conventional procedures for preparing such pharmaceutical compositions in appropriate dosage forms can be utilized.

The present invention furthermore includes pharmaceutical compositions, in particular medicaments, which comprise at least one compound of formula (I), conventionally together with one or more pharmaceutically suitable excipients, for their use for the treatment of sarcoma, especially in the treatment of sarcoma and more specifically in the treatment of osteosarcoma, synovial sarcoma, soft tissue sarcoma, sarcoma (represented by the cell line SA10199, representing a recurrent malignant solitary fibrous tumor, see experimental section) malignant fibrous histiocytoma.

In accordance with a further aspect, the present invention includes the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of sarcoma, especially in the treatment of sarcoma and more specifically in the treatment of sarcoma of the soft tissue, osteosarcoma, malignant fibrous histiocytoma, sarcoma represented by SA10199 or synovial sarcoma.

In accordance with a further aspect, the present invention includes the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of sarcoma, especially in the treatment of sarcoma and more specifically in the treatment of sarcoma of the soft tissue, osteosarcoma, sarcoma represented by SA10199 or synovial sarcoma.

In accordance with a further aspect, the present invention includes the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of sarcoma, especially in the treatment of sarcoma and more specifically in the treatment of sarcoma of the soft tissue, malignant fibrous histiocytoma, sarcoma represented by SA10199 or synovial sarcoma.

In accordance with a further aspect, the present invention includes the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of sarcoma, especially in the treatment of sarcoma and more specifically in the treatment of osteosarcoma.

Furthermore one embodiment of the first aspect of the invention is a method of treating osteosarcoma, synovial sarcoma, soft tissue sarcoma, sarcoma (represented by the model S10199) or fibrous histiocytoma comprising administering (6S)-5-[4′-fluoro-2-(trifluoromethyl)biphenyl-4-yl]-6-methyl-3,6-dihydro-2H-1,3,4-oxadiazin-2-one to a patient in need thereof.

It is possible for the compounds for use in the treatment of sarcoma to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds of formula (I) to be administered in suitable administration forms.

For oral administration, it is possible to formulate the compounds of formula (I) to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds of formula (I) in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds of formula (I) can be incorporated into the stated administration forms. This can be effected in a manner known per se by mixing with pharmaceutically suitable excipients.

Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to the use of a pharmaceutical composition which comprise at least one compound according to the invention, conventionally together with one or more pharmaceutically suitable excipient(s), for the treatment of sarcoma.

In accordance with another aspect, the present invention includes pharmaceutical combinations, in particular medicaments, comprising at least one compound of general formula (I) and at least one or more further active ingredients, in particular for the treatment and/or prophylaxis of sarcoma.

Particularly, the present invention includes a pharmaceutical combination, which comprises:
one or more first active ingredients, in particular compounds of general formula (I) as defined supra, and
one or more further active ingredients (e.g., anti-cancer drugs), for the treatment of sarcoma.

As further active ingredients for combination e.g. Bevazizumab, Paliperidone or Trabectidin as additional sarcoma agent may be used.

Alternatively other anti-cancer drugs may be combined, such as: 131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, alpharadin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, cemiplimab, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tislelizumab, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The term “combination” in the present invention is used as known to persons skilled in the art, it being possible for said combination to be a fixed combination, a non-fixed combination or a kit-of-parts.

A “fixed combination” is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of formula (I) can be administered as the sole pharmaceutical agent or in combination with one or more other pharmaceutically active ingredients where the combination causes no unacceptable adverse effects for the treatment of sarcoma. For example, the compounds of formula (I) can be combined with known anti-sarcoma agents or other anti-cancer drugs.

Anti-sarcoma agents may be selected from e.g. Bevazizumab, Paliperidone, Trabectidin Vincristine, Actinmycin D, Doxorubicin and Cyclophosphamide which should not be considered an exclusive list.

Based upon standard laboratory techniques known to evaluate compounds useful for the treatment of sarcoma, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 500 mg/kg body weight per day, particularly about 0.001 mg/kg to about 200 mg/kg body weight per day, and more particularly from about 0.01 mg/kg to about 50 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight. For oral administration the dosing schedule maybe once or two time or three times daily and a dose range as referred to above for general dosing is possible.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound formula (I) or a pharmaceutically acceptable salt or ester or composition thereof can be ascertained by those skilled in the art using conventional treatment tests.

### Experimental Section

Abbreviations used have their meanings customary per se to the skilled person.

The compounds of general formula (I) for use for the treatment of sarcoma can be prepared according to the disclosure of WO2019/025562. The example numbers referred to in the experimental section herein refer to the example numbers in this publication.

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis or other methods for its production is not described in the experimental part, are either commercially available, or are known or may be formed from known ingredients by known methods by a person skilled in the art.

### Experimental Section—Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

The in vitro activity of the compounds can be demonstrated in the following assay:

### Assay 1

### Cell Proliferation Measurement In Vitro in the Human Sarcoma Cell Line G-292 Clone A141B1

The antiproliferative activity of the compounds of the general formula (I) was examined in vitro in the human sarcoma cell line G-292 clone A141B1 (from ATCC). For this purpose, the appropriate number of cells (1000) were plated in 384-well plates with appropriate growth medium McCoy's 5a Medium Modified, FCS 10% final (Biochrom; #S 0415); and incubated at 37° C. overnight. After 24 h, cells on one plate (0 h plate) were treated with 30 μl/cavity of CTG solution (Promega Cell Titer Glo (catalogue #G755B and G756B)) and incubated at room temperature for 10 min, and luminescence was measured by means of a VICTOR V (Perkin Elmer), in order to determine cell viability on commencement of treatment. The cells on the test plate were treated with a set of compounds of the general formula (I) as indicated below and incubated at 37° C. for 72 h. The compounds were added to the cells by means of an HP D300 digital dispenser in a 10-step 2.5-fold dilution series generally starting at a maximum final drug concentration of 100 nM. As control, the cells were treated with vehicle (DMSO at 0.3% final concentration). After 72 h, the cells were treated with 30 μl/cavity of CTG solution (Promega Cell Titer Glo (catalogue #G755B and G756B)) and incubated at room temperature for 10 min, and luminescence was measured by means of a VICTOR V (Perkin Elmer), in order to determine cell viability at the end of treatment. The percentage effect on cell growth and the IC50 derived therefrom were determined for each test substance using the values from the 0 h plate (=maximum inhibition) and the DMSO control (=minimum inhibition). The IC50 values were calculated using a 4-parameter fit.

In summary, all examples shown in Table 1 demonstrated anti-proliferative activity in the human sarcoma cell line G-292 clone A141B1 with IC50s ranging from 604-13 nM.

TABLE 1
Anti-proliferation IC₅₀ values of several examples in vitro in the human
osteosarcoma cell line G-292 clone A141B1

| Example | | |
|---|---|---|
| of | | |
| WO2019/ | | Target |
| 025562 | Compound | IC₅₀ [M] |
| 1 | 5-[4-(4,4-Difluoropiperidin-1-yl)-3-fluorophenyl]- | 2.60E−08 |
| | 3,6-dihydro-2H-1,3,4-oxadiazin-2-one | |
| 3 | 5-[4'-Fluoro-2-(trifluoromethyl)biphenyl-4-yl]-3,6- | 3.82E−08 |
| | dihydro-2H-1,3,4-oxadiazin-2-one | |
| 4 | 5-[3-Fluoro-4-(morpholin-4-yl)phenyl]-3,6- | 1.03E−07 |
| | dihydro-2H-1,3,4-oxadiazin-2-one | |
| 7 | 5-(4'-Fluoro-2-methylbiphenyl-4-yl)-3,6-dihydro- | 4.96E−08 |
| | 2H-1,3,4-oxadiazin-2-one | |
| 8 | 5-(3',4'-Difluoro-2-methylbiphenyl-4-yl)-3,6- | 9.68E−08 |
| | dihydro-2H-1,3,4-oxadiazin-2-one | |
| 92 | 5-[4'-(Difluoromethyl)-2-fluoro[1,1'-biphenyl]- | 3.51E−08 |
| | 4-yl]-3,6-dihydro-2H-1,3,4-oxadiazin-2-one | |
| 135 | (6S)-5-[4'-Fluoro-2-(trifluoromethyl)biphenyl- | 1.76E−08 |
| | 4-yl]-6-methyl-3,6-dihydro-2H-1,3,4-oxadiazin- | |
| | 2-one | |
| 139 | 5-[4-Methyl-3-(trifluoromethyl)phenyl]-3,6- | 1.93E−07 |
| | dihydro-2H-1,3,4-oxadiazin-2-one | |
| 146 | 5-{4-[1-(Difluoromethyl)-1H-pyrazol-4-yl]-3- | 1.25E−08 |
| | (trifluoromethyl)phenyl}-3,6-dihydro-2H-1,3,4- | |
| | oxadiazin-2-one | |
| 265 | (6S)-6-Methyl-5-{3-(trifluoromethyl)-4-[3- | 3.72E−08 |
| | (trifluoromethyl)-1H-pyrazol-1-yl]phenyl}-3,6- | |
| | dihydro-2H-1,3,4-oxadiazin-2-one | |
| 267 | 5-[4-(3-Methoxypropyl)-3-(trifluoromethyl)- | 1.68E−08 |
| | phenyl]-3,6-dihydro-2H-1,3,4-oxadiazin-2-one | |
| 269 | 5-[4-(3,3-Dimethylbutyl)-3-(trifluoromethyl)- | 8.57E−08 |
| | phenyl]-3,6-dihydro-2H-1,3,4-oxadiazin-2-one | |
| 277 | (6S)-6-Methyl-5-[4-(4-methylpiperazin-1-yl)-3- | 6.04E−07 |
| | (trifluoromethyl)phenyl]-3,6-dihydro-2H-1,3,4- | |
| | oxadiazin-2-one | |

### Assay 2

### In Vivo Xenotransplantation Models

The anti-tumor activities of (6S)-5-[4′-fluoro-2-(trifluoromethyl)biphenyl-4-yl]-6-methyl-3,6-dihydro-2H-1,3,4-oxadiazin-2-one was examined in murine xenotransplantation models of human cancer. For this purpose, mice were implanted subcutaneously with tumor pieces. At a mean tumor size of 20-40 mm² animals were randomized into treatment and control groups (at least n=10 animals/group) and treatment started with vehicle only or respective compound (formulation: 90% PEG400/10% Ethanol; application route: per os (“p.o.”), orally). The oral application volume was 10 ml/kg. In the case of twice daily treatments, the time interval between two applications per day was 6-7 h. The tumor size and the body weight were determined at least weekly. The tumor area was detected by means of an electronic caliper [length (mm)×width (mm)]. The experiment was ended when the study reached the pre-determined ethical endpoint based on German and European animal welfare regulations. In vivo anti-tumor efficacy is presented as T/C ratio at study end (Treatment/Control; mean tumor weight of treatment group/mean tumor weight of control group) in Table 2. A compound having a T/C below 0.5 is defined as active (i.e., effective). Statistical analysis was assessed using SigmaStat software. A one-way analysis of variance was performed and differences to the control were compared by a pair-wise comparison procedure (Dunn's method).

(6S)-5-[4′-fluoro-2-(trifluoromethyl)biphenyl-4-yl]-6-methyl-3,6-dihydro-2H-1,3,4-oxadiazin-2-one was effective in reduction of tumor volume in the sarcoma models used and specified in Table 2.

TABLE 2
Anti-tumor activity of Compound of (6S)-5-[4'-fluoro-2-
(trifluoromethyl)biphenyl-4-yl]-6-methyl-3,6-dihydro-2H-1,3,4-
oxadiazin-2-one in different human cancer xenograft models in mice

| Patient-derived | | | |
|---|---|---|---|
| xenograft Model | Tumor pieces isolated | Dose and | |
| (CrownBio) | from patient with | schedule | T/C |
| SA3831 | Osteosarcoma | 10 mg/kg 2QD | 0.01 |
| | | p.o. | |
| SA4058 | Synovial Sarcoma | 10 mg/kg 2QD | 0.12 |
| | | p.o | |
| SA16044 | Soft Tissue Sarcoma | 10 mg/kg 2QD | 0.33 |
| | | p.o | |
| SA10199 | Sarcoma (a recurrent | 10 mg/kg 2QD | 0.34 |
| | malignant solitary fibrous | p.o | |
| | tumor, subtype | | |
| | unknown) | | |
| SA10245 | Fibrous histiocytoma | 10 mg/kg 2QD | 0.73 |
| | | p.o | |

Patient-derived xenograft models established by direct transplantation of fresh human tumors are considered to more closely resemble the heterogeneity of the actual human tumor compared to xenograft models established from cell lines that have been cultured in vitro for decades. The anti-tumor activity of (6S)-5-[4′-fluoro-2-(trifluoromethyl)biphenyl-4-yl]-6-methyl-3,6-dihydro-2H-1,3,4-oxadiazin-2-one was assessed in patient-derived xenograft models of sarcoma, a tumor type consisting of many subtypes and a high medical need due to absence of approved targeted therapies. The tumor models are originating from CrownBio.

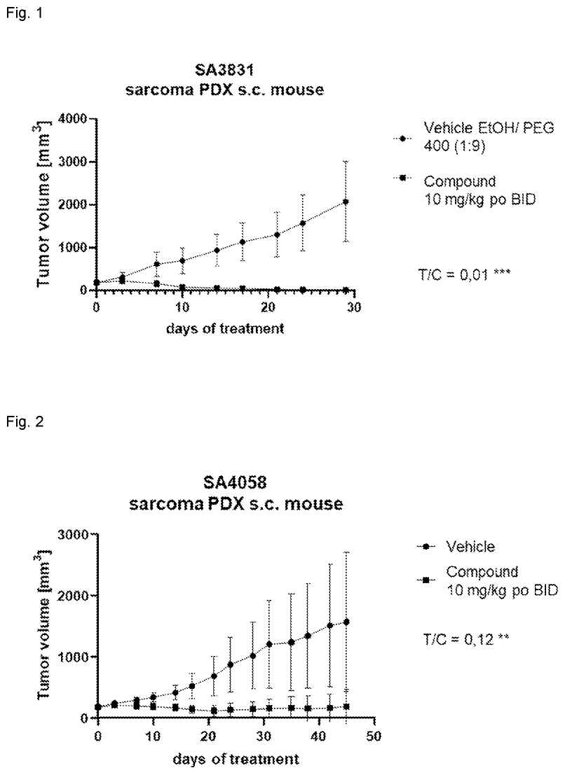

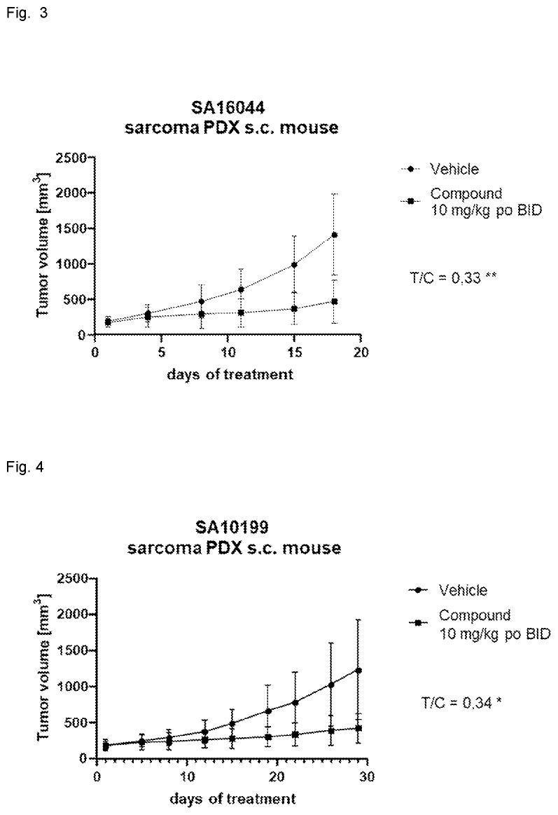

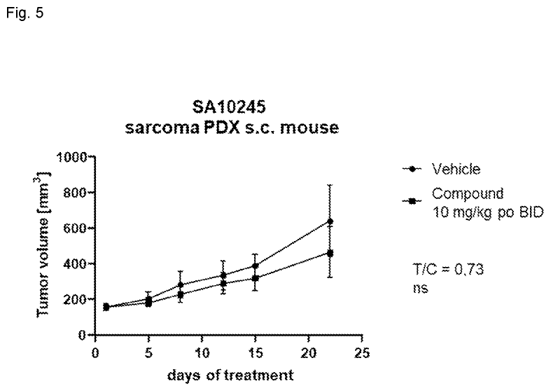

The patient-derived xenograft sarcoma models responded to treatment with (6S)-5-[4′-fluoro-2-(trifluoromethyl)biphenyl-4-yl]-6-methyl-3,6-dihydro-2H-1,3,4-oxadiazin-2-one with a reduced tumor growth compared to the vehicle control group. Model SA3831 was the most sensitive model, showing a strong tumor-growth inhibition reflected by a T/C ratio of 0.01 at the treatment end (FIG. 1). Model SA4058 was the second-most sensitive model, showing a strong tumor-growth inhibition reflected by a T/C ratio of 0.12 at the treatment end (FIG. 2). Models SA16044 and SA10199 also responded to treatment with (6S)-5-[4′-fluoro-2-(trifluoromethyl)biphenyl-4-yl]-6-methyl-3,6-dihydro-2H-1,3,4-oxadiazin-2-one showing a moderate tumor-growth inhibition reflected by a T/C ratio of 0.33 and 0.34, at the treatment end, respectively (FIGS. 3 & 4). The model SA10245 was the least sensitive model with a T/C ratio of only 0.73 (FIG. 5)

In summary, (6S)-5-[4′-fluoro-2-(trifluoromethyl)biphenyl-4-yl]-6-methyl-3,6-dihydro-2H-1,3,4-oxadiazin-2-one had significant anti-tumor activity in several patient-derived xenograft models of sarcoma.

Hence one embodiment of the first aspect of the invention is a method of treating sarcoma, more specifically osteosarcoma, synovial sarcoma, soft tissue sarcoma, sarcoma, represented by the cell line SA10199, or fibrous histiocytoma comprising administering (6S)-5-[4′-fluoro-2-(trifluoromethyl)biphenyl-4-yl]-6-methyl-3,6-dihydro-2H-1,3,4-oxadiazin-2-one to a patient in need thereof.

1. A method of inhibiting the growth or proliferation of a sarcoma in a subject, the method comprising administering to a subject having a sarcoma a compound of general formula (I): 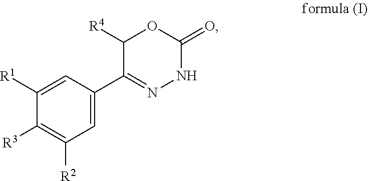
where
R¹ is selected from a hydrogen atom, a halogen atom, a cyano group, a C₁-C₃-alkyl group, a C₁-C₃-haloalkyl group, and a C₁-C₃-haloalkoxy group;
R² is selected from a hydrogen atom and a halogen atom;
R³ is selected from,
a C₁-C₆-alkyl group which is optionally substituted with one or two substituents and each substituent is independently selected from a hydroxy group, a C₁-C₄-alkoxy group and a 3- to 7-membered heterocycloalkyl group;
a C₂-C₆-alkenyl group which is optionally substituted with an C₁-C₄-alkoxy group;
a C₃-C₉-cycloalkyl group, which is optionally substituted with a hydroxy group;
a C₅-C₉-cycloalkenyl group, which is optionally substituted with a hydroxy group;
a 3- to 9-membered heterocycloalkyl group, comprising one, two or three heteroatoms which are independently selected from —O—, —S—, —S(O)—, S(O)₂, and —NR⁹—,
and said heterocycloalkyl group optionally further comprising a bridging group selected from —O—, —NR⁹—, —CH₂—, —CH₂—CH₂—, —O—CH₂—, —CH₂—O—, —NR⁹—CH₂—, and —CH₂—NR⁹—;
and said heterocycloalkyl group is optionally substituted with one, two or three substituents and each substituent is independently selected from
a halogen atom;
a oxo (═O) group;
a cyano group;
a hydroxy group;
a C₁-C₃-alkyl group which is optionally further substituted with a hydroxy group;
a C₁-C₃-haloalkyl group;
a C₁-C₃-alkoxy group;
a C₁-C₃-haloalkoxy group;
a C(O)NR⁵R⁶ group
and a NR⁵R⁶ group;
a 5- to 9-membered heterocycloalkyl group which is partially unsaturated and optionally substituted with one, two or three substituents and each substituent is independently selected from an oxo group (═O), a C₁-C₃-alkyl group, a —C(O)R⁵R⁶ group and a halogen atom;
an aryl group which is optionally substituted with one, two, three or four substituents and each substituent is independently selected from a halogen atom, a hydroxy group, a cyano group, a C₁-C₃-alkyl group, a C₁-C₃-haloalkyl group, a C₁-C₃-alkoxy group, a C₁-C₃-haloalkoxy group, and a NR⁵R⁶ group;
a mono- or bicyclic heteroaryl group which is optionally substituted with one, two or three substituents and each substituent is independently selected from a halogen atom, a C₁-C₃-alkyl group, a cyano group, a C₁-C₃-haloalkyl group, a C₁-C₃-hydroxalkyl group, a C₁-C₃-alkoxy group, a hydroxy group, and a NR⁵R⁶ group, with the proviso that said monocyclic heteroaryl group is not 4-pyridyl;
and a NR⁷R⁸ group;
R⁴ is selected from a hydrogen atom, and a C₁-C₃-alkyl group;
R⁵/R⁶ are independently selected from a hydrogen atom, a C₁-C₆-alkyl group, a —C₁-C₅-alkylene-O—C₁-C₅-alkyl group, a —C₁-C₅-alkylene-S—C₁-C₅-alkyl group, C₃-C₆-cycloalkyl group, and a C₃-C₅-heterocycloalkyl group;
R⁷/R⁸ are independently selected from a hydrogen atom, with the proviso that R⁷=R⁸=hydrogen is excluded,
a C₁-C₆-alkyl group,
which is optionally substituted with one, two, three or four substituents and said substituent is independently selected from
a halogen atom,
a cyano group,
a hydroxy group,
a C(O)NR⁵R⁶ group,
a NR⁵R⁶ group,
a C₁-C₃-alkoxy group,
a C₃-C₇-cycloalkyl group which is optionally substituted with one or two substituents and said substituents are independently selected from a C₁-C₃-alkyl group, a oxo (═O) group, a hydroxy group, and a C₁-C₃-hydroxyalkyl group;
a 3- to 7-membered heterocycloalkyl group which itself is optionally substituted with, a C₁-C₃-alkyl group or an oxo (═O) group;
a heteroaryl group, which itself is optionally substituted with a C₁-C₃-alkyl group;
a —C₁-C₅-alkylene-O—C₁-C₅-alkyl group;
a —C₁-C₅-alkylene-S—C₁-C₅-alkyl group;
a —C₁-C₅-alkylene-NR⁵—C₁-C₅-alkyl group;
a C₃-C₆-cycloalkyl group which is optionally substituted with a hydroxy group; and
a 3- to 6-membered heterocycloalkyl group, which is optionally substituted with one or two substituents, said substituent independently selected from C₁-C₃-alkyl group and a hydroxy group;
R⁹ is a hydrogen atom or a C₁-C₃-alkyl group or a bond;
or a stereoisomer, a tautomer, a hydrate, a solvate, or a salt thereof, or a mixture of same.

2. The method of treatment according to claim 1 in which:
R¹ is selected from a hydrogen atom, a halogen atom, a cyano group, a C₁-C₃-alkyl group, a C₁-C₃-haloalkyl group, and a C₁-C₃-haloalkoxy group;
R² is selected from a hydrogen atom and a halogen atom;
R³ is selected from
a C₁-C₆-alkyl group which is optionally substituted with a substituent which is selected from a hydroxy group, a C₁-C₄-alkoxy group, and a 3- to 7-membered heterocycloalkyl group;
a C₂-C₆-alkenyl group which is optionally substituted with an C₁-C₄-alkoxy group;
a C₃-C₇-cycloalkyl group, which is optionally substituted with a hydroxy group;
a C₅-C₇-cycloalkenyl group, which is optionally substituted with a hydroxy group;
a 3- to 7-membered-heterocycloalkyl group, comprising one, or two heteroatoms which are independently selected from —O—, S(O)₂, and —NR⁹—,
and said heterocycloalkyl group is optionally substituted with one, or two substituents and each substituent is independently selected from
a halogen atom;
a cyano group;
a hydroxy group;
a C₁-C₃-alkyl group which is optionally further substituted with a hydroxy group;
a C₁-C₃-alkoxy group;
a C(O)NR⁵R⁶ group and
a NR⁵R⁶ group;
a 5- to 7-membered-heterocycloalkyl group, comprising a heteroatom which is selected from —O—, —S— and —NR⁹—, which is partially unsaturated and optionally substituted with a substituent which is selected from a C₁-C₃-alkyl group and a halogen atom;
an aryl group which is optionally substituted with one, two, or three substituents and each substituent is independently selected from a halogen atom, a hydroxy group, a cyano group, a C₁-C₃-alkyl group, a C₁-C₃-haloalkyl group, a C₁-C₃-alkoxy group, and a NR⁵R⁶ group;
a mono- or bicyclic heteroaryl group which is optionally substituted with a substituent which is selected from a halogen atom, a C₁-C₃-alkyl group, a cyano group, a C₁-C₃-haloalkyl group, a C₁-C₃-alkoxy group, and a NR⁵R⁶ group, with the proviso that said monocyclic heteroaryl group is not a pyridin-4-yl group;
and a NR⁷R⁸ group;
R⁴ is selected from a hydrogen atom, and a C₁-C₃-alkyl group;
R⁵/R⁶ are independently selected from a hydrogen atom and a C₁-C₆-alkyl group;
R⁷/R⁸ are independently selected from
a hydrogen atom, with the proviso that R⁷=R⁸=hydrogen is excluded,
a C₁-C₆-alkyl group, which is optionally substituted with one, two, three or four substituents and said substituent is independently selected from
a halogen atom;
a cyano group;
a hydroxy group;
a C(O)NR⁵R⁶ group;
a NR⁵R⁶ group;
a C₁-C₃-alkoxy group;
a C₃-C₇-cycloalkyl group which is optionally further substituted with one or two substituents and said substituents are independently selected from a C₁-C₃-alkyl group, a oxo (═O) group, a hydroxy group, and a C₁-C₃-hydroxyalkyl group;
a 3- to 7-membered heterocycloalkyl group, comprising one, two or three heteroatoms which are independently selected from —O— and —NR⁹—, which is optionally further substituted with a C₁-C₃-alkyl group;
a heteroaryl group, which is optionally further substituted with a C₁-C₃-alkyl group;
a C₃-C₇-cycloalkyl group which is optionally substituted with a hydroxy group, or a C₁-C₃-alkyl group and
a 3- to 6-membered heterocycloalkyl group, which is optionally substituted with one or two substituents, said substituent independently selected from C₁-C₃-alkyl group and a hydroxy group;
R⁹ is a hydrogen atom or a C₁-C₃-alkyl group or a bond;
or a stereoisomer, a tautomer, a hydrate, a solvate, or a salt thereof, or a mixture of same.

3. The method of treatment according to claim 1 in which:
R¹ is selected from a hydrogen atom, a halogen atom, a C₁-C₃-alkyl group, a C₁-C₃-haloalkyl group, and a C₁-C₃-haloalkoxy group;
R² is selected from a hydrogen atom and a halogen atom;
R³ is selected from,
a C₁-C₆-alkyl group which is optionally substituted with a substituent which is selected from a hydroxy group, a C₁-C₄-alkoxy group, and a 3- to 7-membered heterocycloalkyl group;
a C₂-C₆-alkenyl group which is optionally substituted with an C₁-C₄-alkoxy group;
a C₃-C₇-cycloalkyl group, which is optionally substituted with a hydroxy group;
a C₅-C₇-cycloalkenyl group;
a 3- to 7-membered heterocycloalkyl group, comprising one, or two heteroatoms which are independently selected from —O—, and —NR⁹—,
and said heterocycloalkyl group is optionally further substituted with one, or two substituents and each substituent is independently selected from
a halogen atom;
a cyano group;
a hydroxy group;
a C₁-C₃-alkyl group which is optionally further substituted with a hydroxy group;
a C₁-C₃-alkoxy group and
a C(O)NR⁵R⁶ group;
a 5- to 7-membered-heterocycloalkyl group, comprising a heteroatom which is selected from —O—, and —NR⁹—, which is partially unsaturated and optionally substituted with a substituent which is selected from a C₁-C₃-alkyl group and a halogen atom,
an aryl group which is optionally substituted with one, two, or three substituents and each substituent is independently selected from a halogen atom, a hydroxy group, a cyano group, a C₁-C₃-alkyl group, a C₁-C₃-haloalkyl group, and a NR⁵R⁶ group;
a mono- or bicyclic heteroaryl group which is optionally substituted with a substituent which is selected from a halogen atom, a C₁-C₃-alkyl group a C₁-C₃-haloalkyl group, a C₁-C₃-alkoxy group, and a NR⁵R⁶ group, with the proviso that said monocyclic heteroaryl group is not a pyridin-4-yl group;
and a NR⁷R⁸ group;
R⁴ is selected from a hydrogen atom, and a C₁-C₃-alkyl group;
R⁵/R⁶ are independently selected from a hydrogen atom and a C₁-C₆-alkyl group;
R⁷/R⁸ are independently selected from
a hydrogen atom, with the proviso that R⁷=R⁸=hydrogen is excluded,
a C₁-C₆-alkyl group, which is optionally substituted with one, two, three or four substituents and said substituent is independently selected from
a halogen atom;
a cyano group;
a hydroxy group;
a NR⁵R⁶ group;
a C₁-C₃-alkoxy group;
a C₃-C₇-cycloalkyl group which is optionally further substituted with one or two substituents and said substituents are independently selected from a C₁-C₃-alkyl group, a oxo (═O) group, a hydroxy group, and a C₁-C₃-hydroxyalkyl group;
a 3- to 7-membered heterocycloalkyl group, comprising one, two or three heteroatoms which are independently selected from —O— and —NR⁹—, which is optionally further substituted with a C₁-C₃-alkyl group;
a heteroaryl group, which is optionally further substituted with a C₁-C₃-alkyl group;
a C₃-C₇-cycloalkyl group which is optionally substituted with a hydroxy group, or a C₁-C₃-alkyl group and
a 3- to 6-membered heterocycloalkyl group, which is optionally substituted with one or two substituents, said substituent independently selected from C₁-C₃-alkyl group and a hydroxy group;
R⁹ is a hydrogen atom or a C₁-C₃-alkyl group or a bond;
or a stereoisomer, a tautomer, a hydrate, a solvate, or a salt thereof, or a mixture of same.

4. The method for the treatment according to claim 1, in which:
R¹ is selected from a hydrogen atom, a halogen atom, a C₁-C₃-alkyl group, a C₁-C₃-haloalkyl group, and a C₁-C₃-haloalkoxy group;
R² is selected from a hydrogen atom and a halogen atom;
R³ is selected from,
a C₁-C₆-alkyl group which is optionally substituted with a substituent which is selected from a hydroxy group, a C₁-C₄-alkoxy group and a 3- to 7-membered heterocycloalkyl group;
a C₂-C₆-alkenyl group which is optionally substituted with an C₁-C₄-alkoxy group;
a C₄-C₆-cycloalkyl group, which is optionally substituted with a hydroxy group;
a C₅-C₇-cycloalkenyl group;
a 3- to 6-membered heterocycloalkyl group, comprising one, or two heteroatoms which are independently selected from —O—, and —NR⁹—,
and said heterocycloalkyl group is optionally further substituted with one, or two substituents and each substituent is independently selected from
a halogen atom;
a cyano group;
a hydroxy group;
a C₁-C₃-alkyl group which is optionally further substituted with a hydroxy group;
a 5- to 6-membered-heterocycloalkyl group, comprising a heteroatom which is selected from —O—, and —NR⁹—, which is partially unsaturated and optionally substituted with a substituent which is selected from a C₁-C₃-alkyl group and a halogen atom;
an aryl group which is optionally substituted with one, or two, substituents and each substituent is independently selected from a halogen atom, a hydroxy group, a cyano group, a C₁-C₃-alkyl group, a C₁-C₃-haloalkyl group, and a NR⁵R⁶ group;
a mono- or bicyclic heteroaryl group which is optionally substituted with a substituent which is selected from a halogen atom, a C₁-C₃-alkyl group a C₁-C₃-haloalkyl group, a C₁-C₃-alkoxy group, and a NR⁵R⁶ group, with the proviso that said monocyclic heteroaryl group is not a pyridin-4-yl group;
and a NR⁷R⁸ group;
R⁴ is selected from a hydrogen atom, and a C₁-C₃-alkyl group;
R⁵/R⁶ are independently selected from a hydrogen atom and a C₁-C₆-alkyl group;
R⁷/R⁸ are independently selected from
a hydrogen atom, with the proviso that R⁷=R⁸=hydrogen is excluded,
a C₁-C₆-alkyl group, which is optionally substituted with one, two, three or four substituents and said substituent is independently selected from
a halogen atom;
a hydroxy group;
a C₁-C₃-alkoxy group;
a C₃-C₆-cycloalkyl group which is optionally further substituted with one or two substituents and said substituents are independently selected from a C₁-C₃-alkyl group and a C₁-C₃-hydroxyalkyl group;
a 4- to 6-membered heterocycloalkyl group, comprising one, two or three heteroatoms which are independently selected from —O— and —NR⁹—, which is optionally further substituted with a C₁-C₃-alkyl group;
a heteroaryl group, which is optionally further substituted with a C₁-C₃-alkyl group;
a C₃-C₆-cycloalkyl group which is optionally substituted with a hydroxy group, or a C₁-C₃-alkyl group;
a 3- to 6-membered heterocycloalkyl group, which is optionally substituted with one or two substituents, said substituent independently selected from C₁-C₃-alkyl group and a hydroxy group, and
R⁹ is a hydrogen atom or a C₁-C₃-alkyl group or a bond;
or a stereoisomer, a tautomer, a hydrate, a solvate, or a salt thereof, or a mixture of same.

5. The method of treatment according to claim 1 in which:
R¹ is selected from a hydrogen atom, a halogen atom, a C₁-C₃-alkyl group, and a C₁-C₃-haloalkyl group;
R² is selected from a hydrogen atom and a halogen atom;
R³ is selected from,
a C₁-C₆-alkyl group which is optionally substituted with one or two substituents and each substituent is independently selected from a hydroxy group, a C₁-C₄-alkoxy group, and a 3- to 7-membered heterocycloalkyl group;
a C₂-C₆-alkenyl group which is optionally substituted with an C₁-C₃-alkoxy group;
a C₃-C₇-cycloalkyl group, which is optionally substituted with a hydroxy group;
a C₅-C₆-cycloalkenyl group, which is optionally substituted with a hydroxy group;
a 3- to 6-membered heterocycloalkyl group, comprising one, two or three heteroatoms which are independently selected from —O— and —NR⁹—,
and said heterocycloalkyl group is optionally substituted with one, two or three substituents and each substituent is independently selected from
a halogen atom;
a oxo (═O) group;
a cyano group;
a hydroxy group;
a C₁-C₃-alkyl group which is optionally further substituted with a hydroxy group;
a 5- to 7-membered heterocycloalkyl group which is partially unsaturated and optionally substituted with one or two substituents and each substituent is independently selected from a C₁-C₃-alkyl group and a halogen atom;
an aryl group which is optionally substituted with one, two, three or four substituents and each substituent is independently selected from a halogen atom, a hydroxy group, a cyano group, a C₁-C₃-alkyl group, a C₁-C₃-haloalkyl group, a C₁-C₃-alkoxy group, a C₁-C₃-haloalkoxy group, and a NR⁵R⁶ group;
a mono- or bicyclic heteroaryl group which is optionally substituted with one or two substituents and each substituent is independently selected from a halogen atom, a C₁-C₃-alkyl group, a cyano group, a C₁-C₃-haloalkyl group, a C₁-C₃-alkoxy group, a hydroxy group, and a NR⁵R⁶ group, with the proviso that said monocyclic heteroaryl group is not a pyridin-4-yl group;
and a NR⁷R⁸ group;
R⁴ is selected from a hydrogen atom, and a C₁-C₃-alkyl group;
R⁵/R⁶ are independently selected from a hydrogen atom and a C₁-C₆-alkyl group;
R⁷/R⁸ are independently selected from
a hydrogen atom, with the proviso that R⁷=R⁸=hydrogen is excluded,
a C₁-C₆-alkyl group,
which is optionally substituted with one, two, three or four substituents and said substituent is independently selected from
a halogen atom,
a cyano group,
a hydroxy group,
a C₁-C₃-alkoxy group,
a C₃-C₇-cycloalkyl group which is optionally substituted with one or two substituents and said substituents are independently selected from a C₁-C₃-alkyl group, a hydroxy group, and a C₁-C₃-hydroxyalkyl group;
a 3- to 7-membered heterocycloalkyl group which itself is optionally substituted with a C₁-C₃-alkyl group;
and a heteroaryl group, which is optionally further substituted with a C₁-C₃-alkyl group;
a C₃-C₆-cycloalkyl group which is optionally substituted with a hydroxy group, and
a 3- to 6-membered heterocycloalkyl group, which is optionally substituted with one or two substituents said substituent independently selected from C₁-C₃-alkyl group and a hydroxy group,
R⁹ is a hydrogen atom or a C₁-C₃-alkyl group or a bond;
or a stereoisomer, a tautomer, a hydrate, a solvate, or a salt thereof, or a mixture of same.

6. The method of treatment according to claim 1, in which:
R¹ is selected from a hydrogen atom, a halogen atom, a C₁-C₃-alkyl group, and a C₁-C₃-haloalkyl group;
R² is selected from a hydrogen atom and a halogen atom;
R³ is selected from,
a 3- to 6-membered heterocycloalkyl group, comprising one, or two heteroatoms which are independently selected from —O—, and —NR⁹—,
and said heterocycloalkyl group is optionally further substituted with one, or two substituents and each substituent is independently selected from
a halogen atom;
a hydroxy group;
and a C₁-C₃-alkyl group which is optionally further substituted with a hydroxy group;
an aryl group which is optionally substituted with one, or two, substituents and each substituent is independently selected from a halogen atom, a hydroxy group, and a C₁-C₃-haloalkyl group;
a mono- or bicyclic heteroaryl group which is optionally substituted with a substituent which is selected from a halogen atom, a C₁-C₃-alkyl group a C₁-C₃-haloalkyl group and a NR⁵R⁶ group, with the proviso that said monocyclic heteroaryl group is not a pyridin-4-yl group;
and a NR⁷R⁸ group;
R⁴ is selected from a hydrogen atom, and a C₁-C₃-alkyl group;
R⁵/R⁶ are independently selected from a hydrogen atom and a C₁-C₃-alkyl group;
R⁷/R⁸ are independently selected from
a hydrogen atom, with the proviso that R⁷=R⁸=hydrogen is excluded,
a C₁-C₆-alkyl group, which is optionally substituted with one or two, three or four substituents and said substituent is independently selected from
a halogen atom;
a hydroxy group;
a C₁-C₃-alkoxy group;
a C₃-C₅-cycloalkyl group which is optionally further substituted with one or two substituents and said substituents are independently selected from a C₁-C₃-alkyl group and a C₁-C₃-hydroxyalkyl group;
a 5- to 6-membered heterocycloalkyl group, comprising one, or two heteroatoms which are independently selected from —O— and —NR⁹—, which is optionally further substituted with a C₁-C₃-alkyl group,
a heteroaryl group, which is optionally further substituted with a C₁-C₃-alkyl group;
a C₃-C₆-cycloalkyl group which is optionally substituted with a hydroxy group, or a C₁-C₃-alkyl group;
a 4- to 5-membered heterocycloalkyl group, which is optionally substituted with one or two substituents, said substituent independently selected from C₁-C₃-alkyl group and a hydroxy group; and
R⁹ is a hydrogen atom or a C₁-C₃-alkyl group or a bond;
or a stereoisomer, a tautomer, a hydrate, a solvate, or a salt thereof, or a mixture of same.

7. The method of treatment according to claim 1, in which:
R¹ is selected from a C₁-C₃-alkyl group, a C₁-C₃-haloalkyl group and a halogen;
R² is a hydrogen atom;
R³ is selected from
a C₁-C₆-alkyl group, which is optionally substituted with a C₁-C₄-alkoxy group group
a 6-membered heterocycloalkyl group, comprising one, or two heteroatoms which are independently selected from —O— and —NR⁹—,
and said heterocycloalkyl group is optionally further substituted with one, or two substituents and each substituent is independently selected from
a halogen atom and a C₁-C₃-alkyl group,
a phenyl group, which is substituted with one or two substituents selected from a halogen atom or a C₁-C₃-haloalkyl group,
a monocyclic 5-membered heteroaryl group which is substituted with a substituent which is selected from a C₁-C₃-haloalkyl group;
R⁴ is selected from a hydrogen atom and a C₁-C₃-alkyl group;
R⁹ is a bond or C₁-C₃-alkyl group,
or a stereoisomer, a tautomer, a hydrate, a solvate, or a salt thereof, or a mixture of same or the compounds mentioned above for use in the treatment of sarcoma.

8. The method of treatment according to claim 1, in which:
R¹ is selected from CF₃ and a fluorine atom;
R² is a hydrogen atom;
R³ is selected from
an aryl group which is optionally substituted with a substituent which is selected from a halogen atom and a C₁-C₃-haloalkyl group,
a monocyclic heteroaryl group substituted with a substituent which is selected from C₁-C₃-haloalkyl group and NR⁵R⁶ group;
and a NR⁷R⁸ group;
R⁴ is selected from a hydrogen atom and a methyl group;
R₅/R⁶ are independently selected from a hydrogen atom and a methyl group;
R⁷/R⁸ are independently selected from
a hydrogen atom, with the proviso that R⁷=R⁸=hydrogen is excluded,
a C₁-C₃-alkyl group, which is optionally substituted with one, two or four substituents and said substituent is independently selected from
a halogen atom, a hydroxy group, and a methoxy group,
or a stereoisomer, a tautomer, a hydrate, a solvate, or a salt thereof, or a mixture of same.

9. The method of treatment according to claim 1, in which:
R¹ is selected from a CH₃ group, a CF₃ group and a fluorine atom;
R² is a hydrogen atom;
R³ is selected from
a halogen atom,
a C₁-C₆-alkyl group, which is optionally substituted with a methoxy group
a 6-membered heterocycloalkyl group, comprising one, or two heteroatoms which are independently selected from —O— and —NR⁹—,
and said heterocycloalkyl group is optionally further substituted with one, or two substituents and each substituent is independently selected from
a halogen atom and a C₁-C₃-alkyl group,
a phenyl group, which is substituted with one or two substituents selected from a halogen atom or a C₁-C₃-haloalkyl group,
a monocyclic 5-membered heteroaryl group which is substituted with a substituent which is selected from a C₁-C₃-haloalkyl group;
R⁴ is selected from a hydrogen atom and a methyl group;
R⁹ is a C₁-C₃-alkyl group,
or a stereoisomer, a tautomer, a hydrate, a solvate, or a salt thereof, or a mixture of same.

10. The method of treatment according to claim 1, wherein the compound of general formula (I), is selected from the group:
5-[4-(4,4-difluoropiperidin-1-yl)-3-fluorophenyl]-3,6-dihydro-2H-1,3,4-oxadiazin-2-one,
5-[4-chloro-3-(trifluoromethyl)phenyl]-3,6-dihydro-2H-1,3,4-oxadiazin-2-one,
5-[4′-fluoro-2-(trifluoromethyl)biphenyl-4-yl]-3,6-dihydro-2H-1,3,4-oxadiazin-2-one,
5-[3-fluoro-4-(morpholin-4-yl)phenyl]-3,6-dihydro-2H-1,3,4-oxadiazin-2-one,
5-[3-fluoro-4-(4-fluoro-4-methylpiperidin-1-yl)phenyl]-3,6-dihydro-2H-1,3,4-oxadiazin-2-one,
5-[3-fluoro-4-(4-fluoropiperidin-1-yl)phenyl]-3,6-dihydro-2H-1,3,4-oxadiazin-2-one,
5-(4′-fluoro-2-methylbiphenyl-4-yl)-3,6-dihydro-2H-1,3,4-oxadiazin-2-one,
5-(3′,4′-difluoro-2-methylbiphenyl-4-yl)-3,6-dihydro-2H-1,3,4-oxadiazin-2-one,
5-(4′-fluoro-2,2′-dimethylbiphenyl-4-yl)-3,6-dihydro-2H-1,3,4-oxadiazin-2-one,
5-[4-(3,6-dihydro-2H-pyran-4-yl)-3-methylphenyl]-3,6-dihydro-2H-1,3,4-oxadiazin-2-one,
5-[3-methyl-4-(1H-pyrazol-4-yl)phenyl]-3,6-dihydro-2H-1,3,4-oxadiazin-2-one,
5-[3-methyl-4-(6-oxo-1,6-dihydropyridin-3-yl)phenyl]-3,6-dihydro-2H-1,3,4-oxadiazin-2-one,
5-[3-methyl-4-(pyridin-3-yl)phenyl]-3,6-dihydro-2H-1,3,4-oxadiazin-2-one,
5-[3-methyl-4-(pyrimidin-5-yl)phenyl]-3,6-dihydro-2H-1,3,4-oxadiazin-2-one,
5-(3′-fluoro-2-methylbiphenyl-4-yl)-3,6-dihydro-2H-1,3,4-oxadiazin-2-one,
(rac)-6-methyl-5-(3,4,5-trifluorophenyl)-3,6-dihydro-2H-1,3,4-oxadiazin-2-one,
(rac)-5-[3,5-difluoro-4-(morpholin-4-yl)phenyl]-6-methyl-3,6-dihydro-2H-1,3,4-oxadiazin-2-one,
(rac)-5-{3,5-difluoro-4-[(2S)-2-methylmorpholin-4-yl]phenyl}-6-methyl-3,6-dihydro-2H-1,3,4-oxadiazin-2-one,
(rac)-5-(4-bromophenyl)-6-methyl-3,6-dihydro-2H-1,3,4-oxadiazin-2-one
(rac)-6-methyl-5-[4-(morpholin-4-yl)phenyl]-3,6-dihydro-2H-1,3,4-oxadiazin-2-one,
5-[3,5-difluoro-4-(morpholin-4-yl)phenyl]-3,6-dihydro-2H-1,3,4-oxadiazin-2-one,
2-(morpholin-4-yl)-5-(2-oxo-3,6-dihydro-2H-1,3,4-oxadiazin-5-yl)benzonitrile,
3-chloro-2-(morpholin-4-yl)-5-(2-oxo-3,6-dihydro-2H-1,3,4-oxadiazin-5-yl)benzonitrile,
5-{4-[2,6-dimethylmorpholin-4-yl]-3-fluorophenyl}-3,6-dihydro-2H-1,3,4-oxadiazin-2-one,
(6S)-5-(3-Fluoro-4-morpholinophenyl)-6-methyl-3,6-dihydro-2H-1,3,4-oxadiazin-2-one,
(6S)-5-(3,5-Difluoro-4-morpholinophenyl)-6-methyl-3,6-dihydro-2H-1,3,4-oxadiazin-2-one,
5-[4-(3,3-difluoropyrrolidin-1-yl)-3-fluorophenyl]-3,6-dihydro-2H-1,3,4-oxadiazin-2-one,
5-(2-methylbiphenyl-4-yl)-3,6-dihydro-2H-1,3,4-oxadiazin-2-one,
5-[3-methyl-4-(2-methylpyrimidin-5-yl)phenyl]-3,6-dihydro-2H-1,3,4-oxadiazin-2-one,
5-[3-methyl-4-(1-methyl-1H-pyrazol-5-yl)phenyl]-3,6-dihydro-2H-1,3,4-oxadiazin-2-one,
5-(2,4′-difluorobiphenyl-4-yl)-3,6-dihydro-2H-1,3,4-oxadiazin-2-one,
5-[4′-chloro-2-(trifluoromethyl)biphenyl-4-yl]-3,6-dihydro-2H-1,3,4-oxadiazin-2-one,
5-[4-(6-methylpyridin-3-yl)-3-(trifluoromethyl)phenyl]-3,6-dihydro-2H-1,3,4-oxadiazin-2-one,
5-[4-(pyridin-3-yl)-3-(trifluoromethyl)phenyl]-3,6-dihydro-2H-1,3,4-oxadiazin-2-one,
5-[4′-amino-2-(trifluoromethyl)biphenyl-4-yl]-3,6-dihydro-2H-1,3,4-oxadiazin-2-one,
5-[3′-hydroxy-4′-methyl-2-(trifluoromethyl)biphenyl-4-yl]-3,6-dihydro-2H-1,3,4-oxadiazin-2-one,
5-{3-(trifluoromethyl)-4-[6-(trifluoromethyl)pyridin-3-yl]phenyl}-3,6-dihydro-2H-1,3,4-oxadiazin-2-one,
5-[4′-fluoro-3′-hydroxy-2-(trifluoromethyl)biphenyl-4-yl]-3,6-dihydro-2H-1,3,4-oxadiazin-2-one,
5-[5′-amino-2′,4′-difluoro-2-(trifluoromethyl)biphenyl-4-yl]-3,6-dihydro-2H-1,3,4-oxadiazin-2-one,
5-[4′-amino-3′-fluoro-2-(trifluoromethyl)biphenyl-4-yl]-3,6-dihydro-2H-1,3,4-oxadiazin-2-one,
5-[4-(6-aminopyridin-3-yl)-3-(trifluoromethyl)phenyl]-3,6-dihydro-2H-1,3,4-oxadiazin-2-one,
5-[3′-amino-4′-chloro-2-(trifluoromethyl)biphenyl-4-yl]-3,6-dihydro-2H-1,3,4-oxadiazin-2-one,
5-[3′-amino-4′-methyl-2-(trifluoromethyl)biphenyl-4-yl]-3,6-dihydro-2H-1,3,4-oxadiazin-2-one,
5-[3′-amino-2′-fluoro-2-(trifluoromethyl)biphenyl-4-yl]-3,6-dihydro-2H-1,3,4-oxadiazin-2-one,
5-[4′-amino-2′-fluoro-2-(trifluoromethyl)biphenyl-4-yl]-3,6-dihydro-2H-1,3,4-oxadiazin-2-one,
5-[4-(1-methyl-1H-pyrazol-4-yl)-3-(trifluoromethyl)phenyl]-3,6-dihydro-2H-1,3,4-oxadiazin-2-one,
5-[4-(3-methyl-1H-pyrazol-4-yl)-3-(trifluoromethyl)phenyl]-3,6-dihydro-2H-1,3,4-oxadiazin-2-one,
5-[4-(1H-indazol-6-yl)-3-(trifluoromethyl)phenyl]-3,6-dihydro-2H-1,3,4-oxadiazin-2-one,
5-[4-(5-fluoro-6-methylpyridin-3-yl)-3-(trifluoromethyl)phenyl]-3,6-dihydro-2H-1,3,4-oxadiazin-2-one,
5-[4-(1,2-thiazol-4-yl)-3-(trifluoromethyl)phenyl]-3,6-dihydro-2H-1,3,4-oxadiazin-2-one,
1-methyl-5-[4-(2-oxo-3,6-dihydro-2H-1,3,4-oxadiazin-5-yl)-2-(trifluoromethyl)phenyl]-1H-pyrrole-2-carbonitrile,
5-[2,4′-bis(trifluoromethyl)biphenyl-4-yl]-3,6-dihydro-2H-1,3,4-oxadiazin-2-one,
5-[4-(1,3-dimethyl-1H-pyrazol-4-yl)-3-(trifluoromethyl)phenyl]-3,6-dihydro-2H-1,3,4-oxadiazin-2-one,
5-[4-(2-methoxy-6-methylpyridin-3-yl)-3-(trifluoromethyl)phenyl]-3,6-dihydro-2H-1,3,4-oxadiazin-2-one,
5-[4-(2-methyl-1,3-thiazol-5-yl)-3-(trifluoromethyl)phenyl]-3,6-dihydro-2H-1,3,4-oxadiazin-2-one,
5-[4′-(methylamino)-2-(trifluoromethyl)biphenyl-4-yl]-3,6-dihydro-2H-1,3,4-oxadiazin-2-one,
5-[3′-amino-4′-fluoro-2-(trifluoromethyl)biphenyl-4-yl]-3,6-dihydro-2H-1,3,4-oxadiazin-2-one,
5-[4-fluoro-3-(trifluoromethyl)phenyl]-3,6-dihydro-2H-1,3,4-oxadiazin-2-one,
5-[3′,4′,5′-trifluoro-2-(trifluoromethyl)biphenyl-4-yl]-3,6-dihydro-2H-1,3,4-oxadiazin-2-one,
5-[2′,5′-difluoro-2-(trifluoromethyl)biphenyl-4-yl]-3,6-dihydro-2H-1,3,4-oxadiazin-2-one,
5-[3′-amino-4′-fluoro-2-(trifluoromethoxy)biphenyl-4-yl]-3,6-dihydro-2H-1,3,4-oxadiazin-2-one,
5-[3′,4′-difluoro-2-(trifluoromethyl)biphenyl-4-yl]-3,6-dihydro-2H-1,3,4-oxadiazin-2-one,
5-[4-(1H-indol-6-yl)-3-(trifluoromethyl)phenyl]-3,6-dihydro-2H-1,3,4-oxadiazin-2-one,
5-[4-(2-methylprop-1-en-1-yl)-3-(trifluoromethyl)phenyl]-3,6-dihydro-2H-1,3,4-oxadiazin-2-one,
5-[2′,3′-difluoro-2-(trifluoromethyl)biphenyl-4-yl]-3,6-dihydro-2H-1,3,4-oxadiazin-2-one,
5-[4-(morpholin-4-yl)-3-(trifluoromethyl)phenyl]-3,6-dihydro-2H-1,3,4-oxadiazin-2-one,
5-[4-(butylamino)-3-(trifluoromethyl)phenyl]-3,6-dihydro-2H-1,3,4-oxadiazin-2-one,
5-[4-(ethylamino)-3-(trifluoromethyl)phenyl]-3,6-dihydro-2H-1,3,4-oxadiazin-2-one,
5-[4-(1-methyl-1H-pyrazol-4-yl)-3-(trifluoromethoxy)phenyl]-3,6-dihydro-2H-1,3,4-oxadiazin-2-one,
5-[4-(propylamino)-3-(trifluoromethyl)phenyl]-3,6-dihydro-2H-1,3,4-oxadiazin-2-one,
5-[4-(6-methylpyridin-3-yl)-3-(trifluoromethoxy)phenyl]-3,6-dihydro-2H-1,3,4-oxadiazin-2-one,
5-[4′-chloro-2-(trifluoromethoxy)biphenyl-4-yl]-3,6-dihydro-2H-1,3,4-oxadiazin-2-one,
5-[4-(azetidin-1-yl)-3-(trifluoromethyl)phenyl]-3,6-dihydro-2H-1,3,4-oxadiazin-2-one,
5-[4-(1-methyl-1H-benzimidazol-6-yl)-3-(trifluoromethyl)phenyl]-3,6-dihydro-2H-1,3,4-oxadiazin-2-one,
5-[4-(pentylamino)-3-(trifluoromethyl)phenyl]-3,6-dihydro-2H-1,3,4-oxadiazin-2-one,
5-[4-(1-methyl-1H-indazol-6-yl)-3-(trifluoromethyl)phenyl]-3,6-dihydro-2H-1,3,4-oxadiazin-2-one,
5-[4′-fluoro-2-(trifluoromethoxy)biphenyl-4-yl]-3,6-dihydro-2H-1,3,4-oxadiazin-2-one,
5-[3-fluoro-4-(6-fluoropyridin-3-yl)phenyl]-3,6-dihydro-2H-1,3,4-oxadiazin-2-one,
5-[3-fluoro-4-(3-methylpyridin-4-yl)phenyl]-3,6-dihydro-2H-1,3,4-oxadiazin-2-one,
5-[3-fluoro-4-(2-methylpyridin-4-yl)phenyl]-3,6-dihydro-2H-1,3,4-oxadiazin-2-one,
5-(4′-amino-2-fluorobiphenyl-4-yl)-3,6-dihydro-2H-1,3,4-oxadiazin-2-one,
5-(2-fluoro-2′-methylbiphenyl-4-yl)-3,6-dihydro-2H-1,3,4-oxadiazin-2-one,
5-(2′-chloro-2,4′-difluorobiphenyl-4-yl)-3,6-dihydro-2H-1,3,4-oxadiazin-2-one,
5-[4-(cyclopent-1-en-1-yl)-3-fluorophenyl]-3,6-dihydro-2H-1,3,4-oxadiazin-2-one,
5-(2′-ethyl-2-fluorobiphenyl-4-yl)-3,6-dihydro-2H-1,3,4-oxadiazin-2-one,
5-[3-fluoro-4-(6-methoxypyridin-3-yl)phenyl]-3,6-dihydro-2H-1,3,4-oxadiazin-2-one,
5-(2,4′-difluoro-3′-methylbiphenyl-4-yl)-3,6-dihydro-2H-1,3,4-oxadiazin-2-one,
5-(2-fluoro-3′-methylbiphenyl-4-yl)-3,6-dihydro-2H-1,3,4-oxadiazin-2-one,
5-(2-fluoro-4′-methylbiphenyl-4-yl)-3,6-dihydro-2H-1,3,4-oxadiazin-2-one,
5-(2-fluorobiphenyl-4-yl)-3,6-dihydro-2H-1,3,4-oxadiazin-2-one,
5-[4-(2-aminopyridin-4-yl)-3-fluorophenyl]-3,6-dihydro-2H-1,3,4-oxadiazin-2-one,
5-(3′-amino-2-fluorobiphenyl-4-yl)-3,6-dihydro-2H-1,3,4-oxadiazin-2-one,
5-[4′-(difluoromethyl)-2-fluorobiphenyl-4-yl]-3,6-dihydro-2H-1,3,4-oxadiazin-2-one,
5-[3-fluoro-4-(pyridin-4-yl)phenyl]-3,6-dihydro-2H-1,3,4-oxadiazin-2-one,
5-[3-fluoro-4-(2-methylpyrimidin-5-yl)phenyl]-3,6-dihydro-2H-1,3,4-oxadiazin-2-one,
5-[3-fluoro-4-(2-methoxypyridin-4-yl)phenyl]-3,6-dihydro-2H-1,3,4-oxadiazin-2-one,
5-[3-fluoro-4-(2-methylpyridin-3-yl)phenyl]-3,6-dihydro-2H-1,3,4-oxadiazin-2-one,
5-[3-fluoro-4-(6-methylpyridin-3-yl)phenyl]-3,6-dihydro-2H-1,3,4-oxadiazin-2-one,
5-(2,2′,4′,5′-tetrafluorobiphenyl-4-yl)-3,6-dihydro-2H-1,3,4-oxadiazin-2-one,
5-(2,2′,3′,4′-tetrafluorobiphenyl-4-yl)-3,6-dihydro-2H-1,3,4-oxadiazin-2-one,
5-(2,2′,5′-trifluorobiphenyl-4-yl)-3,6-dihydro-2H-1,3,4-oxadiazin-2-one,
2′-fluoro-4′-(2-oxo-3,6-dihydro-2H-1,3,4-oxadiazin-5-yl)biphenyl-4-carbonitrile,
5-(2′-amino-2-fluorobiphenyl-4-yl)-3,6-dihydro-2H-1,3,4-oxadiazin-2-one,
5-(3′-amino-2-fluoro-4′-methylbiphenyl-4-yl)-3,6-dihydro-2H-1,3,4-oxadiazin-2-one,
5-(2-fluoro-3′-hydroxybiphenyl-4-yl)-3,6-dihydro-2H-1,3,4-oxadiazin-2-one,
5-(2-fluoro-4′-hydroxybiphenyl-4-yl)-3,6-dihydro-2H-1,3,4-oxadiazin-2-one,
5-(2-fluoro-2′-hydroxybiphenyl-4-yl)-3,6-dihydro-2H-1,3,4-oxadiazin-2-one,
5-(2,3′,4′-trifluorobiphenyl-4-yl)-3,6-dihydro-2H-1,3,4-oxadiazin-2-one,
5-[3-fluoro-4-(pyridin-3-yl)phenyl]-3,6-dihydro-2H-1,3,4-oxadiazin-2-one,
5-(2,2′,3′-trifluorobiphenyl-4-yl)-3,6-dihydro-2H-1,3,4-oxadiazin-2-one,
5-(2,3′,5′-trifluorobiphenyl-4-yl)-3,6-dihydro-2H-1,3,4-oxadiazin-2-one,
5-(2,2′,4′-trifluorobiphenyl-4-yl)-3,6-dihydro-2H-1,3,4-oxadiazin-2-one,
5-(2-fluoro-2′,4′-dimethylbiphenyl-4-yl)-3,6-dihydro-2H-1,3,4-oxadiazin-2-one,
5-(2,3′-difluoro-4′-methylbiphenyl-4-yl)-3,6-dihydro-2H-1,3,4-oxadiazin-2-one,
5-(2,2′-difluorobiphenyl-4-yl)-3,6-dihydro-2H-1,3,4-oxadiazin-2-one,
5-(2,2′,6′-trifluorobiphenyl-4-yl)-3,6-dihydro-2H-1,3,4-oxadiazin-2-one,
5-(2-fluoro-2′-methoxybiphenyl-4-yl)-3,6-dihydro-2H-1,3,4-oxadiazin-2-one,
5-(2,3′-difluorobiphenyl-4-yl)-3,6-dihydro-2H-1,3,4-oxadiazin-2-one,
5-[3-fluoro-4-(4-methylpyridin-3-yl)phenyl]-3,6-dihydro-2H-1,3,4-oxadiazin-2-one,
(rac)-5-(3-Fluoro-4-morpholinophenyl)-6-methyl-3,6-dihydro-2H-1,3,4-oxadiazin-2-one,
(6S)-6-methyl-5-[4-(morpholin-4-yl)-3-(trifluoromethyl)phenyl]-3,6-dihydro-2H-1,3,4-oxadiazin-2-one,
(6S)-5-(-[(3-chloro-4-(morpholin-4-yl)-5-(trifluoromethyl)phenyl)-)]-6-methyl-3,6-dihydro-2H-1,3,4-oxadiazin-2-one,
6S)-5-(-[(4-chloro-3-(trifluoromethyl)phenyl)-)]-6-methyl-3,6-dihydro-2H-1,3,4-oxadiazin-2-one,
(6S)-5-(-[(4-fluoro-3-(trifluoromethyl)phenyl)-)]-6-methyl-3,6-dihydro-2H-1,3,4-oxadiazin-2-one,
5-[4-chloro-3-(trifluoromethoxy)phenyl]-3,6-dihydro-2H-1,3,4-oxadiazin-2-one,
5-(4-chloro-3-methylphenyl)-3,6-dihydro-2H-1,3,4-oxadiazin-2-one,
(rac)-6-methyl-5-(4-morpholino-3-(trifluoromethyl)phenyl)-3,6-dihydro-2H-1,3,4-oxadiazin-2-one,
5-{4-[1-methyl-3-(trifluoromethyl)-1H-pyrazol-4-yl]-3-(trifluoromethyl)phenyl}-3,6-dihydro-2H-1,3,4-oxadiazin-2-one,
5-[4-(3,5-dimethyl-1H-pyrazol-4-yl)-3-(trifluoromethyl)phenyl]-3,6-dihydro-2H-1,3,4-oxadiazin-2-one,
5-[4-(3,5-dimethyl-1,2-oxazol-4-yl)-3-(trifluoromethyl)phenyl]-3,6-dihydro-2H-1,3,4-oxadiazin-2-one,
5-{3-(trifluoromethyl)-4-[3-(trifluoromethyl)-1H-pyrazol-4-yl]phenyl}-3,6-dihydro-2H-1,3,4-oxadiazin-2-one,
5-[4-(1-ethyl-1H-pyrazol-4-yl)-3-(trifluoromethyl)phenyl]-3,6-dihydro-2H-1,3,4-oxadiazin-2-one,
5-{4-[cyclopentyl(methyl)amino]-3-(trifluoromethyl)phenyl}-3,6-dihydro-2H-1,3,4-oxadiazin-2-one,
5-[4-(1-methyl-1,2,3,6-tetrahydropyridin-4-yl)-3-(trifluoromethyl)phenyl]-3,6-dihydro-2H-1,3,4-oxadiazin-2-one,
5-{4-[butyl(methyl)amino]-3-(trifluoromethyl)phenyl}-3,6-dihydro-2H-1,3,4-oxadiazin-2-one,
(6S)-5-[4′-fluoro-2-(trifluoromethyl)biphenyl-4-yl]-6-methyl-3,6-dihydro-2H-1,3,4-oxadiazin-2-one,
5-[4-(cyclopentylamino)-3-(trifluoromethyl)phenyl]-3,6-dihydro-2H-1,3,4-oxadiazin-2-one,
5-[4-(cyclopentylamino)-3-(trifluoromethyl)phenyl]-3,6-dihydro-2H-1,3,4-oxadiazin-2-one,
5-[3′-fluoro-2-(trifluoromethyl)biphenyl-4-yl]-3,6-dihydro-2H-1,3,4-oxadiazin-2-one,
5-[4-methyl-3-(trifluoromethyl)phenyl]-3,6-dihydro-2H-1,3,4-oxadiazin-2-one,
5-{4-[3-methoxyprop-1-en-1-yl]-3-(trifluoromethyl)phenyl}-3,6-dihydro-2H-1,3,4-oxadiazin-2-one,
(rac)-5-[4′-hydroxy-2-(trifluoromethyl)-2′,3′,4′,5′-tetrahydro[1,1′-biphenyl]-4-yl]-3,6-dihydro-2H-1,3,4-oxadiazin-2-one,
5-[4-(5,6-dihydro-2H-pyran-3-yl)-3-(trifluoromethyl)phenyl]-3,6-dihydro-2H-1,3,4-oxadiazin-2-one,
5-[4-(imidazo[1,2-a]pyridin-6-yl)-3-(trifluoromethyl)phenyl]-3,6-dihydro-2H-1,3,4-oxadiazin-2-one,
5-{4-[3,3-dimethylbut-1-en-1-yl]-3-(trifluoromethyl)phenyl}-3,6-dihydro-2H-1,3,4-oxadiazin-2-one,
5-{3-(trifluoromethyl)-4-[5-(trifluoromethyl)thiophen-3-yl]phenyl}-3,6-dihydro-2H-1,3,4-oxadiazin-2-one,
5-{4-[1-(difluoromethyl)-1H-pyrazol-4-yl]-3-(trifluoromethyl)phenyl}-3,6-dihydro-2H-1,3,4-oxadiazin-2-one,
5-[4-(prop-1-en-2-yl)-3-(trifluoromethyl)phenyl]-3,6-dihydro-2H-1,3,4-oxadiazin-2-one,
5-[4-(1-benzothiophen-2-yl)-3-(trifluoromethyl)phenyl]-3,6-dihydro-2H-1,3,4-oxadiazin-2-one,
5-[4-(2,5-dihydrofuran-3-yl)-3-(trifluoromethyl)phenyl]-3,6-dihydro-2H-1,3,4-oxadiazin-2-one,
5-[4-(cyclopent-1-en-1-yl)-3-(trifluoromethyl)phenyl]-3,6-dihydro-2H-1,3,4-oxadiazin-2-one,
5-[4-(1-ethyl-1H-imidazol-4-yl)-3-(trifluoromethyl)phenyl]-3,6-dihydro-2H-1,3,4-oxadiazin-2-one,
3-methyl-5-[4-(2-oxo-3,6-dihydro-2H-1,3,4-oxadiazin-5-yl)-2-(trifluoromethyl)phenyl]thiophene-2-carbonitrile,
5-{4-[1-(propan-2-yl)-1H-pyrazol-4-yl]-3-(trifluoromethyl)phenyl}-3,6-dihydro-2H-1,3,4-oxadiazin-2-one,
(rac)-5-[4-(bicyclo[2.2.1]hept-2-en-2-yl)-3-(trifluoromethyl)phenyl]-3,6-dihydro-2H-1,3,4-oxadiazin-2-one,
5-[2′-fluoro-2-(trifluoromethyl)[1,1′-biphenyl]-4-yl]-3,6-dihydro-2H-1,3,4-oxadiazin-2-one,
5-{3-(Trifluoromethyl)-4-[5-(trifluoromethyl)thiophen-2-yl]phenyl}-3,6-dihydro-2H-1,3,4-oxadiazin-2-one,
5-[4-(5-methylpyridin-2-yl)-3-(trifluoromethyl)phenyl]-3,6-dihydro-2H-1,3,4-oxadiazin-2-one,
5-[4-(5-fluoropyridin-2-yl)-3-(trifluoromethyl)phenyl]-3,6-dihydro-2H-1,3,4-oxadiazin-2-one,
5-[4-(5-chloropyridin-2-yl)-3-(trifluoromethyl)phenyl]-3,6-dihydro-2H-1,3,4-oxadiazin-2-one,
5-[4-(pyridin-2-yl)-3-(trifluoromethyl)phenyl]-3,6-dihydro-2H-1,3,4-oxadiazin-2-one,
5-[2′-(Difluoromethyl)-2-fluoro[1,1′-biphenyl]-4-yl]-3,6-dihydro-2H-1,3,4-oxadiazin-2-one,
5-(2,4′-Difluoro-2′-methyl[1,1′-biphenyl]-4-yl)-3,6-dihydro-2H-1,3,4-oxadiazin-2-one,
2′-fluoro-2-methyl-4′-(2-oxo-3,6-dihydro-2H-1,3,4-oxadiazin-5-yl)[1,1′-biphenyl]-4-carbonitrile,
5-[4-(2-Methylprop-1-en-1-yl)-3-(trifluoromethoxy)phenyl]-3,6-dihydro-2H-1,3,4-oxadiazin-2-one,
(6S)-5-[4-(2-aminopyridin-4-yl)-3-(trifluoromethyl)phenyl]-6-methyl-3,6-dihydro-2H-1,3,4-oxadiazin-2-one,
(6S)-6-methyl-5-[4-(pyridin-4-yl)-3-(trifluoromethyl)phenyl]-3,6-dihydro-2H-1,3,4-oxadiazin-2-one,
(6S)-6-methyl-5-[4-(6-methylpyridin-3-yl)-3-(trifluoromethyl)phenyl]-3,6-dihydro-2H-1,3,4-oxadiazin-2-one,
(6S)-5-[2′-fluoro-4′-methyl-2-(trifluoromethyl)[1,1′-biphenyl]-4-yl]-6-methyl-3,6-dihydro-2H-1,3,4-oxadiazin-2-one,
(6S)-6-methyl-5-[2′,4′,5′-trifluoro-2-(trifluoromethyl)[1,1′-biphenyl]-4-yl]-3,6-dihydro-2H-1,3,4-oxadiazin-2-one,
(6S)-6-methyl-5-[2′,3′,4′-trifluoro-2-(trifluoromethyl)[1,1′-biphenyl]-4-yl]-3,6-dihydro-2H-1,3,4-oxadiazin-2-one,
(6S)-5-[2′,5′-difluoro-2-(trifluoromethyl)[1,1′-biphenyl]-4-yl]-6-methyl-3,6-dihydro-2H-1,3,4-oxadiazin-2-one,
4′-[(6S)-6-methyl-2-oxo-3,6-dihydro-2H-1,3,4-oxadiazin-5-yl]-2′-(trifluoromethyl)[1,1′-biphenyl]-2-carbonitrile,
(6S)-5-[4-(1H-indol-5-yl)-3-(trifluoromethyl)phenyl]-6-methyl-3,6-dihydro-2H-1,3,4-oxadiazin-2-one,
(6S)-5-[4′-hydroxy-2-(trifluoromethyl)[1,1′-biphenyl]-4-yl]-6-methyl-3,6-dihydro-2H-1,3,4-oxadiazin-2-one,
(6S)-5-[3′-hydroxy-2-(trifluoromethyl)[1,1′-biphenyl]-4-yl]-6-methyl-3,6-dihydro-2H-1,3,4-oxadiazin-2-one,
(6S)-5-[3′-amino-2-(trifluoromethyl)[1,1′-biphenyl]-4-yl]-6-methyl-3,6-dihydro-2H-1,3,4-oxadiazin-2-one,
(6S)-5-[2′,4′-difluoro-2-(trifluoromethyl)[1,1′-biphenyl]-4-yl]-6-methyl-3,6-dihydro-2H-1,3,4-oxadiazin-2-one,
(6S)-5-[3′-fluoro-4′-methyl-2-(trifluoromethyl)[1,1′-biphenyl]-4-yl]-6-methyl-3,6-dihydro-2H-1,3,4-oxadiazin-2-one,
(6S)-5-[2′-fluoro-2-(trifluoromethyl)[1,1′-biphenyl]-4-yl]-6-methyl-3,6-dihydro-2H-1,3,4-oxadiazin-2-one,
(6S)-5-[2′-methoxy-2-(trifluoromethyl)[1,1′-biphenyl]-4-yl]-6-methyl-3,6-dihydro-2H-1,3,4-oxadiazin-2-one,
(6S)-5-[3′-fluoro-2-(trifluoromethyl)[1,1′-biphenyl]-4-yl]-6-methyl-3,6-dihydro-2H-1,3,4-oxadiazin-2-one,
(6S)-6-methyl-5-[4-(4-methylpyridin-3-yl)-3-(trifluoromethyl)phenyl]-3,6-dihydro-2H-1,3,4-oxadiazin-2-one,
(6S)-6-methyl-5-[4-(3-methylpyridin-4-yl)-3-(trifluoromethyl)phenyl]-3,6-dihydro-2H-1,3,4-oxadiazin-2-one,
(6S)-6-methyl-5-[4-(2-methylpyridin-4-yl)-3-(trifluoromethyl)phenyl]-3,6-dihydro-2H-1,3,4-oxadiazin-2-one,
(6S)-5-[4-(1H-indol-6-yl)-3-(trifluoromethyl)phenyl]-6-methyl-3,6-dihydro-2H-1,3,4-oxadiazin-2-one,
(6S)-5-[4-(6-methoxypyridin-3-yl)-3-(trifluoromethyl)phenyl]-6-methyl-3,6-dihydro-2H-1,3,4-oxadiazin-2-one,
(6S)-5-[4′-methoxy-2-(trifluoromethyl)[1,1′-biphenyl]-4-yl]-6-methyl-3,6-dihydro-2H-1,3,4-oxadiazin-2-one,
(6S)-6-methyl-5-[4′-methyl-2-(trifluoromethyl)[1,1′-biphenyl]-4-yl]-3,6-dihydro-2H-1,3,4-oxadiazin-2-one,
(6S)-5-{4-[1-(difluoromethyl)-1H-pyrazol-4-yl]-3-(trifluoromethyl)-phenyl}-6-methyl-3,6-dihydro-2H-1,3,4-oxadiazin-2-one,
5-{4-[1-(difluoromethyl)-1H-pyrazol-4-yl]-3-(trifluoromethyl)-phenyl}-6-methyl-3,6-dihydro-2H-1,3,4-oxadiazin-2-one,
5-[4′-fluoro-2-(trifluoromethyl)[1,1′-biphenyl]-4-yl]-6-methyl-3,6-dihydro-2H-1,3,4-oxadiazin-2-one,
5-{4-[1-(difluoromethyl)-1H-pyrazol-4-yl]-3-fluoro-5-(trifluoromethyl)phenyl}-3,6-dihydro-2H-1,3,4-oxadiazin-2-one,
5-{3-(difluoromethyl)-4-[1-(difluoromethyl)-1H-pyrazol-4-yl]phenyl}-3,6-dihydro-2H-1,3,4-oxadiazin-2-one,
(6S)-6-methyl-5-{4-[(morpholin-4-yl)methyl]-3-(trifluoromethyl)phenyl}-3,6-dihydro-2H-1,3,4-oxadiazin-2-one,
5-{4-[(morpholin-4-yl)methyl]-3-(trifluoromethyl)phenyl}-3,6-dihydro-2H-1,3,4-oxadiazin-2-one,
5-[2-(difluoromethyl)-4′-fluoro[1,1′-biphenyl]-4-yl]-3,6-dihydro-2H-1,3,4-oxadiazin-2-one,
5-[4′-chloro-2-(difluoromethyl)[1,1′-biphenyl]-4-yl]-3,6-dihydro-2H-1,3,4-oxadiazin-2-one,
5-[3-(difluoromethyl)-4-(6-methylpyridin-3-yl)phenyl]-3,6-dihydro-2H-1,3,4-oxadiazin-2-one,
5-[4-(cyclopent-1-en-1-yl)-3-(difluoromethyl)phenyl]-3,6-dihydro-2H-1,3,4-oxadiazin-2-one,
5-[3-(difluoromethyl)-4-(1H-pyrazol-4-yl)phenyl]-3,6-dihydro-2H-1,3,4-oxadiazin-2-one,
5-[4-(3-Hydroxy-3-methylazetidin-1-yl)-3-(trifluoromethyl)phenyl]-3,6-dihydro-2H-1,3,4-oxadiazin-2-one,
(rac)-5-[4-{[3,3,3-trifluoro-2-hydroxypropyl]amino}-3-(trifluoromethyl)phenyl]-3,6-dihydro-2H-1,3,4-oxadiazin-2-one,
5-{4-[(Oxan-4-yl)amino]-3-(trifluoromethyl)phenyl}-3,6-dihydro-2H-1,3,4-oxadiazin-2-one,
5-[4-{[(cis/trans)-3-hydroxycyclobutyl]amino}-3-(trifluoromethyl)phenyl]-3,6-dihydro-2H-1,3,4-oxadiazin-2-one,
5-{4-[(rac)-2,4-Dimethylazetidin-1-yl]-3-(trifluoromethyl)phenyl}-3,6-dihydro-2H-1,3,4-oxadiazin-2-one,
5-{4-[(cis or trans)-2,4-Dimethylazetidin-1-yl]-3-(trifluoromethyl)phenyl}-3,6-dihydro-2H-1,3,4-oxadiazin-2-one,
5-[4-{[3,3,3-Trifluoro-2(S)-hydroxypropyl]amino}-3-(trifluoromethyl)phenyl]-3,6-dihydro-2H-1,3,4-oxadiazin-2-one,
5-{4-[(2-Hydroxy-2-methylpropyl)amino]-3-(trifluoromethyl)phenyl}-3,6-dihydro-2H-1,3,4-oxadiazin-2-one,
5-[4-{[(trans)-4-Hydroxycyclohexyl]amino}-3-(trifluoromethyl)phenyl]-3,6-dihydro-2H-1,3,4-oxadiazin-2-one,
5-{4-[(Cyclopropylmethyl)amino]-3-(trifluoromethyl)phenyl}-3,6-dihydro-2H-1,3,4-oxadiazin-2-one,
5-[4-{[(3-Methyloxetan-3-yl)methyl]amino}-3-(trifluoromethyl)phenyl]-3,6-dihydro-2H-1,3,4-oxadiazin-2-one,
-{4-[(3-Methoxypropyl)amino]-3-(trifluoromethyl)phenyl}-3,6-dihydro-2H-1,3,4-oxadiazin-2-one,
5-[4-({[(rac)-Oxolan-2-yl]methyl}amino)-3-(trifluoromethyl)phenyl]-3,6-dihydro-2H-1,3,4-oxadiazin-2-one,
5-[4-{[2(R)-2-Hydroxypropyl]amino}-3-(trifluoromethyl)phenyl]-3,6-dihydro-2H-1,3,4-oxadiazin-2-one,
5-[4-{[(3R)-3-Hydroxybutyl]amino}-3-(trifluoromethyl)phenyl]-3,6-dihydro-2H-1,3,4-oxadiazin-2-one,
5-[4-{[(2S)-2-Hydroxypropyl]amino}-3-(trifluoromethyl)phenyl]-3,6-dihydro-2H-1,3,4-oxadiazin-2-one,
5-[4-{[(1-Hydroxycyclobutyl)methyl]amino}-3-(trifluoromethyl)phenyl]-3,6-dihydro-2H-1,3,4-oxadiazin-2-one,
5-{4-[(3-Methylbutyl)amino]-3-(trifluoromethyl)phenyl}-3,6-dihydro-2H-1,3,4-oxadiazin-2-one,
5-{4-[(2-Methylpropyl)amino]-3-(trifluoromethyl)phenyl}-3,6-dihydro-2H-1,3,4-oxadiazin-2-one,
5-{4-[(2-Methoxyethyl)amino]-3-(trifluoromethyl)phenyl}-3,6-dihydro-2H-1,3,4-oxadiazin-2-one,
5-{4-[Ethyl(methyl)amino]-3-(trifluoromethyl)phenyl}-3,6-dihydro-2H-1,3,4-oxadiazin-2-one,
5-[4-(tert-butylamino)-3-(trifluoromethyl)phenyl]-3,6-dihydro-2H-1,3,4-oxadiazin-2-one,
5-[4-({[(2R)-oxolan-2-yl]methyl}amino)-3-(trifluoromethyl)phenyl]-3,6-dihydro-2H-1,3,4-oxadiazin-2-one,
5-[4-{[(pyrazin-2-yl)methyl]amino}-3-(trifluoromethyl)phenyl]-3,6-dihydro-2H-1,3,4-oxadiazin-2-one,
5-[4-(4-hydroxypiperidin-1-yl)-3-(trifluoromethyl)phenyl]-3,6-dihydro-2H-1,3,4-oxadiazin-2-one,
5-[4-{[(2S)-1-hydroxybutan-2-yl]amino}-3-(trifluoromethyl)phenyl]-3,6-dihydro-2H-1,3,4-oxadiazin-2-one,
5-[4-(3-hydroxypiperidin-1-yl)-3-(trifluoromethyl)phenyl]-3,6-dihydro-2H-1,3,4-oxadiazin-2-one (racemic mixture),
(rac)-1-[4-(2-oxo-3,6-dihydro-2H-1,3,4-oxadiazin-5-yl)-2-(trifluoromethyl)phenyl]piperidine-3-carboxamide,
5-{4-[(3-hydroxy-2,2-dimethylpropyl)amino]-3-(trifluoromethyl)phenyl}-3,6-dihydro-2H-1,3,4-oxadiazin-2-one,
5-[4-(4,4-difluoropiperidin-1-yl)-3-(trifluoromethyl)phenyl]-3,6-dihydro-2H-1,3,4-oxadiazin-2-one,
5-[4-{[(1R,2R,4R)-bicyclo[2.2.1]heptan-2-yl]amino}-3-(trifluoromethyl)phenyl]-3,6-dihydro-2H-1,3,4-oxadiazin-2-one,
5-{4-[(3S)-3-hydroxypyrrolidin-1-yl]-3-(trifluoromethyl)phenyl}-3,6-dihydro-2H-1,3,4-oxadiazin-2-one,
(rac)-5-{4-[(2-hydroxy-3-methoxypropyl)amino]-3-(trifluoromethyl)phenyl}-3,6-dihydro-2H-1,3,4-oxadiazin-2-one,
5-[4-{[(1-methyl-1H-pyrazol-5-yl)methyl]amino}-3-(trifluoromethyl)phenyl]-3,6-dihydro-2H-1,3,4-oxadiazin-2-one,
5-[4-{[(1H-pyrazol-3-yl)methyl]amino}-3-(trifluoromethyl)phenyl]-3,6-dihydro-2H-1,3,4-oxadiazin-2-one,
5-[4-{[2-(1H-pyrazol-1-yl)ethyl]amino}-3-(trifluoromethyl)phenyl]-3,6-dihydro-2H-1,3,4-oxadiazin-2-one,
1-[4-(2-oxo-3,6-dihydro-2H-1,3,4-oxadiazin-5-yl)-2-(trifluoromethyl)phenyl]piperidine-4-carbonitrile,
(rac)-5-{4-[(1-cyclopropylethyl)amino]-3-(trifluoromethyl)phenyl}-3,6-dihydro-2H-1,3,4-oxadiazin-2-one,
(rac)-5-{4-[(2-ethoxypropyl)amino]-3-(trifluoromethyl)phenyl}-3,6-dihydro-2H-1,3,4-oxadiazin-2-one
(rac)-5-{4-[(2-methoxypropyl)amino]-3-(trifluoromethyl)phenyl}-3,6-dihydro-2H-1,3,4-oxadiazin-2-one
5-[4-(3-ethoxyazetidin-1-yl)-3-(trifluoromethyl)phenyl]-3,6-dihydro-2H-1,3,4-oxadiazin-2-one,
5-[4-{[(pyrimidin-2-yl)methyl]amino}-3-(trifluoromethyl)phenyl]-3,6-dihydro-2H-1,3,4-oxadiazin-2-one,
5-[4-{[(oxolan-3-yl)methyl]amino}-3-(trifluoromethyl)phenyl]-3,6-dihydro-2H-1,3,4-oxadiazin-2-one (racemic mixture),
5-[4-{[(2S)-4-hydroxybutan-2-yl]amino}-3-(trifluoromethyl)phenyl]-3,6-dihydro-2H-1,3,4-oxadiazin-2-one,
(rac)-5-[4-{[(6-oxopiperidin-3-yl)methyl]amino}-3-(trifluoromethyl)phenyl]-3,6-dihydro-2H-1,3,4-oxadiazin-2-one,
(rac)-5-[4-{[(2,2-dimethylcyclopropyl)methyl]amino}-3-(trifluoromethyl)phenyl]-3,6-dihydro-2H-1,3,4-oxadiazin-2-one,
5-[4-({[1-(hydroxymethyl)cyclobutyl]methyl}amino)-3-(trifluoromethyl)phenyl]-3,6-dihydro-2H-1,3,4-oxadiazin-2-one,
5-{4-[(2S)-2-(hydroxymethyl)azetidin-1-yl]-3-(trifluoromethyl)phenyl}-3,6-dihydro-2H-1,3,4-oxadiazin-2-one,
3-methyl-1-[4-(2-oxo-3,6-dihydro-2H-1,3,4-oxadiazin-5-yl)-2-(trifluoromethyl)phenyl]azetidine-3-carbonitrile,
5-[4-(3-azabicyclo[3.1.0]hexan-3-yl)-3-(trifluoromethyl)phenyl]-3,6-dihydro-2H-1,3,4-oxadiazin-2-one,
5-[4-(4-ethyl-4-hydroxypiperidin-1-yl)-3-(trifluoromethyl)phenyl]-3,6-dihydro-2H-1,3,4-oxadiazin-2-one,
4-[4-(2-oxo-3,6-dihydro-2H-1,3,4-oxadiazin-5-yl)-2-(trifluoromethyl)anilino]butanenitrile,
6-[4-(2-oxo-3,6-dihydro-2H-1,3,4-oxadiazin-5-yl)-2-(trifluoro-methyl)phenyl]-2lambda⁶-thia-6-azaspiro[3.3]heptane-2,2-dione,
N²-[4-(2-oxo-3,6-dihydro-2H-1,3,4-oxadiazin-5-yl)-2-(trifluoromethyl)phenyl]glycinamide,
5-{4-[(3R)-3-hydroxypyrrolidin-1-yl]-3-(trifluoromethyl)phenyl}-3,6-dihydro-2H-1,3,4-oxadiazin-2-one,
5-{4-[(2-methoxy-2-methylpropyl)amino]-3-(trifluoromethyl)phenyl}-3,6-dihydro-2H-1,3,4-oxadiazin-2-one
5-[4-({[(2S)-oxolan-2-yl]methyl}amino)-3-(trifluoromethyl)phenyl]-3,6-dihydro-2H-1,3,4-oxadiazin-2-one,
5-{4-[(2-ethoxyethyl)amino]-3-(trifluoromethyl)phenyl}-3,6-dihydro-2H-1,3,4-oxadiazin-2-one,
5-[4-{[(1S,2R)-2-hydroxycyclopentyl]amino}-3-(trifluoromethyl)phenyl]-3,6-dihydro-2H-1,3,4-oxadiazin-2-one,
5-{4-[(oxetan-3-yl)amino]-3-(trifluoromethyl)phenyl}-3,6-dihydro-2H-1,3,4-oxadiazin-2-on,
5-{3-(difluoromethyl)-4-[1-(propan-2-yl)-1H-pyrazol-4-yl]phenyl}-3,6-dihydro-2H-1,3,4-oxadiazin-2-one,
5-[3-fluoro-4-(morpholin-4-yl)-5-(trifluoromethyl)phenyl]-3,6-dihydro-2H-1,3,4-oxadiazin-2-one,
(6S)-6-methyl-5-{3-(trifluoromethyl)-4-[3-(trifluoromethyl)-1H-pyrazol-1-yl]phenyl}-3,6-dihydro-2H-1,3,4-oxadiazin-2-one,
(6S)-6-methyl-5-{3-(trifluoromethyl)-4-[4-(trifluoromethyl)-1H-imidazol-1-yl]phenyl}-3,6-dihydro-2H-1,3,4-oxadiazin-2-one,
5-[4-(3-Methoxypropyl)-3-(trifluoromethyl)phenyl]-3,6-dihydro-2H-1,3,4-oxadiazin-2-one,
5-[4-(2-methylpropyl)-3-(trifluoromethyl)phenyl]-3,6-dihydro-2H-1,3,4-oxadiazin-2-one,
5-[4-(3,3-dimethylbutyl)-3-(trifluoromethyl)phenyl]-3,6-dihydro-2H-1,3,4-oxadiazin-2-one,
5-[4-(Propan-2-yl)-3-(trifluoromethyl)phenyl]-3,6-dihydro-2H-1,3,4-oxadiazin-2-one,
(rac)-5-{4-[oxan-3-yl]-3-(trifluoromethyl)phenyl}-3,6-dihydro-2H-1,3,4-oxadiazin-2-one,
(trans)-5-{4-[4-hydroxycyclohexyl]-3-(trifluoromethyl)phenyl}-3,6-dihydro-2H-1,3,4-oxadiazin-2-one (trans isomer),
(cis)-5-{4-[4-hydroxycyclohexyl]-3-(trifluoromethyl)phenyl}-3,6-dihydro-2H-1,3,4-oxadiazin-2-one,
5-{4-[(2-Aminoethyl)amino]-3-(trifluoromethyl)phenyl}-3,6-dihydro-2H-1,3,4-oxadiazin-2-one-salt with hydrochloric acid,
5-{4-[1-amino-3-azabicyclo[3.1.0]hexan-3-yl]-3-(trifluoromethyl)-phenyl}-3,6-dihydro-2H-1,3,4-oxadiazin-2-one-salt with hydrochloric acid,
5-[4-(methylamino)-3-(trifluoromethyl)phenyl]-3,6-dihydro-2H-1,3,4-oxadiazin-2-one,
(6S)-6-methyl-5-[4-(4-methylpiperazin-1-yl)-3-(trifluoromethyl)phenyl]-3,6-dihydro-2H-1,3,4-oxadiazin-2-one,
5-[4-(2-hydroxypropan-2-yl)-3-(trifluoromethyl)phenyl]-3,6-dihydro-2H-1,3,4-oxadiazin-2-one,
(6S)-5-[4-(3,3-difluoroazetidin-1-yl)-3-(trifluoromethyl)phenyl]-6-methyl-3,6-dihydro-2H-1,3,4-oxadiazin-2-one and
(6S)-5-[4-(3-hydroxy-3-methylazetidin-1-yl)-3-(trifluoromethyl)-phenyl]-6-methyl-3,6-dihydro-2H-1,3,4-oxadiazin-2-one
or a stereoisomer, a tautomer, a hydrate, a solvate, or a salt thereof, or a mixture of same.

11. The method of treatment according to claim 9, wherein the compound is selected from the group
5-[4-(4,4-Difluoropiperidin-1-yl)-3-fluorophenyl]-3,6-dihydro-2H-1,3,4-oxadiazin-2-one,
5-[4′-fluoro-2-(trifluoromethyl)biphenyl-4-yl]-3,6-dihydro-2H-1,3,4-oxadiazin-2-one,
5-[3-Fluoro-4-(morpholin-4-yl)phenyl]-3,6-dihydro-2H-1,3,4-oxadiazin-2-one,
5-(4′-fluoro-2-methylbiphenyl-4-yl)-3,6-dihydro-2H-1,3,4-oxadiazin-2-one,
5-(3′,4′-Difluoro-2-methylbiphenyl-4-yl)-3,6-dihydro-2H-1,3,4-oxadiazin-2-one,
5-[4′-(Difluoromethyl)-2-fluoro[1,1′-biphenyl]-4-yl]-3,6-dihydro-2H-1,3,4-oxadiazin-2-one,
(6S)-5-[4′-fluoro-2-(trifluoromethyl)biphenyl-4-yl]-6-methyl-3,6-dihydro-2H-1,3,4-oxadiazin-2-one,
5-{4-[1-(difluoromethyl)-1H-pyrazol-4-yl]-3-(trifluoromethyl)phenyl}-3,6-dihydro-2H-1,3,4-oxadiazin-2-one,
5-[4-Methyl-3-(trifluoromethyl)phenyl]-3,6-dihydro-2H-1,3,4-oxadiazin-2-one
(6S)-6-methyl-5-{3-(trifluoromethyl)-4-[3-(trifluoromethyl)-1H-pyrazol-1-yl]phenyl}-3,6-dihydro-2H-1,3,4-oxadiazin-2-one,
5-[4-(3-Methoxypropyl)-3-(trifluoromethyl)phenyl]-3,6-dihydro-2H-1,3,4-oxadiazin-2-one,
-[4-(3,3-dimethylbutyl)-3-(trifluoromethyl)phenyl]-3,6-dihydro-2H-1,3,4-oxadiazin-2-one,
aid
(6S)-6-methyl-5-[4-(4-methylpiperazin-1-yl)-3-(trifluoromethyl)phenyl]-3,6-dihydro-2H-1,3,4-oxadiazin-2-one,
or a stereoisomer, a tautomer, a hydrate, a solvate, or a salt thereof, or a mixture of same.

12. The method of claim 9, wherein the compound is selected from the group
(6S)-5-[4′-fluoro-2-(trifluoromethyl)biphenyl-4-yl]-6-methyl-3,6-dihydro-2H-1,3,4-oxadiazin-2-one,
5-{4-[1-(difluoromethyl)-1H-pyrazol-4-yl]-3-(trifluoromethyl)phenyl}-3,6-dihydro-2H-1,3,4-oxadiazin-2-one,
(6S)-6-methyl-5-{3-(trifluoromethyl)-4-[3-(trifluoromethyl)-1H-pyrazol-1-yl]phenyl}-3,6-dihydro-2H-1,3,4-oxadiazin-2-one,
or a stereoisomer, a tautomer, a hydrate, a solvate, or a salt thereof, or a mixture of same.

13. The method of claim 9, wherein the compound is
(6S)-5-[4′-fluoro-2-(trifluoromethyl)biphenyl-4-yl]-6-methyl-3,6-dihydro-2H-1,3,4-oxadiazin-2-one,
or a stereoisomer, a tautomer, a hydrate, a solvate, or a salt thereof, or a mixture of same.

14. The method according to claim 1, whereby the compound mentioned therein is comprised in a pharmaceutical composition together with one or more pharmaceutically acceptable excipients.

15. The method according to claim 1, whereby the compound is combined in a pharmaceutical combination comprising:
one or more first active ingredients of general formula (I), and
one or more further active ingredients.

16. A method for controlling sarcoma in a subject by administering an effective amount of at least one compound as defined in claim 1.

17. The method according to claim 1, whereby the sarcoma is sarcoma of a soft tissue or bone.

18. The method according to claim 1, whereby the sarcoma is selected from malignant fibrous histiocytoma, osteosarcoma, sarcoma, soft tissue sarcoma and synovial sarcoma.

19. A method of inhibiting the growth or proliferation of a sarcoma in a subject, the method comprising administering to a subject having a sarcoma selected from the group consisting of osteosarcoma, synovial sarcoma, soft tissue sarcoma, a sarcoma represented by a recurrent malignant solitary fibrous tumor and fibrous histiocytoma an effective amount of a pharmaceutical composition comprising (6S)-5-[4′-fluoro-2-(trifluoromethyl)biphenyl-4-yl]-6-methyl-3,6-dihydro-2H-1,3,4-oxadiazin-2-one, or a stereoisomer, a tautomer, a hydrate, a solvate, or a salt thereof, or a mixture of same.

20. The method according to claim 16, wherein the compound is
(6S)-5-[4′-fluoro-2-(trifluoromethyl)biphenyl-4-yl]-6-methyl-3,6-dihydro-2H-1,3,4-oxadiazin-2-one,
or a stereoisomer, a tautomer, a hydrate, a solvate, or a salt thereof, or a mixture of same.
